SEQUENCE LISTING (10 sequences)

SEQ ID NO: 1 - DNA, 22 bp, Artificial Sequence
  Description of Artificial Sequence Synthetic primer
gagaaatccaaagagcaaatgg

SEQ ID NO: 2 - DNA, 21 bp, Artificial Sequence
  Description of Artificial Sequence Synthetic primer
agaatggaaaactggaatagg

SEQ ID NO: 3 - DNA, 20 bp, Artificial Sequence
  Description of Artificial Sequence Synthetic primer
tcttcgatgcttcggattcc

SEQ ID NO: 4 - DNA, 21 bp, Artificial Sequence
  Description of Artificial Sequence Synthetic primer
gaattctcggaatctctgttg

SEQ ID NO: 5 - DNA, 21 bp, Artificial Sequence
  Description of Artificial Sequence Synthetic primer
ttacaagcagtgcagaaaacc

SEQ ID NO: 6 - DNA, 22 bp, Artificial Sequence
  Description of Artificial Sequence Synthetic primer
agtaaacattgaaaccacagcc

SEQ ID NO: 7 - DNA, 20 bp, Artificial Sequence
  Description of Artificial Sequence Synthetic primer
tctgcagctctgtgtgaagg

SEQ ID NO: 8 - DNA, 22 bp, Artificial Sequence
  Description of Artificial Sequence Synthetic primer
cttcaaaaacttctccacaacc

SEQ ID NO: 9 - DNA, 17 bp, Artificial Sequence
  Description of Artificial Sequence Synthetic primer
cccacgccacgctctcc

SEQ ID NO: 10 - DNA, 19 bp, Artificial Sequence
  Description of Artificial Sequence Synthetic primer
tcctgtcagttggtgctcc

(19) United States
(12) Patent Application Publication
(10) Pub. No.: US 2016/0158293 A1
(43) Pub. Date: Jun. 9, 2016

(54) Treatment of Ocular Conditions Using Progenitor Cells

(71) Applicant: JANSSEN BIOTECH, INC., HORSHAM, PA (US)

(72) Inventors: IAN R. HARRIS, SPRING HOUSE, PA (US); NADINE DEJNEKA, SPRING HOUSE, PA (US)

(73) Assignee: JANSSEN BIOTECH, INC., HORSHAM, PA (US)

(21) Appl. No.: 14/960,055

(22) Filed: Dec. 4, 2015

Related U.S. Application Data
(60) Provisional application No. 62/088,429, filed on Dec. 5, 2014.
(60) Provisional application No. 62/126,370, filed on Feb. 27, 2015.
(60) Provisional application No. 62/220,873, filed on Sep. 18, 2015.

(51) Int. Cl. A61K 35/51; A61K 9/00

(52) U.S. Cl. CPC A61K 35/51; A61K 9/0048

(57) ABSTRACT

Methods and compositions for treating ophthalmic disease, promoting development of functional neuronal synapses and improving neuronal outgrowth using progenitor cells, such as postpartum-derived cells, and conditioned media from the cells, are disclosed. Trophic factors and other agents secreted by the cells that promote synapse formation and neuronal growth are also disclosed.

### CROSS REFERENCE TO RELATED APPLICATION

This application claims benefit of U.S. Provisional Application Ser. No. 62/088,429, filed Dec. 5, 2014, U.S. Provisional Application Ser. No. 62/126,370, filed Feb. 27, 2015, and U.S. Provisional Application Ser. No. 62/220,873, filed Sep. 18, 2015, the entire contents of each is incorporated by reference herein in its entirety.

### FIELD OF THE INVENTION

This invention relates to the field of cell-based or regenerative therapy for ophthalmic diseases and disorders, particularly ocular conditions, such as retinal degenerative conditions. The invention provides methods and compositions for the regeneration or repair of ocular cells and tissue using progenitor cells, such as umbilical cord tissue-derived cells, placenta tissue-derived cells, and conditioned media prepared from those cells.

### BACKGROUND

As a complex and sensitive organ of the body, the eye can experience numerous diseases and other deleterious conditions that affect its ability to function normally. Many of these conditions are associated with damage or degeneration of specific ocular cells, and tissues made up of those cells. As one example, diseases and degenerative conditions of the optic nerve and retina are the leading causes of blindness throughout the world. Damage or degeneration of the cornea, lens and associated ocular tissues represent another significant cause of vision loss worldwide.

The retina contains seven layers of alternating cells and processes that convert a light signal into a neural signal. The retinal photoreceptors and adjacent retinal pigment epithelium (RPE) form a functional unit that, in many disorders, becomes unbalanced due to genetic mutations or environmental conditions (including age). This results in loss of photoreceptors through apoptosis or secondary degeneration, which leads to progressive deterioration of vision and, in some instances, to blindness (for a review, see, e.g., Lund, R. D. et al., Progress in Retinal and Eye Research, 2001; 20:415-449). Two classes of ocular disorders that fall into this pattern are age-related macular degeneration (AMD) and retinitis pigmentosa (RP).

AMD is the most common cause of vision loss in the United States in those people whose ages are 50 or older, and its prevalence increases with age. The primary disorder in AMD appears to be due to RPE dysfunction and changes in Bruch's membranes, characterized by, among other things, lipid deposition, protein cross-linking and decreased permeability to nutrients (see Lund et al., 2001 supra). A variety of elements may contribute to macular degeneration, including genetic makeup, age, nutrition, smoking and exposure to sunlight. The nonexudative, or “dry” form of AMD accounts for 90% of AMD cases; the other 10% being the exudative-neovascular form (“wet” AMD). In dry-AMD patients, there is a gradual disappearance of the retinal pigment epithelium (RPE), resulting in circumscribed areas of atrophy. Since photoreceptor loss follows the disappearance of RPE, the affected retinal areas have little or no visual function.

Current therapies for AMD involve procedures, such as, for example, laser therapy and pharmacological intervention. By transferring thermal energy, the laser beam destroys the leaky blood vessels under the macula, slowing the rate of vision loss. A disadvantage of laser therapy is that the high thermal energy delivered by the beam also destroys healthy tissue nearby. Neuroscience 4^{th} edition, (Purves, D, et al. 2008) states “[c]urrently there is no treatment for dry AMD.”

RPE transplantation has been unsuccessful in humans. For example, Zarbin, M, 2003 states, “[w]ith normal aging, human Bruch's membrane, especially in the submacular region, undergoes numerous changes (e.g., increased thickness, deposition of ECM and lipids, cross-linking of protein, non-enzymatic formation of advanced glycation end products). These changes and additional changes due to AMD could decrease the bioavailability of ECM ligands (e. g., laminin, fibronectin, and collagen IV) and cause the extremely poor survival of RPE cells in eyes with AMD. Thus, although human RPE cells express the integrins needed to attach to these ECM molecules, RPE cell survival on aged submacular human Bruch's membrane is impaired.” (Zarbin, M A, Trans Am Ophthalmol Soc, 2003; 101:493-514).

Retinitis pigmentosa is mainly considered an inherited disease, with over 100 mutations being associated with photoreceptor loss (see Lund et al., 2001, supra). Though the majority of mutations target photoreceptors, some affect RPE cells directly. Together, these mutations affect such processes as molecular trafficking between photoreceptors and RPE cells and phototransduction.

Other less common, but nonetheless debilitating retinopathies can also involve progressive cellular degeneration leading to vision loss and blindness. These include, for example, diabetic retinopathy and choroidal neovascular membrane (CNVM).

The advent of stem cell-based therapy for cell and tissue repair and regeneration provides promising treatments for a number of aforementioned cell-degenerative pathologies and other retinal conditions. Stem cells are capable of self-renewal and differentiation to generate a variety of mature cell lineages. Transplantation of such cells can be utilized as a clinical tool for reconstituting a target tissue, thereby restoring physiologic and anatomic functionality. The application of stem cell technology is wide-ranging, including tissue engineering, gene therapy delivery, and cell therapeutics, i.e., delivery of biotherapeutic agents to a target location via exogenously supplied living cells or cellular components that produce or contain those agents. (For a review, see, for example, Tresco, P. A. et al., Advanced Drug Delivery Reviews, 2000, 42: 2-37).

Cell therapy demonstrates great potential for the treatment of neurological disorders. Transplanted cells are thought to promote recovery and neuroprotection by replacing the damaged cells or by providing trophic factors that enhance neural health and regeneration. (Doeppner T R, Hermann D M, Frontiers in Cellular Neuroscience, 2014; 8:357; Atala A, Lancet, 2014/2015; 385(9967):487-488; Popovich P G, Cell, 2012; 150:1105-1106; Lindvall O, et al., Journal of Clinical Investigation, 2010; 120:29-40; Rao M S, Mechanisms of Aging Development, 2001; 122:713-734). Atrophy of neuronal processes is a universal event in many neurological disorders; therefore, providing factors that can trigger neurite outgrowth can provide therapeutic effects in these diseases. Specifically, human umbilical tissue-derived cells (hUTC) may be a promising treatment for neuronal loss. Human umbilical cord may be harvested from postpartum umbilical cords without ethical concerns, are shown to have karyotypic stability during in vitro culture, and can be expanded (Lund et al., Stem Cells, 2007; 25(3):602-61). The therapeutic potential of hUTC administration has been demonstrated in various animal disease models. For example, delivery of hUTC into animal models of stroke (Moore et al., Somatosensory and Motor Research, 2013; 30:185-196; Zhang L, et al., Brain Research, 2012; 1489:104-112; Zhang L, et al., Cell transplantation, 2013; 22:1569-1576; Jiang Q, et al., PloS One, 2012; 7(8):e42845; Zhang L, et al., Stroke; 2011; 42:1437-1444); and retinal degeneration (Lund et al., Stem Cells, 2007 supra) have shown that these cells enhance functional recovery and protect neurons from progressive degeneration and cell death.

Recently, it has been shown that postpartum-derived cells ameliorate retinal degeneration (US 2010/0272803). The Royal College of Surgeons (RCS) rat presents with a tyrosine receptor kinase (Mertk) defect affecting outer segment phagocytosis, leading to photoreceptor cell death. (Feng W. et al., J Biol Chem., 2002, 10: 277 (19): 17016-17022). Transplantation of retinal pigment epithelial (RPE) cells into the subretinal space of RCS rats was found to limit the progress of photoreceptor loss and preserve visual function. It also has been demonstrated that postpartum-derived cells can be used to promote photoreceptor rescue and thus preserve photoreceptors in the RCS model. (US 2010/0272803). Injection of human umbilical cord tissue-derived cells (hUTCs) subretinally into RCS rat eye improved visual acuity and ameliorated retinal degeneration. Moreover, treatment with conditioned medium (CM) derived from hUTC restored phagocytosis of ROS in dystrophic RPE cells in vitro. (US 2010/0272803). The mechanism for hUTC improving vision is further investigated here.

### SUMMARY

This invention provides compositions and methods applicable to cell-based or regenerative therapy for ophthalmic diseases and disorders. In particular, the invention features methods and compositions, including pharmaceutical compositions, for treating an ophthalmic disease or condition, including the regeneration or repair of ocular cells and tissue, using progenitor cells such as postpartum-derived cells, and conditioned media generated from those cells. The postpartum-derived cells may be umbilical cord tissue-derived cells or placental tissue-derived cells.

One aspect of the invention is a method of treating ophthalmic disease by inducing synaptogenesis in neuronal cells comprising administering a population of progenitor cells or a conditioned medium prepared from a population of progenitor cells. In an embodiment of the invention, the neuronal cells or neurons are retinal neurons such as retinal ganglion cells, photoreceptors (rods and cones), retina amicrine cells, horizontal cells or bipolar cells. In particular embodiments of the invention, the progenitor cells are postpartum-derived cells. In embodiments of the invention, the postpartum-derived cells are isolated from human umbilical cord tissue or placental tissue substantially free of blood. In a further embodiment, the progenitor cells secrete trophic factors. In an embodiment, the conditioned media contains trophic factors secreted by the progenitor cell population. In embodiments, trophic factors secreted by the progenitor cells, such as postpartum-derived cells, induce synaptogenesis. In embodiments, the trophic factors are selected from thrombospondin-1, thrombospondin-2, and thrombospondin-4.

In an aspect of the invention, the method of treating ophthalmic disease by inducing neurite outgrowth in neuronal cells comprising administering a population of progenitor cells or a conditioned medium prepared from a population of progenitor cells. In an embodiment of the invention, the neuronal cells or neurons are retinal neurons, such as retinal ganglion cells, photoreceptors (rods and cones), retina amicrine cells, horizontal cells or bipolar cells. In particular embodiments of the invention, the progenitor cells are postpartum-derived cells. In embodiments of the invention, the postpartum-derived cells are isolated from human umbilical cord tissue or placental tissue substantially free of blood. In a further embodiment, the progenitor cells secrete trophic factors. In an embodiment, the conditioned media contains trophic factors secreted by the progenitor cell population. In embodiments, trophic factors secreted by the progenitor cells, such as postpartum-derived cells, induce synaptogenesis. In embodiments, the trophic factors are selected from thrombospondin-1, thrombospondin-2, and thrombospondin-4.

A further aspect of the invention is a method of inducing neurite outgrowth comprising administering a population of progenitor cells or a conditioned media prepared from a population of progenitor cells. In an embodiment of the invention, the neuronal cells (neurons) are retinal neurons, for example, retinal ganglion cells, photoreceptors (rods and cones), retina amicrine cells, horizontal cells or bipolar cells. In embodiments of the invention, the progenitor cells are postpartum-derived cells. In embodiments, the postpartum-derived cells are isolated from human umbilical cord tissue or placental tissue substantially free of blood. In a further embodiment, the progenitor cells secrete trophic factors. In an embodiment, the conditioned media contains trophic factors secreted by the progenitor cell population. In embodiments, trophic factors secreted by the progenitor cells, such as postpartum-derived cells, induces neurite outgrowth. In a further embodiment, the trophic factors secreted by the progenitor cells are selected from thrombospondin-1, thrombospondin-2, and thrombospondin-4.

Another embodiment of the invention is a method of developing functional synapses in retinal neurons comprising administering a population of progenitor cells or a conditioned media prepared from a population of progenitor cells. In embodiments of the invention, the retinal neurons are retinal ganglion cells, photoreceptors (rods and cones), retina amicrine cells, horizontal cells or bipolar cells, and the progenitor cells are postpartum-derived cells. In embodiments, the postpartum-derived cells are isolated from human umbilical cord tissue or placental tissue substantially free of blood. In a further embodiment, the progenitor cells secrete trophic factors. In an embodiment, the conditioned media contains trophic factors secreted by the progenitor cell population. In embodiments, trophic factors secreted by the progenitor cells, such as postpartum-derived cells, induce neurite outgrowth. In a further embodiment, the trophic factors secreted by the progenitor cells are selected from thrombospondin-1, thrombospondin-2, and thrombospondin-4.

In embodiments of the invention herein, conditioned media prepared from a population of progenitor cells, for example postpartum-derived cells, contains trophic factors secreted by the cell population. Such trophic factors secreted by the cells are selected from thrombospondin-1, thrombospondin-2, and thrombospondin-4. The postpartum-derived cells are umbilical cord tissue-derived cells (UTCs) or placental tissue-derived cells (PDCs).

In one embodiment, progenitor cells promote the development of functional synapses in retinal neurons. In another embodiment, trophic factors secreted by progenitor cells promote the development of functional synapses in neurons. In a specific embodiment, the trophic factors are selected from thrombospondin-1, thrombospondin-2, and thrombospondin-4. In yet another embodiment, a population of progenitor cells supports growth of neuronal cells.

In a further embodiment, conditioned media prepared from the population of progenitor cells supports growth of neuronal cells. In the embodiments of the invention, conditioned media prepared from the population of progenitor cells described above contains trophic factors secreted by the cell population. In a specific embodiment, the trophic factors are selected from thrombospondin-1, thrombospondin-2, and thrombospondin-4.

Another aspect of the invention features a method for promoting development of synapses in retinal degeneration, the method comprising administering to a subject a conditioned media in an amount effective to promote development of synapses. In an embodiment of the invention, the conditioned media is prepared from a population of postpartum-derived cells. In a particular embodiment, the postpartum-derived cells are isolated from human umbilical cord tissue or placental tissue substantially free of blood. As in other embodiments, the conditioned media contains trophic factors secreted by the cell population. Such trophic factors secreted by the cells are selected from thrombospondin-1, thrombospondin-2, and thrombospondin-4.

In the embodiments of the invention, the postpartum-derived cell is derived from human umbilical cord tissue or placental tissue substantially free of blood. In embodiments, the cell is capable of expansion in culture and has the potential to differentiate into a cell of a neural phenotype; wherein the cell requires L-valine for growth and is capable of growth in at least about 5% oxygen. The cell further comprises one or more of the following characteristics: (a) potential for at least about 40 doublings in culture; (b) attachment and expansion on a coated or uncoated tissue culture vessel, wherein the coated tissue culture vessel comprises a coating of gelatin, laminin, collagen, polyomithine, vitronectin, or fibronectin; (c) production of at least one of tissue factor, vimentin, and alpha-smooth muscle actin; (d) production of at least one of CD10, CD13, CD44, CD73, CD90, PDGFr-alpha, PD-L2 and HLA-A,B,C; (e) lack of production of at least one of CD31, CD34, CD45, CD80, CD86, CD117, CD141, CD178, B7-H2, HLA-G, and HLA-DR,DP,DQ, as detected by flow cytometry; (f) expression of a gene, which relative to a human cell that is a fibroblast, a mesenchymal stem cell, or an iliac crest bone marrow cell, is increased for at least one of a gene encoding: interleukin 8; reticulon 1; chemokine (C--X--C motif) ligand 1 (melonoma growth stimulating activity, alpha); chemokine (C--X--C motif) ligand 6 (granulocyte chemotactic protein 2); chemokine (C--X--C motif) ligand 3; tumor necrosis factor, alpha-induced protein 3; C-type lectin superfamily member 2; Wilms tumor 1; aldehyde dehydrogenase 1 family member A2; renin; oxidized low density lipoprotein receptor 1; Homo sapiens clone IMAGE:4179671; protein kinase C zeta; hypothetical protein DKFZp564F013; downregulated in ovarian cancer 1; and Homo sapiens gene from clone DKFZp547k1113; (g) expression of a gene, which relative to a human cell that is a fibroblast, a mesenchymal stem cell, or an iliac crest bone marrow cell, is reduced for at least one of a gene encoding: short stature homeobox 2; heat shock 27 kDa protein 2; chemokine (C--X--C motif) ligand 12 (stromal cell-derived factor 1); elastin (supravalvular aortic stenosis, Williams-Beuren syndrome); Homo sapiens mRNA; cDNA DKFZp586M2022 (from clone DKFZp586M2022); mesenchyme homeo box 2 (growth arrest-specific homeo box); sine oculis homeobox homolog 1 (Drosophila); crystallin, alpha B; disheveled associated activator of morphogenesis 2; DKFZP586B2420 protein; similar to neuralin 1; tetranectin (plasminogen binding protein); src homology three (SH3) and cysteine rich domain; cholesterol 25-hydroxylase; runt-related transcription factor 3; interleukin 11 receptor, alpha; procollagen C-endopeptidase enhancer; frizzled homolog 7 (Drosophila); hypothetical gene BC008967; collagen, type VIII, alpha 1; tenascin C (hexabrachion); iroquois homeobox protein 5; hephaestin; integrin, beta 8; synaptic vesicle glycoprotein 2; neuroblastoma, suppression of tumorigenicity 1; insulin-like growth factor binding protein 2, 36 kDa; Homo sapiens cDNA FLJ12280 fis, clone MAMMA1001744; cytokine receptor-like factor 1; potassium intermediate/small conductance calcium-activated channel, subfamily N, member 4; integrin, beta 7; transcriptional co-activator with PDZ-binding motif (T AZ); sine oculis homeobox homolog 2 (Drosophila); KIAA1034 protein; vesicle-associated membrane protein 5 (myobrevin); EGF-containing fibulin-like extracellular matrix protein 1; early growth response 3; distal-less homeo box 5; hypothetical protein F1120373; aldo-keto reductase family 1, member C3 (3-alpha hydroxysteroid dehydrogenase, type II); biglycan; transcriptional co-activator with PDZ-binding motif (TAZ); fibronectin 1; proenkephalin; integrin, beta-like 1 (with EGF-like repeat domains); Homo sapiens mRNA full length insert cDNA clone EUROIMAGE 1968422; EphA3; KIAA0367 protein; natriuretic peptide receptor C/guanylate cyclase C (atrionatriuretic peptide receptor C); hypothetical protein FLJ14054; Homo sapiens mRNA; cDNA DKFZp564B222 (from clone DKFZp564B222); BCL2/adenovirus E1B 19 kDa interacting protein 3-like; AE binding protein 1; cytochrome c oxidase subunit VIIa polypeptide 1 (muscle); similar to neuralin 1; B cell translocation gene 1; hypothetical protein FLJ23191; and DKFZp586L151; and (h) lack expression of hTERT or telomerase. In one embodiment, the umbilical cord tissue-derived cell further has the characteristics of (i) secretion of at least one of MCP-1, IL-6, IL-8, GCP-2, HGF, KGF, FGF, HB-EGF, BDNF, TPO, MIP1b, 1309, MDC, RANTES, and TIMP1; (j) lack of secretion of at least one of TGF-beta2, MIPla, ANG2, PDGFbb, and VEGF, as detected by ELISA. In another embodiment, the placenta tissue-derived cell further has the characteristics of (i) secretion of at least one of MCP-1, IL-6, IL-8, GCP-2, HGF, KGF, HB-EGF, BDNF, TPO, MIPla, RANTES, and TIMP1; (j) lack of secretion of at least one of TGF-beta2, ANG2, PDGFbb, FGF, and VEGF, as detected by ELISA.

In specific embodiments, the postpartum-derived cell has all the identifying features of cell type UMB 022803 (P7) (ATCC Accession No. PTA-6067); cell type UMB 022803 (P17) (ATCC Accession No. PTA-6068), cell type PLA 071003 (P8) (ATCC Accession No. PTA-6074); cell type PLA 071003 (P11) (ATCC Accession No. PTA-6075); or cell type PLA 071003 (P16) (ATCC Accession No. PTA-6079. In an embodiment, the postpartum-derived cell derived from umbilicus tissue has all the identifying features of cell type UMB 022803 (P7) (ATCC Accession No. PTA-6067) or cell type UMB 022803 (P17) (ATCC Accession No. PTA-6068). In another embodiment, the postpartum-derived cell derived from placenta tissue has all the identifying features of cell type PLA 071003 (P8) (ATCC Accession No. PTA-6074); cell type PLA 071003 (P11) (ATCC Accession No. PTA-6075); or cell type PLA 071003 (P16) (ATCC Accession No. PTA-6079).

In certain embodiments, postpartum-derived cells are isolated in the presence of one or more enzyme activities comprising metalloprotease activity, mucolytic activity and neutral protease activity. Preferably, the cells have a normal karyotype, which is maintained as the cells are passaged in culture. In preferred embodiments, the postpartum-derived cells comprise each of CD10, CD13, CD44, CD73, CD90. In some embodiments, the postpartum-derived cells comprise each of CD10, CD13, CD44, CD73, CD90, PDGFr-alpha, and HLA-A,B,C. In preferred embodiments, the postpartum-derived cells do not comprise any of CD31, CD34, CD45, CD117. In some embodiments, the postpartum-derived cells do not comprise any of CD31, CD34, CD45, CD117, CD141, or HLA-DR,DP,DQ, as detected by flow cytometry. In embodiments, the cells lack expression of hTERT or telomerase.

In embodiments of the invention, the cell population is a substantially homogeneous population of postpartum-derived cells. In a specific embodiment, the population is a homogeneous population of postpartum-derived cells. In embodiments of the invention, the postpartum-derived cells are derived from human umbilical cord tissue or placental tissue substantially free of blood.

In certain embodiments, the population of postpartum-derived cells or a conditioned medium generated from a population of postpartum-derived cells as described above is administered with at least one other cell type, such as an astrocyte, oligodendrocyte, neuron, neural progenitor, neural stem cell, retinal epithelial stem cell, corneal epithelial stem cell, or other multipotent or pluripotent stem cell. In these embodiments, the other cell type can be administered simultaneously with, or before, or after, the cell population or the conditioned medium.

Likewise, in these and other embodiments, the population of postpartum-derived cells or the conditioned media prepared from the population of cells as described above is administered with at least one other agent, such as a drug for ocular therapy, or another beneficial adjunctive agent such as an anti-inflammatory agent, anti-apoptotic agents, antioxidants or growth factors. In these embodiments, the other agent can be administered simultaneously with, before, or after, the cell population or the conditioned media.

In various embodiments, the population of postpartum-derived cells or conditioned media generated from postpartum-derived cells (umbilical or placental) is administered to the surface of an eye, or is administered to the interior of an eye or to a location in proximity to the eye (e.g., behind the eye). The population of postpartum-derived cells or the conditioned media can be administered through a cannula or from a device implanted in the patient's body within or in proximity to the eye, or may be administered by implantation of a matrix or scaffold with the postpartum-derived cell population or conditioned media.

Another aspect of the invention features a composition for promoting development of functional synapses in a retinal degenerative condition, comprising a population of postpartum-derived cells, or conditioned media prepared from a population of cells, in an amount effective for promoting development of functional synapses. Preferably, the conditioned media is prepared from postpartum-derived cells as described above. More preferably, the postpartum-derived cells are isolated from a postpartum umbilical cord or placenta substantially free of blood. The degenerative condition may be an acute, chronic or progressive condition.

In certain embodiments, the composition comprises at least one other cell type, such as an astrocyte, oligodendrocyte, neuron, neural progenitor, neural stem cell, retinal epithelial stem cell, corneal epithelial stem cell, or other multipotent or pluripotent stem cell. In these or other embodiments, the composition comprises at least one other agent, such as a drug for treating the ocular degenerative disorder or other beneficial adjunctive agents, e.g., anti-inflammatory agents, anti-apoptotic agents, antioxidants or growth factors.

In some embodiments, the composition is a pharmaceutical composition further comprising a pharmaceutically acceptable carrier.

In certain embodiments, the pharmaceutical composition is formulated for administration to the surface of an eye. Alternatively, they can be formulated for administration to the interior of an eye or in proximity to the eye (e.g., behind the eye). The compositions also can be formulated as a matrix or scaffold containing the postpartum-derived cells or conditioned media.

According to yet another aspect of the invention, a kit is provided for treating a patient having an ocular degenerative condition. The kit comprises a pharmaceutically acceptable carrier, a population of postpartum-derived cells or conditioned media generated from a population of postpartum-derived cells, preferably the postpartum-derived cells described above, and instructions for using the kit in a method of treating the patient. The kit may also contain one or more additional components, such as reagents and instructions for generating the conditioned medium, or a population of at least one other cell type, or one or more agents useful in the treatment of an ocular degenerative condition.

In one embodiment, the invention is a method for inducing synapse formation in retinal degeneration, the method comprising administering to a subject a population of postpartum-derived cells, or a conditioned media prepared from a population of postpartum-derived cells, in an amount effective to induce synapse formation, wherein the postpartum-derived cells are derived from human umbilical cord tissue or placental tissue substantially free of blood, and wherein the cell population is capable of expansion in culture, has the potential to differentiate into cells of at least a neural phenotype, maintains a normal karyotype upon passaging, and has the following characteristics:

a) potential for 40 population doublings in culture;

b) production of CD10, CD13, CD44, CD73, and CD90; and

c) lack of production of CD31, CD34, CD45, CD117, and CD141, and

wherein the population of postpartum-derived cells secretes trophic factors, or conditioned media prepared from a population of postpartum-derived cells contains trophic factors secreted by the cell population. In an embodiment, the trophic factors secreted by the cell population are selected from thrombospondin-1, thrombospondin-2, and thrombospondin-4. In some embodiments, the population of cells is a substantially homogeneous population. In particular embodiments, the the population of cells is homogeneous. The postpartum-derived cells are umbilical cord tissue-derived cells or placental tissue-derived cells. In embodiments, the umbilical cord tissue-derived cell population secretes MCP-1, IL-6, IL-8, GCP-2, HGF, KGF, FGF, HB-EGF, BDNF, TPO, MIP1b, 1309, MDC, RANTES, and TIMP1. Further, the umbilical cord tissue-derived cell population lacks secretion of TGF-beta2, MIP1a, ANG2, PDGFbb, and VEGF, as detected by ELISA. In another embodiment, the placental tissue-derived cell population secretes MCP-1, IL-6, IL-8, GCP-2, HGF, KGF, HB-EGF, BDNF, TPO, MIP1a, RANTES, and TIMP1. In embodiments, the placental tissue-derived cell population lacks secretion of TGF-beta2, ANG2, PDGFbb, FGF, and VEGF, as detected by ELISA. Further, the cell population lacks expression of hTERT or telomerase. In embodiments, the umbilical cord tissue-derived cell population has increased expression of genes encoding interleukin 8 and reticulon 1 relative to a human cell that is a fibroblast, a mesenchymal stem cell, or an iliac crest bone marrow cell. In embodiments, the cell population produces vimentin and alpha-smooth muscle actin.

In another embodiment, the invention is a method for inducing synapse formation in retinal degeneration, the method comprising administering to a subject a population of umbilical cord tissue-derived cells, or a conditioned media prepared from a population of human umbilical cord tissue-derived cells, in an amount effective to induce synapse formation, wherein the cells are derived from human umbilical cord tissue substantially free of blood, and wherein the cell population is capable of expansion in culture, has the potential to differentiate into cells of at least a neural phenotype, maintains a normal karyotype upon passaging, and has the following characteristics:

a) potential for 40 population doublings in culture;

b) production of CD10, CD13, CD44, CD73, and CD90; and

c) lack of production of CD31, CD34, CD45, CD117, and CD141, and

wherein the population of umbilical cord tissue-derived cells secretes trophic factors, or conditioned media prepared from a population of human umbilical cord tissue-derived cells contains trophic factors secreted by the cell population. In an embodiment, the trophic factors secreted by the cell population are selected from thrombospondin-1, thrombospondin-2, and thrombospondin-4. In an embodiment, the cell population secretes MCP-1, IL-6, IL-8, GCP-2, HGF, KGF, FGF, HB-EGF, BDNF, TPO, MIP1b, 1309, MDC, RANTES, and TIMP1. In embodiments, the cell population lacks secretion of TGF-beta2, MIPla, ANG2, PDGFbb, and VEGF, as detected by ELISA. In some embodiments, the population of cells is a substantially homogeneous population. In particular embodiments, the the population of cells is homogeneous. Further, the cell population lacks expression of hTERT or telomerase. In embodiments, the cell population has increased expression of genes encoding interleukin 8 and reticulon 1 relative to a human cell that is a fibroblast, a mesenchymal stem cell, or an iliac crest bone marrow cell. In embodiments, the cell population produces vimentin and alpha-smooth muscle actin.

In embodiments described above, the umbilicus-derived cells or placental-derived cells have one or more of the following characteristics: are positive for HLA-A,B,C; are positive for CD10, CD13, CD44, CD73, CD90; are negative for HLA-DR,DP,DQ; lack production of, or are negative for CD31, CD34, CD45, CD117, and CD141. In embodiments, the cells produce vimentin and alpha-smooth muscle actin.

In further embodiments described above, the umbilicus-derived cells have increased expression of genes encoding interleukin 8 and reticulon 1 relative to a human cell that is a fibroblast, a mesenchymal stem cell, or an iliac crest bone marrow cell. In embodiments, the umbilicus-derived cells lack expression of hTERT or telomerase.

In an embodiment of the invention described above, the retinal degeneration, retinopathy or retinal/macular disorder is age-related macular degeneration. In an alternate embodiment, the retinal degeneration, retinopathy or retinal/macular disorder is dry age-related macular degeneration.

### BRIEF DESCRIPTION OF THE DRAWINGS

FIGS. 1A-1M. hUTC induce functional synapse formation between cultured RGCs. (FIG. 1A) Schematic representation of the experimental design. Purified RGCs were either cultured alone or co-cultured with hUTC, NHDF or rat ASC in transwell inserts for 6 days. The number and function of synapses were determined by immunocytochemistry- and electrophysiology-based assays, respectively. (FIG. 1B) Representative images of RGCs stained with antibodies specific for presynaptic (Bassoon, red) and postsynaptic (Homer, green) proteins. Bottom: The inlets (white boxes) are shown in higher magnification and the co-localized synaptic puncta (merge, yellow) are marked with white arrows. Scale bar: 20 μm. Quantification of (FIG. 1C) fold increase in the number of co-localized synaptic puncta (n=59-161 cells/condition) and (FIG. 1D) synaptic density (number of synapses per unit dendritic length, n=30 cells/condition) reveal that hUTC, like ASCs, induce excitatory synapse formation between cultured RGCs. (Fold increase was calculated by normalizing the number of synapses per cell with the number of synapses per cell in RGCs alone condition). FIGS. 1E-1I show induced synapses are electrophysiologically functional. (FIG. 1E) Example traces from whole-cell patch clamp recordings showing mEPSCs. (FIG. 1F) Cumulative probability plots of inter-event interval and (FIG. 1G) quantification of mean frequency of mEPSCs revealed that co-culture with hUTC or ASC induced increases in the number of synaptic events. (FIG. 1H) Cumulative probability plot of mEPSC amplitudes demonstrated an increase in large amplitude events in RGCs that were co-cultured with hUTC or ASC when compared to RGCs cultured alone. (FIG. 1I) The mean values of mEPSC amplitudes were not different between conditions. (For electrophysiology experiments, n=15 cells/condition. All data were expressed as mean±SEM, and significance was demonstrated as ***p<0.0001, **p<0.001, and *p<0.05, n.s. not significant.). FIGS. 1J-1M show electrophysiological waveform properties of hUTC and ASC co-culture induced synapses. The mEPSCs recorded from RGCs co-cultured with ASC or hUTC compared to CTR (RGC alone) demonstrated increases in both (FIGS. 1J, 1K) rising tau and decay tau (FIGS. 1L, 1M). n=15 cells/condition. All data were expressed as mean±SEM, and significance was demonstrated as ***p<0.0001, **p<0.001, and *p<0.05.

FIGS. 2A-2N. hUTC-conditioned media (UCM) induce functional synapse formation between cultured RGCs. (FIG. 2A) Schematic representation of the experimental design. Purified RGCs were treated with UCM (at various concentrations) or ACM for 6 days. The number and function of synapses were determined by immunocytochemistry- and electrophysiology-based assays, respectively. (FIG. 2B) Representative images of RGCs stained with antibodies specific to presynaptic (Bassoon, red) and postsynaptic (Homer, green) proteins. Bottom: The inlets (white boxes) are shown in higher magnification and the co-localized synaptic puncta (merge, yellow) are marked with white arrows. Scale bars: 20 μm. Quantification of (FIG. 2C) fold increase in synapse numbers (n=65-157 cells/condition) and (FIG. 2D) synaptic density (number of synapses per unit dendritic length, n=30 cells/condition) revealed that UCM, like ACM, induce excitatory synapse formation between cultured RGCs. (Fold increase in synapse numbers was calculated by normalizing the number of co-localized synaptic puncta per cell with the number of synapses in RGCs alone condition. (FIG. 2E) Example traces from whole-cell patch clamp recordings showing mEPSCs from RGCs cultured alone or treated with ACM (80 μg/mL) or UCM (80 μg/mL). (FIG. 2F) Cumulative probability plots of inter-event interval and (FIG. 2G) quantification of mean frequency of mEPSCs revealed that treatment with UCM or ACM induced an increase in the number of synaptic events. (FIG. 2H) Cumulative probability plot of mEPSC amplitudes demonstrated an increase in large amplitude events when RGCs were treated with UCM or ACM compared to RGCs cultured alone. (FIG. 2I) The mean values of mEPSC amplitudes were not significantly different between conditions. (For electrophysiology experiments, n=15 cells/condition. All data were expressed as mean±SEM, and significance was demonstrated as ***p<0.0001, **p<0.001, and *p<0.05.). FIGS. 2J-2N UCM-induced changes in synapse number and electrophysiological properties. (FIG. 2J) Quantification of synapse number in RGCs treated with various concentrations of ACM and UCM. Results are presented as the fold increase in synapse number (normalized to the number of co-localized synaptic puncta number in RGC alone condition, n=29-157 cells/condition). The mEPSCs recorded from RGCs treated with ACM or UCM demonstrated an increase in both (FIGS. 2K, 2L) rising tau and (FIGS. 2M, 2N) decay tau compared to RGCs cultured alone, n=15 cells/condition). All data were expressed as mean±SEM, and significance was demonstrated as ***p<0.0001, **p<0.001, and *p<0.05.

FIGS. 3A-3J. hUTC-secreted factors promote RGC survival and neurite outgrowth. (FIG. 3A) Representative images of RGCs treated with survival assay reagents (Calcein-AM for live cells (green) and Ethidium Homodimer-1 for dead cells (red)). Scale bar: 100 μm. (FIG. 3B) Quantification of RGC survival in the presence of various concentrations of ACM and UCM compared to RGCs cultured in minimal media (CTR). (n=9-10 microscopic field/condition) (FIG. 3C) The UCM-mediated survival effects are forskolin-dependent at all concentrations of UCM tested (n=9-18 microscopic field/condition). The survival effect of UCM (40 ng/mL) is additive to that of (FIG. 3D) BDNF and (FIG. 3E) CNTF. (n=18-20 microscopic field/condition) (FIG. 3F) Representative skeletonized traces of RGCs either cultured alone or treated with ACM or UCM. Scale bar: 100 nm. (FIG. 3G) Sholl analysis of neurite complexity demonstrated that RGCs treated with conditioned media have increased elaboration compared to RGCs cultured alone (n=20-24 cells/condition). Quantification of (FIG. 3H) total neurite outgrowth, (FIG. 3I) number of processes and (FIG. 3J) number of branches demonstrated increases when RGCs were treated with UCM or ACM compared to RGCs cultured alone (n=25-36 cells/condition). Graphs are presented as the fold increase normalized to the value of the RGC alone condition. All data were expressed as mean±SEM, and significance was demonstrated as ***p<0.0001, **p<0.001, and *p<0.05, n.s. not significant.

FIGS. 4A-4E. Characterization of the synaptogenic factors in the UCM. (FIG. 4A) Schematic representation of experimental design. Purified RGCs were treated with UCM for 6 days that was fractionated by using centrifugal concentrators with different molecular weight cut-offs. Synapse numbers were then quantified as described before. (FIG. 4B) Representative RGC images showing the co-localized synaptic puncta (presynaptic: Bassoon (red), postsynaptic: Homer (green)). Bottom: The white boxes are shown in higher magnification. White arrows indicate co-localized synaptic puncta. Scale bars: 20 μm. (FIG. 4C) Quantification of fold increase in synapse number. Fold increase was calculated by normalizing the number of co-localized synaptic puncta/cell to the values obtained in RGC alone condition (n=30-44 cells/condition). (FIG. 4D) Representative RGC images showing the co-localized synaptic puncta (white arrows, presynaptic: Bassoon (red) and postsynaptic: Homer (green)) in RGCs treated with UCM in the presence or absence of gabapentin (GBP, 32 μM). Scale bar: 20 p.m. (FIG. 4E) Quantification of fold increase in synapse number normalized to RGC alone condition (n=24-25 cells/condition). UCM-induced synaptogenesis was inhibited by GBP. All data were expressed as mean±SEM, and significance was demonstrated as ***p<0.0001, **p<0.001, and *p<0.05.

FIGS. 5A-5L. TSP1, TSP2 and TSP4 are required for hUTC-induced synaptogenesis. (FIG. 5A) Schematic presentation of experimental design for TSP-knockdown experiments (FIG. 5-7). TSP1, TSP2 and TSP4 expression were silenced by lentiviral shRNA transductions of hUTC and knockdown (KD) UCM was harvested. RGCs were treated with KD UCM for 6 days. UCM's effects on synapse formation (synapse assay), synaptic function (electrophysiology), neuronal survival (survival assay) and neurite outgrowth (outgrowth assay) were determined. (FIG. 5B) Western blot confirmation of TSP1, TS2P and TSP4 knockdown using lentiviral shRNA transductions. An unrelated hUTC-secreted protein called HEVIN was used as loading control. (FIG. 5C) Fold increase in synapse numbers in RGC-treated scrambled control UCM (SCR-CTR) and TSP-KD UCM, normalized to the RGC alone condition (n=30 cells/condition). (FIG. 5D) Fold increase in synapse numbers in RGCs treated with various TSP-KD UCM, normalized to RGC alone condition (n=76-81 cells/condition). (FIG. 5E) Fold increase in synapse numbers for RGCs treated with TSP1+2+4-KD UCM in the presence of purified TSPs normalized to RGC alone condition (n=30 cells/condition). Addition of all three TSPs (TSP1, 2 and 4) rescued the synaptogenic effects of TSP1+2+4-KD UCM. Representative images of RGCs from each of the different treatments can be found in the FIGS. 5H-5J. FIGS. 5F and 5G demonstrate Puromycin selection of lentivirus infected hUTC. (FIG. 5F) Kill curve of hUTC without lentiviral transduction in the presence of various puromycin concentrations at day 3 and 5 (n=3 microscopic field/per condition). (FIG. 5G) Representative images of hUTC with and without lentivirus infection in the presence of Puromycin (0.9 μg/mL) at day 3 and 5. Scale bar: 50 p.m. FIGS. 5H-5L show hUTC-secreted TSPs induce synapse formation between RGCs. (FIGS. 5H-5J) Representative images of RGCs showing the co-localized synaptic puncta (presynaptic: Bassoon (red), postsynaptic: Homer (green)) demonstrated that the synaptogenic activity of UCM was lost when TSP1+2+4-KD UCM was used. This loss could be partially rescued by adding purified TSP1 or TSP2 or TSP4 (150 ng/ml) into the KD UCM. Addition of all three TSPs rescued the full synaptogenic effect of the UCM. Bottom: The white boxes are shown in higher magnification. White arrows indicate co-localized synaptic puncta. Scale bar: 20 μm. (FIG. 5K) Quantification of the changes in synaptic density (number of co-localized synaptic puncta/neurite length) after treatment with various TSP-KD UCM. (FIG. 5L) Quantification of synapses formed between RGCs cultured alone (negative control) or RGCs treated with KD CTR UCM in the presence of purified TSPs (n=30 cells/condition). Addition of purified TSPs to KD CTR UCM did not lead to a further increase in the number of synapses. All data were expressed as mean±SEM, and significance was demonstrated as ***p<0.0001, **p<0.001, and *p<0.05, n.s. not significant. P FIGS. 6A-6N. TSP1, TSP2 and TSP4 are required for hUTC-induced increase in synaptic function. (FIG. 6A) Example traces from whole-cell patch clamp recordings showing mEPSCs from RGCs treated with AMC (positive control), SCR-CTR, TSP1+2+4-KD UCM or RGCs cultured alone (negative control). Silencing of TSPs in UCM (TSP1+2+4-KD) abolished both the (FIGS. 6B, 6C) frequency and (FIGS. 6D, 6E) amplitude increases that were achieved by SCR-CTR. Quantified data are demonstrated as cumulative probability plots (FIGS. 6B, 6D) and bar graphs of mean values (FIGS. 6C, E). n=15 cells/condition. (FIG. 6F) Example traces from whole-cell patch clamp recordings showing mEPSCs from RGCs treated with ACM (positive control), SCR-CTR or TSP1+2+4-KD UCM or RGCs cultured alone (negative control). Silencing of TSPs in UCM (TSP1+2+4-KD) abolished both the frequency (FIGS. 6G-6H) and amplitude (FIGS. 6I-6J) increases that were achieved by SCR-CTR. Quantified data are demonstrated as cumulative probability plots (FIG. 6G, FIG. 6I) and bar graphs of mean values (FIG. 6H, FIG. 6J). n=15 cells/condition. FIGS. 6K-6N demonstrate the effects of TSP knockdown on the waveform properties of UCM treated RGCs. (FIGS. 6K, 6L) The rising tau or the (FIGS. 6M, 6N) the decay tau were not significantly affected by silencing of TSPs expression in hUTC as demonstrated by the similar values between KD-CTR and TSP1+2+4-KD UCM treated RGC recordings (n=15 cells/condition). All data were expressed as mean±SEM, and significance was demonstrated as ***p<0.0001, **p<0.001, and *p<0.05.

FIGS. 7A-7R. TSPs are necessary for UCM-induced neurite outgrowth but not for cell survival. (FIG. 7A) Representative skeletonized traces of RGCs treated with SCR-CTR, KD-CTR or TSP1+2+4-KD UCM compared to RGC alone (negative control). Scale bar: 100 μm. TSP-silenced hUTC UCM (TSP1+2+4-KD UCM) resulted in decreased (FIG. 7B) length, (FIG. 7C) processes and (FIG. 7D) branches of total neurite outgrowth compared to both controls, SCR-CTR and KD-CTR (n=40-48 cells/condition). Silencing of TSPs in hUTC resulted in a UCM that decreased the (FIG. 7E) length of total neurite outgrowth (n=120-169 cells/condition) and (FIG. 7F) complexity shown by Sholl analysis (n=25 cells/condition). # indicates a significant reduction in total outgrowth compared to RGC alone condition (p<0.05). (FIG. 7G) Representative skeletonized traces of RGCs treated with KD-CTR or TSP1+2+4-KD UCM in the presence of purified TSPs. Scale bar: 100 μm. Addition of purified TSPs into TSP1+2+4-KD UCM restored function of UCM to enhance (FIG. 7H) total neurite growth (n=29-33 cells/condition) and (FIG. 7I) complexity (n=30 cells/condition). # indicates a significant reduction in total outgrowth compared to RGC alone condition (p<0.05). (FIG. 7J) Representative images of RGCs treated with survival assay reagents (Calcein-AM for live cells (green) and Ethidium Homodimer-1 for dead cells (red)). Scale bar: 100 μm. (FIG. 7K) Quantification of the percentage survival in RGCs treated with various TSP-KD UCM conditions (n=30 microscopic field/condition). Silencing of TSP2 or TSP4 or all three TSPs resulted in reduction in the survival promoting effect of UCM. (FIG. 7L) Addition of pure TSPs into TSP1+2+4-KD UCM did not restore the full survival effects of KD-UCM (n=20 microscopic field/condition). FIGS. 7M-7O show TSPs involvement in UCM-induced neurite outgrowth in RGCs. (FIG. 7M) Representative skeletonized traces of RGCs treated with SCR-CTR, KD-CTR and TSP-KD UCM compared to RGC alone (negative control). Scale bar: 100 μm. Knockdown of TSPs in hUTC resulted in decreased numbers of (FIG. 7N) processes and (FIG. 7O) branches (n=120-169 cells/condition). Fold changes in process and branch numbers were normalized to RGC alone control. FIGS. 7P-7R show that addition of purified TSPs rescued the neurite outgrowth function of TSP1+2+4-KD UCM. (FIG. 7P) Representative skeletonized traces of RGCs treated with KD-CTR or TSP1+2+4-KD UCM in the presence of purified TSPs. Scale bar: 100 μm. Addition of purified TSPs back to TSP1+2+4-KD UCM (FIG. 7Q) had minor effects on the number of processes, but (FIG. 7R) restored the fold change in the number of branches (n=29-33 cells/condition). All data were expressed as mean±SEM, normalized to RGC alone and significance was demonstrated as ***p<0.0001, **p<0.001, and *p<0.05.

FIGS. 8A-8J. hUTC-secreted TSPs induce neurite outgrowth in the presence of CSPG, Nogo-A or MBP. FIG. 8A. Quantification of total neurite outgrowth on coverslips coated with increasing concentrations of CSPG (n=45-79 cells/condition) or FIG. 8B. Nogo-A (n=30-37 cells/condition). Graphs are presented as the fold increase normalized to the value of the outgrowth of RGCs plated on coverslips that do not contain CSPG or Nogo-A. FIG. 8C. Representative skeletonized traces of RGCs treated with SCR-CTR, TSPs KD UCM and purified TSPs plated onto CSPG (0.05 ug/cm²) or FIG. 8D. Nogo-A (1 ug/cm²) coated coverslips. Scale bar: 100 μm. FIGS. 8E-8F. Quantification of total neurite outgrowth of RGCs plated onto FIG. 8E. CSPG (0.05 ug/cm², n=62-103 cells/condition) or FIG. 8F. Nogo-A (1 ug/cm², n=25-29 cells/condition) coated coverslips. Graphs are presented as the fold increase normalized to the value of the RGCs cultured with growth media only (RGC alone) under each culture condition. FIG. 8G. Representative skeletonized traces of RGCs treated with SCR-CTR or TSP1+2+4-KD UCM alone or supplemented with purified TSPs in the presence of MBP (10 ug/mL). Scale bar: 100 μm. FIG. 8H. Representative skeletonized traces of RGCs treated with purified TSPs in the presence of growth inhibiting substances. Scale bar: 100 μm. FIG. 8I. Quantification of total neurite outgrowth demonstrated enhancement of neurite outgrowth activity of UCM-secreted TSP2 in the presence of MBP. (n=40-54 cells/condition). Graph is presented as the fold increase normalized to the value of the RGC alone condition. FIG. 8J. Treatment of RGCs with purified TSPs is able to induce significant outgrowth in the presence of CSPG, Nogo-A or MBP (n=41-54 cells/condition). All data were expressed as mean±SEM, and significance was demonstrated as ***p<0.0001, **p<0.001, and *p<0.05, n.s. not significant. # indicates a significant reduction in total outgrowth compared to RGC alone condition (p<0.05).

Other features and advantages of the invention will be apparent from the detailed description and examples that follow.

### DETAILED DESCRIPTION

Various patents and other publications are referred to throughout the specification. Each of these publications is incorporated by reference herein, in its entirety. In the following detailed description of the illustrative embodiments, reference is made to the accompanying drawings that form a part hereof. These embodiments are described in sufficient detail to enable those skilled in the art to practice the invention, and it is understood that other embodiments may be utilized and that logical structural, mechanical, electrical, and chemical changes may be made without departing from the spirit or scope of the invention. To avoid detail not necessary to enable those skilled in the art to practice the embodiments described herein, the description may omit certain information known to those skilled in the art. The following detailed description is, therefore, not to be taken in a limiting sense, and the scope of the illustrative embodiments are defined by the appended claims.

### DEFINITIONS

Various terms used throughout the specification and claims are defined as set forth below and are intended to clarify the invention.

Stem cells are undifferentiated cells defined by the ability of a single cell both to self-renew, and to differentiate to produce progeny cells, including self-renewing progenitors, non-renewing progenitors, and terminally differentiated cells. Stem cells are also characterized by their ability to differentiate in vitro into functional cells of various cell lineages from multiple germ layers (endoderm, mesoderm and ectoderm), as well as to give rise to tissues of multiple germ layers following transplantation, and to contribute substantially to most, if not all, tissues following injection into blastocysts.

Stem cells are classified according to their developmental potential as: (1) totipotent; (2) pluripotent; (3) multipotent; (4) oligopotent; and (5) unipotent. Totipotent cells are able to give rise to all embryonic and extraembryonic cell types. Pluripotent cells are able to give rise to all embryonic cell types. Multipotent cells include those able to give rise to a subset of cell lineages, but all within a particular tissue, organ, or physiological system (for example, hematopoietic stem cells (HSC) can produce progeny that include HSC (self-renewal), blood cell-restricted oligopotent progenitors, and all cell types and elements (e.g., platelets) that are normal components of the blood). Cells that are oligopotent can give rise to a more restricted subset of cell lineages than multipotent stem cells; and cells that are unipotent are able to give rise to a single cell lineage (e.g., spermatogenic stem cells).

Stem cells are also categorized on the basis of the source from which they may be obtained. An adult stem cell is generally a multipotent undifferentiated cell found in tissue comprising multiple differentiated cell types. The adult stem cell can renew itself. Under normal circumstances, it can also differentiate to yield the specialized cell types of the tissue from which it originated, and possibly other tissue types. Induced pluripotent stem cells (iPS cells) are adult cells that are converted into pluripotent stem cells. (Takahashi et al., Cell, 2006; 126(4):663-676; Takahashi et al., Cell, 2007; 131:1-12). An embryonic stem cell is a pluripotent cell from the inner cell mass of a blastocyst-stage embryo. A fetal stem cell is one that originates from fetal tissues or membranes. A postpartum stem cell is a multipotent or pluripotent cell that originates substantially from extraembryonic tissue available after birth, namely, the placenta and the umbilical cord. These cells have been found to possess features characteristic of pluripotent stem cells, including rapid proliferation and the potential for differentiation into many cell lineages. Postpartum stem cells may be blood-derived (e.g., as are those obtained from umbilical cord blood) or non-blood-derived (e.g., as obtained from the non-blood tissues of the umbilical cord and placenta).

Embryonic tissue is typically defined as tissue originating from the embryo (which in humans refers to the period from fertilization to about six weeks of development). Fetal tissue refers to tissue originating from the fetus, which in humans refers to the period from about six weeks of development to parturition. Extraembryonic tissue is tissue associated with, but not originating from, the embryo or fetus. Extraembryonic tissues include extraembryonic membranes (chorion, amnion, yolk sac and allantois), umbilical cord and placenta (which itself forms from the chorion and the maternal decidua basalis).

Differentiation is the process by which an unspecialized (“uncommitted”) or less specialized cell acquires the features of a specialized cell, such as a nerve cell or a muscle cell, for example. A differentiated cell is one that has taken on a more specialized (“committed”) position within the lineage of a cell. The term committed, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type. De-differentiation refers to the process by which a cell reverts to a less specialized (or committed) position within the lineage of a cell. As used herein, the lineage of a cell defines the heredity of the cell, i.e. which cells it came from and what cells it can give rise to. The lineage of a cell places the cell within a hereditary scheme of development and differentiation.

In a broad sense, a progenitor cell is a cell that has the capacity to create progeny that are more differentiated than itself, and yet retains the capacity to replenish the pool of progenitors. By that definition, stem cells themselves are also progenitor cells, as are the more immediate precursors to terminally differentiated cells. When referring to the cells of the present invention, as described in greater detail below, this broad definition of progenitor cell may be used. In a narrower sense, a progenitor cell is often defined as a cell that is intermediate in the differentiation pathway, i.e., it arises from a stem cell and is intermediate in the production of a mature cell type or subset of cell types. This type of progenitor cell is generally not able to self-renew. Accordingly, if this type of cell is referred to herein, it will be referred to as a non-renewing progenitor cell or as an intermediate progenitor or precursor cell.

As used herein, the phrase “differentiates into an ocular lineage or phenotype” refers to a cell that becomes partially or fully committed to a specific ocular phenotype, including without limitation, retinal and corneal stem cells, pigment epithelial cells of the retina and iris, photoreceptors, retinal ganglia and other optic neural lineages (e.g., retinal glia, microglia, astrocytes, Mueller cells), cells forming the crystalline lens, and epithelial cells of the sclera, cornea, limbus and conjunctiva. The phrase “differentiates into a neural lineage or phenotype” refers to a cell that becomes partially or fully committed to a specific neural phenotype of the CNS or PNS, i.e., a neuron or a glial cell, the latter category including without limitation astrocytes, oligodendrocytes, Schwann cells and microglia.

The cells exemplified herein and preferred for use in the present invention are generally referred to as postpartum-derived cells (or PPDCs). They also may sometimes be referred to more specifically as umbilicus-derived cells or placenta-derived cells (UDCs or PDCs). In addition, the cells may be described as being stem or progenitor cells, the latter term being used in the broad sense. The term derived is used to indicate that the cells have been obtained from their biological source and grown or otherwise manipulated in vitro (e.g., cultured in a Growth Medium to expand the population and/or to produce a cell line). The in vitro manipulations of umbilical stem cells and placental stem cells and the unique features of the umbilicus-derived cells and placental-derived cells of the present invention are described in detail below. Cells isolated from postpartum placenta and umbilicus by other means is also considered suitable for use in the present invention. These other cells are referred to herein as postpartum cells (rather than postpartum-derived cells).

Various terms are used to describe cells in culture. Cell culture refers generally to cells taken from a living organism and grown under controlled conditions (“in culture” or “cultured”). A primary cell culture is a culture of cells, tissues, or organs taken directly from an organism(s) before the first subculture. Cells are expanded in culture when they are placed in a Growth Medium under conditions that facilitate cell growth and/or division, resulting in a larger population of the cells. When cells are expanded in culture, the rate of cell proliferation is sometimes measured by the amount of time needed for the cells to double in number. This is referred to as doubling time.

A cell line is a population of cells formed by one or more subcultivations of a primary cell culture. Each round of subculturing is referred to as a passage. When cells are subcultured, they are referred to as having been passaged. A specific population of cells, or a cell line, is sometimes referred to or characterized by the number of times it has been passaged. For example, a cultured cell population that has been passaged ten times may be referred to as a P10 culture. The primary culture, i.e., the first culture following the isolation of cells from tissue, is designated P0. Following the first subculture, the cells are described as a secondary culture (P1 or passage 1). After the second subculture, the cells become a tertiary culture (P2 or passage 2), and so on. It will be understood by those of skill in the art that there may be many population doublings during the period of passaging; therefore the number of population doublings of a culture is greater than the passage number. The expansion of cells (i.e., the number of population doublings) during the period between passaging depends on many factors, including but not limited to the seeding density, substrate, medium, growth conditions, and time between passaging.

The term Growth Medium generally refers to a medium sufficient for the culturing of PPDCs. In particular, one presently preferred medium for the culturing of the cells of the invention in comprises Dulbecco's Modified Essential Media (also abbreviated DMEM herein). Particularly preferred is DMEM-low glucose (also DMEM-LG herein) (Invitrogen, Carlsbad, Calif.). The DMEM-low glucose is preferably supplemented with 15% (v/v) fetal bovine serum (e.g. defined fetal bovine serum, Hyclone, Logan Utah), antibiotics/antimycotics ((preferably 50-100 Units/milliliter penicillin, 50-100 microgram/milliliter streptomycin, and 0-0.25 microgram/milliliter amphotericin B; Invitrogen, Carlsbad, Calif.)), and 0.001% (v/v) 2-mercaptoethanol (Sigma, St. Louis Mo.). As used in the Examples below, Growth Medium refers to DMEM-low glucose with 15% fetal bovine serum and antibiotics/antimycotics (when penicillin/streptomycin are included, it is preferably at 50 U/ml and 50 microgram/ml respectively; when penicillin/streptomycin/amphotericin are used, it is preferably at 100 U/ml, 100 microgram/ml and 0.25 microgram/ml, respectively). In some cases different growth media are used, or different supplementations are provided, and these are normally indicated in the text as supplementations to Growth Medium.

A conditioned medium is a medium in which a specific cell or population of cells has been cultured, and then removed. When cells are cultured in a medium, they may secrete cellular factors that can provide trophic support to other cells. Such trophic factors include, but are not limited to hormones, cytokines, extracellular matrix (ECM), proteins, vesicles, antibodies, and granules. The medium containing the cellular factors is the conditioned medium.

Generally, a trophic factor is defined as a substance that promotes survival, growth, differentiation, proliferation and/or maturation of a cell, or stimulates increased activity of a cell. The interaction between cells via trophic factors may occur between cells of different types. Cell interaction by way of trophic factors is found in essentially all cell types, and is a particularly significant means of communication among neural cell types. Trophic factors also can function in an autocrine fashion, i.e., a cell may produce trophic factors that affect its own survival, growth, differentiation, proliferation and/or maturation.

When referring to cultured vertebrate cells, the term senescence (also replicative senescence or cellular senescence) refers to a property attributable to finite cell cultures; namely, their inability to grow beyond a finite number of population doublings (sometimes referred to as Hayflick's limit). Although cellular senescence was first described using fibroblast-like cells, most normal human cell types that can be grown successfully in culture undergo cellular senescence. The in vitro lifespan of different cell types varies, but the maximum lifespan is typically fewer than 100 population doublings (this is the number of doublings for all the cells in the culture to become senescent and thus render the culture unable to divide). Senescence does not depend on chronological time, but rather is measured by the number of cell divisions, or population doublings, the culture has undergone.

The terms ocular, ophthalmic and optic are used interchangeably herein to define “of, or about, or related to the eye.” The term ocular degenerative condition (or disorder) is an inclusive term encompassing acute and chronic conditions, disorders or diseases of the eye, inclusive of the neural connection between the eye and the brain, involving cell damage, degeneration or loss. An ocular degenerative condition may be age-related, or it may result from injury or trauma, or it may be related to a specific disease or disorder. Acute ocular degenerative conditions include, but are not limited to, conditions associated with cell death or compromise affecting the eye including conditions arising from cerebrovascular insufficiency, focal or diffuse brain trauma, diffuse brain damage, infection or inflammatory conditions of the eye, retinal tearing or detachment, intra-ocular lesions (contusion penetration, compression, laceration) or other physical injury (e.g., physical or chemical burns). Chronic ocular degenerative conditions (including progressive conditions) include, but are not limited to, retinopathies and other retinal/macular disorders such as retinitis pigmentosa (RP), age-related macular degeneration (AMD), choroidal neovascular membrane (CNVM); retinopathies such as diabetic retinopathy, occlusive retinopathy, sickle cell retinopathy and hypertensive retinopathy, central retinal vein occlusion, stenosis of the carotid artery, optic neuropathies such as glaucoma and related syndromes; disorders of the lens and outer eye, e.g., limbal stem cell deficiency (LSCD), also referred to as limbal epithelial cell deficiency (LECD), such as occurs in chemical or thermal injury, Steven-Johnson syndrome, contact lens-induced keratopathy, ocular cicatricial pemphigoid, congenital diseases of aniridia or ectodermal dysplasia, and multiple endocrine deficiency-associated keratitis.

The term treating (or treatment of) an ocular degenerative condition refers to ameliorating the effects of, or delaying, halting or reversing the progress of, or delaying or preventing the onset of, an ocular degenerative condition as defined herein.

The term effective amount refers to a concentration or amount of a reagent or pharmaceutical composition, such as a growth factor, differentiation agent, trophic factor, cell population or other agent, that is effective for producing an intended result, including cell growth and/or differentiation in vitro or in vivo, or treatment of ocular degenerative conditions, as described herein. With respect to growth factors, an effective amount may range from about 1 nanogram/milliliter to about 1 microgram/milliliter. With respect to PPDCs as administered to a patient in vivo, an effective amount may range from as few as several hundred or fewer, to as many as several million or more. In specific embodiments, an effective amount may range from 10³ to 11¹¹, more specifically at least about 10⁴ cells. It will be appreciated that the number of cells to be administered will vary depending on the specifics of the disorder to be treated, including but not limited to size or total volume/surface area to be treated, as well as proximity of the site of administration to the location of the region to be treated, among other factors familiar to the medicinal biologist.

The terms effective period (or time) and effective conditions refer to a period of time or other controllable conditions (e.g., temperature, humidity for in vitro methods), necessary or preferred for an agent or pharmaceutical composition to achieve its intended result.

The term patient or subject refers to animals, including mammals, preferably humans, who are treated with the pharmaceutical compositions or in accordance with the methods described herein.

The term pharmaceutically acceptable carrier (or medium), which may be used interchangeably with the term biologically compatible carrier or medium, refers to reagents, cells, compounds, materials, compositions, and/or dosage forms that are not only compatible with the cells and other agents to be administered therapeutically, but also are, within the scope of sound medical judgment, suitable for use in contact with the tissues of human beings and animals without excessive toxicity, irritation, allergic response, or other complication commensurate with a reasonable benefit/risk ratio.

Several terms are used herein with respect to cell replacement therapy. The terms autologous transfer, autologous transplantation, autograft and the like refer to treatments wherein the cell donor is also the recipient of the cell replacement therapy. The terms allogeneic transfer, allogeneic transplantation, allograft and the like refer to treatments wherein the cell donor is of the same species as the recipient of the cell replacement therapy, but is not the same individual. A cell transfer in which the donor's cells and have been histocompatibly matched with a recipient is sometimes referred to as a syngeneic transfer. The terms xenogeneic transfer, xenogeneic transplantation, xenograft and the like refer to treatments wherein the cell donor is of a different species than the recipient of the cell replacement therapy. Transplantation as used herein refers to the introduction of autologous, or allogeneic donor cell replacement therapy into a recipient.

As used herein, the term “about” when referring to a measurable value such as an amount, a temporal duration, and the like, is meant to encompass variations of between ±20% and ±0.1%, preferably ±20% or ±10%, more preferably ±5%, even more preferably ±1%, and still more preferably ±0.1% from the specified value, as such variations are appropriate to perform the disclosed methods.

### DESCRIPTION

Ocular degenerative conditions, which encompass acute, chronic and progressive disorders and diseases having divergent causes, have as a common feature the dysfunction or loss of a specific or vulnerable group of ocular cells. This commonality enables development of similar therapeutic approaches for the repair or regeneration of vulnerable, damaged or lost ocular tissue or cells, one of which is cell-based therapy. Development of cell therapy for ocular degenerative conditions has been limited to a comparatively few types of stem or progenitor cells, including ocular-derived stem cells themselves (e.g., retinal and corneal stem cells), embryonic stem cells and a few types of adult stem or progenitor cells (e.g., neural, mucosal epithelial and bone marrow stem cells). Cells isolated from the postpartum umbilical cord and placenta have been identified a significant new source of progenitor cells for this purpose. (US 2005-0037491 and US 2010-0272803). Moreover, conditioned media generated from cells isolated from the postpartum placenta and umbilical cord tissue provides another new source for treating ocular degenerative conditions. Accordingly, in its various embodiments described herein, the present invention features methods and pharmaceutical compositions for (repair and regeneration of ocular tissues), which use conditioned media from progenitor cells, such as cells isolated from postpartum umbilical cord or placenta. The invention is applicable to ocular degenerative conditions, but is expected to be particularly suitable for a number of ocular disorders for which treatment or cure has been difficult or unavailable. These include, without limitation, age-related macular degeneration, retinitis pigmentosa, diabetic and other retinopathies.

Conditioned media derived from progenitor cells, such as cells isolated from postpartum umbilical cord or placenta, in accordance with any method known in the art is expected to be suitable for use in the present invention. In one embodiment, however, the invention uses conditioned media derived from umbilical cord tissue-derived cells (hUTCs) or placental-tissue derived cells (PDCs) as defined above, which are derived from umbilical cord tissue or placenta that has been rendered substantially free of blood, preferably in accordance with the method set forth below. The hUTCs or PDCs are capable of expansion in culture and have the potential to differentiate into cells of other phenotypes. Certain embodiments feature conditioned media prepared from such progenitor cells, pharmaceutical compositions comprising the conditioned media, and methods of using the pharmaceutical compositions for treatment of patients with acute or chronic ocular degenerative conditions. The postpartum-derived cells of the present invention have been characterized by their growth properties in culture, by their cell surface markers, by their gene expression, by their ability to produce certain biochemical trophic factors, and by their immunological properties. The conditioned media derived from the postpartum-derived cells have been characterized by the trophic factors secreted by the cells.

### Preparation of Cells

The cells, cell populations and preparations comprising cell lysates, conditioned media and the like, used in the compositions and methods of the present invention are described herein, and in detail in U.S. Pat. Nos. 7,524,489, and 7,510,873, and U.S. Pub. App. No. 2005/0058634, each incorporated by reference herein. According to the methods using postpartum cells, a mammalian umbilical cord and placenta are recovered upon or shortly after termination of either a full-term or pre-term pregnancy, for example, after expulsion of after-birth. The postpartum tissue may be transported from the birth site to a laboratory in a sterile container such as a flask, beaker, culture dish, or bag. The container may have a solution or medium, including but not limited to a salt solution, such as, for example, Dulbecco's Modified Eagle's Medium (DMEM) or phosphate buffered saline (PBS), or any solution used for transportation of organs used for transplantation, such as University of Wisconsin solution or perfluorochemical solution. One or more antibiotic and/or antimycotic agents, such as but not limited to penicillin, streptomycin, amphotericin B, gentamicin, and nystatin, may be added to the medium or buffer. The postpartum tissue may be rinsed with an anticoagulant solution such as heparin-containing solution. It is preferable to keep the tissue at about 4-10° C. prior to extraction of PPDCs. It is even more preferable that the tissue not be frozen prior to extraction of PPDCs.

Isolation of PPDCs preferably occurs in an aseptic environment. The umbilical cord may be separated from the placenta by means known in the art. Alternatively, the umbilical cord and placenta are used without separation. Blood and debris are preferably removed from the postpartum tissue prior to isolation of PPDCs. For example, the postpartum tissue may be washed with buffer solution, such as but not limited to phosphate buffered saline. The wash buffer also may comprise one or more antimycotic and/or antibiotic agents, such as but not limited to penicillin, streptomycin, amphotericin B, gentamicin, and nystatin.

Postpartum tissue comprising a whole placenta or umbilical cord, or a fragment or section thereof is disaggregated by mechanical force (mincing or shear forces). In a presently preferred embodiment, the isolation procedure also utilizes an enzymatic digestion process. Many enzymes are known in the art to be useful for the isolation of individual cells from complex tissue matrices to facilitate growth in culture. Ranging from weakly digestive (e.g. deoxyribonucleases and the neutral protease, dispase) to strongly digestive (e.g. papain and trypsin), such enzymes are available commercially. A nonexhaustive list of enzymes compatible herewith includes mucolytic enzyme activities, metalloproteases, neutral proteases, serine proteases (such as trypsin, chymotrypsin, or elastase), and deoxyribonucleases. Presently preferred are enzyme activities selected from metalloproteases, neutral proteases and mucolytic activities. For example, collagenases are known to be useful for isolating various cells from tissues. Deoxyribonucleases can digest singlestranded DNA and can minimize cell clumping during isolation. Preferred methods involve enzymatic treatment with for example collagenase and dispase, or collagenase, dispase, and hyaluronidase, and such methods are provided wherein in certain preferred embodiments, a mixture of collagenase and the neutral protease dispase are used in the dissociating step. More preferred are those methods that employ digestion in the presence of at least one collagenase from Clostridium histolyticum, and either of the protease activities, dispase and thermo lysin. Still more preferred are methods employing digestion with both collagenase and dispase enzyme activities. Also preferred are methods that include digestion with a hyaluronidase activity in addition to collagenase and dispase activities. The skilled artisan will appreciate that many such enzyme treatments are known in the art for isolating cells from various tissue sources. For example, the LIBERASE™ Blendzyme 3 (Roche) series of enzyme combinations are suitable for use in the instant methods. Other sources of enzymes are known, and the skilled artisan may also obtain such enzymes directly from their natural sources. The skilled artisan is also well equipped to assess new, or additional enzymes or enzyme combinations for their utility in isolating the cells of the invention. Preferred enzyme treatments are 0.5, 1, 1.5, or 2 hours long or longer. In other preferred embodiments, the tissue is incubated at 37° C. during the enzyme treatment of the dissociation step.

In some embodiments of the invention, postpartum tissue is separated into sections comprising various aspects of the tissue, such as neonatal, neonatal/maternal, and maternal aspects of the placenta, for instance. The separated sections then are dissociated by mechanical and/or enzymatic dissociation according to the methods described herein. Cells of neonatal or maternal lineage may be identified by any means known in the art, for example, by karyotype analysis or in situ hybridization for a Y chromosome.

Isolated cells or postpartum tissue from which PPDCs grow out may be used to initiate, or seed, cell cultures. Isolated cells are transferred to sterile tissue culture vessels either uncoated or coated with extracellular matrix or ligands such as laminin, collagen (native, denatured or crosslinked), gelatin, fibronectin, and other extracellular matrix proteins. PPDCs are cultured in any culture medium capable of sustaining growth of the cells such as, but not limited to, DMEM (high or low glucose), advanced DMEM, DMEM/MCDB 201, Eagle's basal medium, Ham's F10 medium (F10), Ham's F-12 medium (F12), Iscove's modified Dulbecco's medium, Mesenchymal Stem Cell Growth Medium (MSCGM), DMEM/F12, RPMI 1640, and cellgro FREE™. The culture medium may be supplemented with one or more components including, for example, fetal bovine serum (FBS), preferably about 2-15% (v/v); equine serum (ES); human serum (HS); beta-mercaptoethanol (BME or 2-ME), preferably about 0.001% (v/v); one or more growth factors, for example, platelet-derived growth factor (PDGF), epidermal growth factor (EGF), fibroblast growth factor (FGF), vascular endothelial growth factor (VEGF), insulin-like growth factor-1 (IGF-1), leukocyte inhibitory factor (LIF) and erythropoietin; amino acids, including L-valine; and one or more antibiotic and/or antimycotic agents to control microbial contamination, such as, for example, penicillin G, streptomycin sulfate, amphotericin B, gentamicin, and nystatin, either alone or in combination. The culture medium preferably comprises Growth Medium (DMEM-low glucose, serum, BME, and an antibiotic agent).

The cells are seeded in culture vessels at a density to allow cell growth. In a preferred embodiment, the cells are cultured at about 0 to about 5 percent by volume CO₂ in air. In some preferred embodiments, the cells are cultured at about 2 to about 25 percent O₂ in air, preferably about 5 to about 20 percent 02 in air. The cells preferably are cultured at about 25 to about 40° C. and more preferably are cultured at 37° C. The cells are preferably cultured in an incubator. The medium in the culture vessel can be static or agitated, for example, using a bioreactor. PPDCs preferably are grown under low oxidative stress (e.g., with addition of glutathione, Vitamin C, Catalase, Vitamin E, N-Acetylcysteine). “Low oxidative stress”, as used herein, refers to conditions of no or minimal free radical damage to the cultured cells.

Methods for the selection of the most appropriate culture medium, medium preparation, and cell culture techniques are well known in the art and are described in a variety of sources, including Doyle et al., (eds.), 1995, CELL & TISSUE CULTURE: LABORATORY PROCEDURES, John Wiley & Sons, Chichester; and Ho and Wang (eds.), 1991, ANIMAL CELL BIOREACTORS, Butterworth-Heinemann, Boston, which are incorporated herein by reference.

After culturing the isolated cells or tissue fragments for a sufficient period of time, PPDCs will have grown out, either as a result of migration from the postpartum tissue or cell division, or both. In some embodiments of the invention, PPDCs are passaged, or removed to a separate culture vessel containing fresh medium of the same or a different type as that used initially, where the population of cells can be mitotically expanded. The cells of the invention may be used at any point between passage 0 and senescence. The cells preferably are passaged between about 3 and about 25 times, more preferably are passaged about 4 to about 12 times, and preferably are passaged 10 or 11 times. Cloning and/or subcloning may be performed to confirm that a clonal population of cells has been isolated.

In some aspects of the invention, the different cell types present in postpartum tissue are fractionated into subpopulations from which the PPDCs can be isolated. This may be accomplished using standard techniques for cell separation including, but not limited to, enzymatic treatment to dissociate postpartum tissue into its component cells, followed by cloning and selection of specific cell types, for example but not limited to selection based on morphological and/or biochemical markers; selective growth of desired cells (positive selection), selective destruction of unwanted cells (negative selection); separation based upon differential cell agglutinability in the mixed population as, for example, with soybean agglutinin; freeze-thaw procedures; differential adherence properties of the cells in the mixed population; filtration; conventional and zonal centrifugation; centrifugal elutriation (counter-streaming centrifugation); unit gravity separation; countercurrent distribution; electrophoresis; and fluorescence activated cell sorting (FACS). For a review of clonal selection and cell separation techniques, see Freshney, 1994, CULTURE OF ANIMAL CELLS: A MANUAL OF BASIC TECHNIQUES, 3rd Ed., Wiley-Liss, Inc., New York, which is incorporated herein by reference.

The culture medium is changed as necessary, for example, by carefully aspirating the medium from the dish, for example, with a pipette, and replenishing with fresh medium. Incubation is continued until a sufficient number or density of cells accumulates in the dish. The original explanted tissue sections may be removed and the remaining cells trypsinized using standard techniques or using a cell scraper. After trypsinization, the cells are collected, removed to fresh medium and incubated as above. In some embodiments, the medium is changed at least once at approximately 24 hours post-trypsinization to remove any floating cells. The cells remaining in culture are considered to be PPDCs.

PPDCs may be cryopreserved. Accordingly, in a preferred embodiment described in greater detail below, PPDCs for autologous transfer (for either the mother or child) may be derived from appropriate postpartum tissues following the birth of a child, then cryopreserved so as to be available in the event they are later needed for transplantation.

### Characteristics of Cells

The progenitor cells of the invention, such as PPDCs, may be characterized, for example, by growth characteristics (e.g., population doubling capability, doubling time, passages to senescence), karyotype analysis (e.g., normal karyotype; maternal or neonatal lineage), flow cytometry (e.g., FACS analysis), immunohistochemistry and/or immunocytochemistry (e.g., for detection of epitopes), gene expression profiling (e.g., gene chip arrays; polymerase chain reaction (for example, reverse transcriptase PCR, real time PCR, and conventional PCR)), protein arrays, protein secretion (e.g., by plasma clotting assay or analysis of PDC-conditioned medium, for example, by Enzyme Linked ImmunoSorbent Assay (ELISA)), mixed lymphocyte reaction (e.g., as measure of stimulation of PBMCs), and/or other methods known in the art.

Examples of PPDCs derived from umbilicus tissue were deposited with the American Type Culture Collection on (ATCC, 10801 University Boulevard, Manassas, Va., 20110) Jun. 10, 2004, and assigned ATCC Accession Numbers as follows: (1) strain designation UMB 022803 (P7) was assigned Accession No. PTA-6067; and (2) strain designation UMB 022803 (P17) was assigned Accession No. PTA-6068. Examples of PPDCs derived from placental tissue were deposited with the ATCC (Manassas, Va.) and assigned ATCC Accession Numbers as follows: (1) strain designation PLA 071003 (P8) was deposited Jun. 15, 2004 and assigned Accession No. PTA-6074; (2) strain designation PLA 071003 (P11) was deposited Jun. 15, 2004 and assigned Accession No. PTA-6075; and (3) strain designation PLA 071003 (P16) was deposited Jun. 16, 2004 and assigned Accession No. PTA-6079.

In various embodiments, the PPDCs possess one or more of the following growth features: (1) they require L-valine for growth in culture; (2) they are capable of growth in atmospheres containing oxygen from about 5% to at least about 20%; (3) they have the potential for at least about 40 doublings in culture before reaching senescence; and (4) they attach and expand on a coated or uncoated tissue culture vessel, wherein the coated tissue culture vessel comprises a coating of gelatin, laminin, collagen, polyomithine, vitronectin or fibronectin.

In certain embodiments the PPDCs possess a normal karyotype, which is maintained as the cells are passaged. Karyotyping is particularly useful for identifying and distinguishing neonatal from maternal cells derived from placenta. Methods for karyotyping are available and known to those of skill in the art.

In other embodiments, the PPDCs may be characterized by production of certain proteins, including: (1) production of at least one of vimentin and alpha-smooth muscle actin; and (2) production of at least one of CD10, CD13, CD44, CD73, CD90, PDGFr-alpha, PD-L2 and HLA-A,B,C cell surface markers, as detected by flow cytometry. In other embodiments, the PPDCs may be characterized by lack of production of at least one of CD31, CD34, CD45, CD80, CD86, CD117, CD141, CD178, B7-H2, HLA-G, and HLA-DR,DP,DQ cell surface markers, as detected by flow cytometry. Particularly preferred are cells that produce vimentin and alpha-smooth muscle actin.

In other embodiments, the PPDCs may be characterized by gene expression, which relative to a human cell that is a fibroblast, a mesenchymal stem cell, or an iliac crest bone marrow cell, is increased for a gene encoding at least one of interleukin 8; reticulon 1; chemokine (C--X--C motif) ligand 1 (melonoma growth stimulating activity, alpha); chemokine (C--X--C motif) ligand 6 (granulocyte chemotactic protein 2); chemokine (C--X--C motif) ligand 3; tumor necrosis factor, alpha-induced protein 3; C-type lectin superfamily member 2; Wilms tumor 1; aldehyde dehydrogenase 1 family member A2; renin; oxidized low density lipoprotein receptor 1; Homo sapiens clone IMAGE:4179671; protein kinase C zeta; hypothetical protein DKFZp564F013; downregulated in ovarian cancer 1; and Homo sapiens gene from clone DKFZp547k1113. In an embodiment, the PPDCs derived from umbilical cord tissue may be characterized by gene expression, which relative to a human cell that is a fibroblast, a mesenchymal stem cell, or an iliac crest bone marrow cell, is increased for a gene encoding at least one of interleukin 8; reticulon 1; or chemokine (C--X--C motif) ligand 3. In another embodiment, the PPDCs derived from placental tissue may be characterized by gene expression, which relative to a human cell that is a fibroblast, a mesenchymal stem cell, or an iliac crest bone marrow cell, is increased for a gene encoding at least one of renin or oxidized low density lipoprotein receptor 1.

In yet other embodiments, the PPDCs may be characterized by gene expression, which relative to a human cell that is a fibroblast, a mesenchymal stem cell, or an iliac crest bone marrow cell, is reduced for a gene encoding at least one of: short stature homeobox 2; heat shock 27 kDa protein 2; chemokine (C--X--C motif) ligand 12 (stromal cell-derived factor 1); elastin (supravalvular aortic stenosis, Williams-Beuren syndrome); Homo sapiens mRNA; cDNA DKFZp586M2022 (from clone DKFZp586M2022); mesenchyme homeo box 2 (growth arrest-specific homeo box); sine oculis homeobox homolog 1 (Drosophila); crystallin, alpha B; disheveled associated activator of morphogenesis 2; DKFZP586B2420 protein; similar to neuralin 1; tetranectin (plasminogen binding protein); src homology three (SH3) and cysteine rich domain; cholesterol 25-hydroxylase; runt-related transcription factor 3; interleukin 11 receptor, alpha; procollagen C-endopeptidase enhancer; frizzled homolog 7 (Drosophila); hypothetical gene BC008967; collagen, type VIII, alpha 1; tenascin C (hexabrachion); iroquois homeobox protein 5; hephaestin; integrin, beta 8; synaptic vesicle glycoprotein 2; neuroblastoma, suppression of tumorigenicity 1; insulin-like growth factor binding protein 2, 36 kDa; Homo sapiens cDNA FLJ12280 fis, clone MAMMA1001744; cytokine receptor-like factor 1; potassium intermediate/small conductance calcium-activated channel, subfamily N, member 4; integrin, beta 7; transcriptional co-activator with PDZ-binding motif (TAZ); sine oculis homeobox homolog 2 (Drosophila); KIAAI034 protein; vesicle-associated membrane protein 5 (myobrevin); EGF-containing fibulin-like extracellular matrix protein 1; early growth response 3; distal-less homeo box 5; hypothetical protein FLJ20373; aldo-keto reductase family 1, member C3 (3-alpha hydroxysteroid dehydrogenase, type II); biglycan; transcriptional co-activator with PDZ-binding motif (TAZ); fibronectin 1; proenkephalin; integrin, beta-like 1 (with EGF-like repeat domains); Homo sapiens mRNA full length insert cDNA clone EUROIMAGE 1968422; EphA3; KIAA0367 protein; natriuretic peptide receptor C/guanylate cyclase C (atrionatriuretic peptide receptor C); hypothetical protein FLJ14054; Homo sapiens mRNA; cDNA DKFZp564B222 (from clone DKFZp564B222); BCL2/adenovirus E1B 19 kDa interacting protein 3-like; AE binding protein 1; and cytochrome c oxidase subunit VIIa polypeptide 1 (muscle).

In other embodiments, the PPDCs derived from umbilical cord tissue may be characterized by secretion of trophic factors selected from thrombospondin-1, thrombospondin-2, and thrombospondin-4. In embodiments, the PPDCs may be characterized by secretion of at least one of MCP-1, IL-6, IL-8, GCP-2, HGF, KGF, FGF, HB-EGF, BDNF, TPO, MIP1b, 1309, RANTES, MDC, and TIMP1. In some embodiments, the PPDCs derived from umbilical cord tissue may be characterized by lack of secretion of at least one of TGF-beta2, ANG2, PDGFbb, MIP1a and VEGF, as detected by ELISA. In alternative embodiments, PPDCs derived from placenta tissue may be characteristics by secretion of at least one of MCP-1, IL-6, IL-8, GCP-2, HGF, KGF, HB-EGF, BDNF, TPO, MIP1a, RANTES, and TIMP1, and lack of secretion of at least one of TGF-beta2, ANG2, PDGFbb, FGF, and VEGF, as detected by ELISA. In further embodiments, the PPDCs lack expression of hTERT or telomerase.

In preferred embodiments, the cell comprises two or more of the above-listed growth, protein/surface marker production, gene expression or substance-secretion characteristics. More preferred are those cells comprising, three, four, or five or more of the characteristics. Still more preferred are PPDCs comprising six, seven, or eight or more of the characteristics. Still more preferred presently are those cells comprising all of above characteristics.

In particularly preferred embodiments, the cells isolated from human umbilical cord tissue substantially free of blood, which are capable of expansion in culture, lack the production of CD117 or CD45, and do not express hTERT or telomerase. In one embodiment, the cells lack production of CD117 and CD45 and, optionally, also do not express hTERT and telomerase. In another embodiment, the cells do not express hTERT and telomerase. In yet another embodiment, the cells are isolated from human umbilical cord tissue substantially free of blood, are capable of expansion in culture, lack the production of CD117 or CD45, and do not express hTERT or telomerase, and have one or more of the following characteristics: express CD10, CD13, CD44, CD73, and CD90; do not express CD31 or CD34; express, relative to a human fibroblast, mesenchymal stem cell, or iliac crest bone marrow cell, increased levels of interleukin 8 or reticulon 1; and have the potential to differentiate.

Among cells that are presently preferred for use with the invention in several of its aspects are postpartum cells having the characteristics described above and more particularly those wherein the cells have normal karyotypes and maintain normal karyotypes with passaging, and further wherein the cells express each of the markers CD10, CD13, CD44, CD73, CD90, PDGFr-alpha, and HLA-A,B,C, wherein the cells produce the immunologically-detectable proteins which correspond to the listed markers. Still more preferred are those cells which in addition to the foregoing do not produce proteins corresponding to any of the markers CD31, CD34, CD45, CD117, CD141, or HLA-DR,DP,DQ, as detected by flow cytometry. In further preferred embodiments, the cells lack expression of hTERT or telomerase.

Certain cells having the potential to differentiate along lines leading to various phenotypes are unstable and thus can spontaneously differentiate. Presently preferred for use with the invention are cells that do not spontaneously differentiate, for example along neural lines. Preferred cells, when grown in Growth Medium, are substantially stable with respect to the cell markers produced on their surface, and with respect to the expression pattern of various genes, for example as determined using an Affymetrix GENECHIP. The cells remain substantially constant, for example in their surface marker characteristics over passaging, through multiple population doublings.

However, one feature of PPDCs is that they may be deliberately induced to differentiate into various lineage phenotypes by subjecting them to differentiation-inducing cell culture conditions. Of use in treatment of certain ocular degenerative conditions, the PPDCs may be induced to differentiate into neural phenotypes using one or more methods known in the art. For instance, as exemplified herein, PPDCs may be plated on flasks coated with laminin in Neurobasal-A medium (Invitrogen, Carlsbad, Calif.) containing B27 (B27 supplement, Invitrogen), L-glutamine and Penicillin/Streptomycin, the combination of which is referred to herein as Neural Progenitor Expansion (NPE) medium. NPE media may be further supplemented with bFGF and/or EGF. Alternatively, PPDCs may be induced to differentiate in vitro by: (1) co-culturing the PPDCs with neural progenitor cells; or (2) growing the PPDCs in neural progenitor cell-conditioned medium.

Differentiation of the PPDCs into neural phenotypes may be demonstrated by a bipolar cell morphology with extended processes. The induced cell populations may stain positive for the presence of nestin. Differentiated PPDCs may be assessed by detection of nest in, TuJ1 (BIII tubulin), GFAP, tyrosine hydroxylase, GABA, 04 and/or MBP. In some embodiments, PPDCs have exhibited the ability to form three-dimensional bodies characteristic of neuronal stem cell formation of neurospheres.

### Cell Populations

Another aspect of the invention features populations of progenitor cells, such as postpartum-derived cells, or other progenitor cells. The postpartum-derived cells may be isolated from placental or umbilical tissue. In a preferred embodiment, the cell populations comprise the PPDCs described above, and these cell populations are described in the section below.

In some embodiments, the cell population is heterogeneous. A heterogeneous cell population of the invention may comprise at least about 5%, 10%, 20%, 30%, 40%, 50%, 60%, 70%, 80%, 90%, or 95% of the cell. The heterogeneous cell populations of the invention may further comprise the progenitor cells (postpartum-derived cells), or other progenitor cells, such as epithelial or neural progenitor cells, or it may further comprise fully differentiated cells.

In some embodiments, the population is substantially homogeneous, i.e., comprises substantially only PPDCs (preferably at least about 96%, 97%, 98%, 99% or more of the cells). In some embodiments, the cell population is homogeneous. In embodiments, the homogeneous cell population of the invention may comprise umbilicus- or placenta-derived cells. Homogeneous populations of umbilicus-derived cells are preferably free of cells of maternal lineage. Homogeneous populations of placenta-derived cells may be of neonatal or maternal lineage. Homogeneity of a cell population may be achieved by any method known in the art, for example, by cell sorting (e.g., flow cytometry) or by clonal expansion in accordance with known methods. Thus, preferred homogeneous PPDC populations may comprise a clonal cell line of postpartum-derived cells. Such populations are particularly useful when a cell clone with highly desirable functionality has been isolated.

Also provided herein are populations of cells incubated in the presence of one or more factors, or under conditions, that stimulate stem cell differentiation along a desired pathway (e.g., neural, epithelial). Such factors are known in the art and the skilled artisan will appreciate that determination of suitable conditions for differentiation can be accomplished with routine experimentation. Optimization of such conditions can be accomplished by statistical experimental design and analysis, for example response surface methodology allows simultaneous optimization of multiple variables, for example in a biological culture. Presently preferred factors include, but are not limited to factors, such as growth or trophic factors, demethylating agents, co-culture with neural or epithelial lineage cells or culture in neural or epithelial lineage cell-conditioned medium, as well other conditions known in the art to stimulate stem cell differentiation along these pathways (for factors useful in neural differentiation, see, e.g., Lang, K. J. D. et al., 2004, J. Neurosci. Res. 76: 184-192; Johe, K. K. et al., 1996, Genes Devel. 10: 3129-3140; Gottleib, D., 2002, Ann. Rev. Neurosci. 25: 381-407).

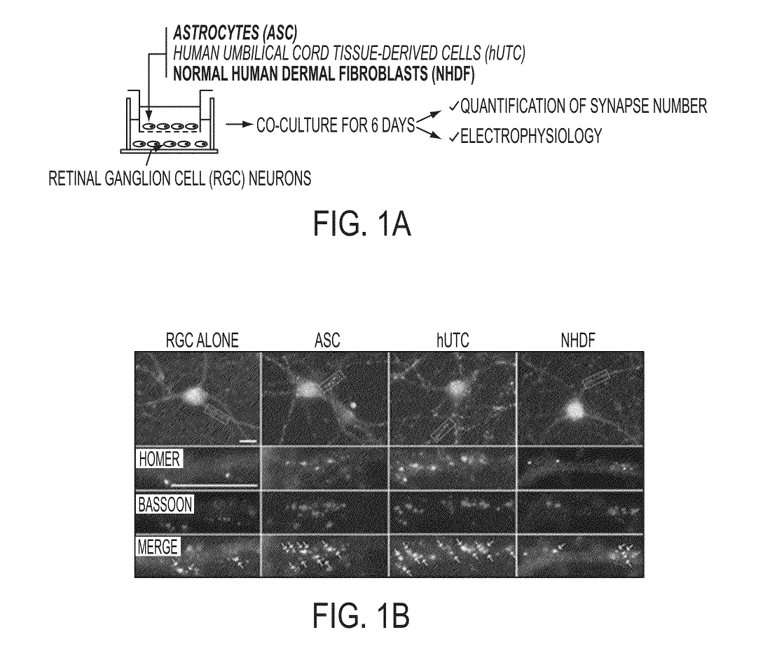

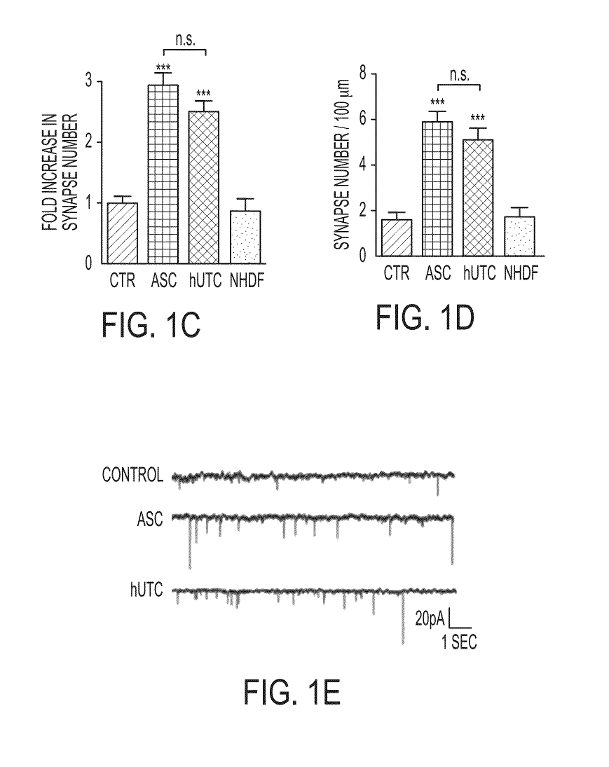

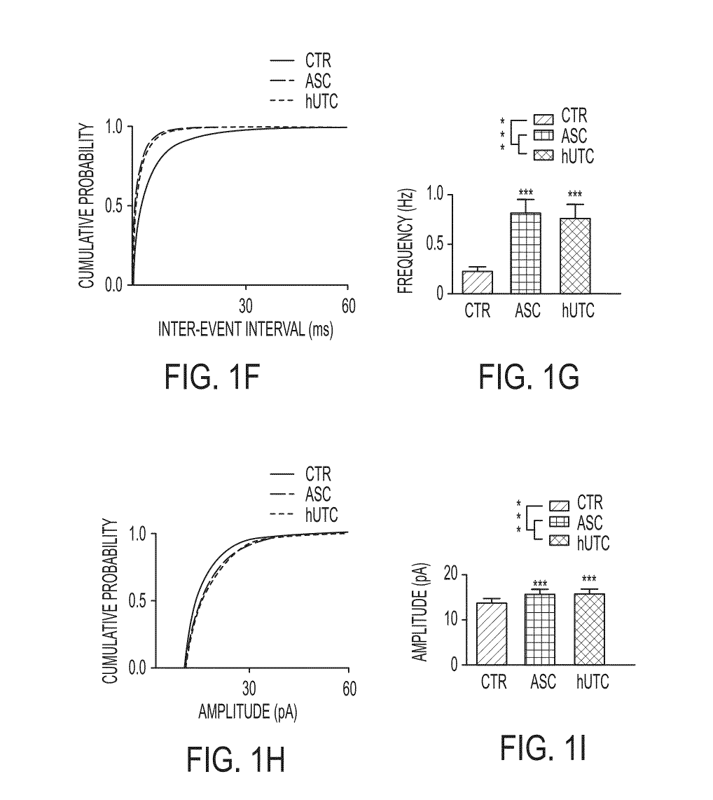

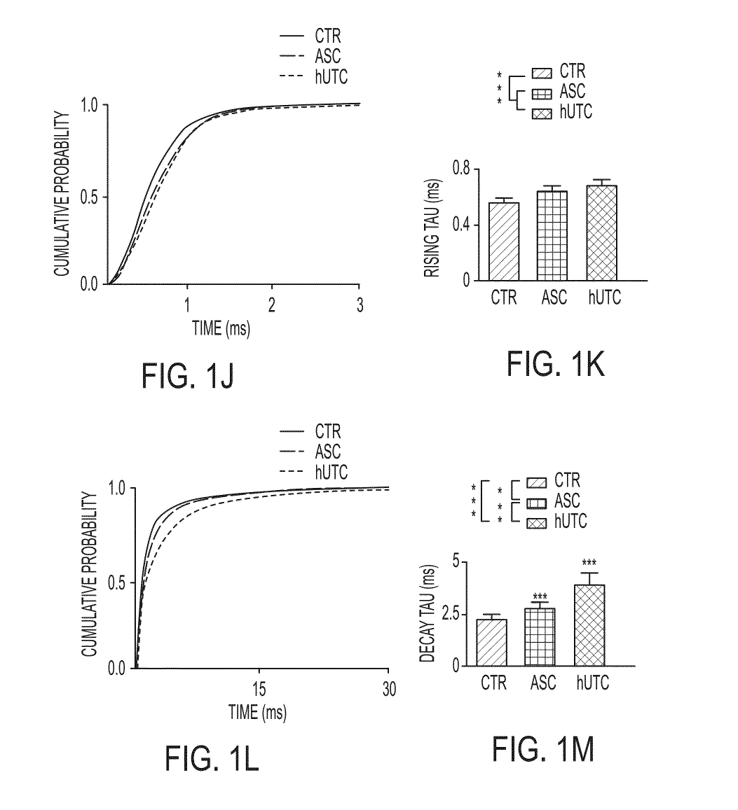

In embodiments of the invention, hUTC co-cultured with RGC had positive effects on synapse formation in RGCs, and neuronal survival and outgrowth. Co-cultures of hUTC and RGC exhibited an increase in the number of synaptic puncta and was comparable to that of astrocytes (positive control). (FIGS. 1B-1D).

Measurement of miniature excitatory postsynaptic currents (mEPSCs) show hUTC affect synaptic formation. RGCs were either cultured alone or in the presence of hUTC or ASC (FIG. 1A). Similar to astrocytes, co-culture of RGCs with hUTC led to an increase in the frequency of the synaptic events (FIG. 1E-1G, FIG. 1F, Kruskal-Wallis test, p<0.0001, FIG. 1G, One-way ANOVA, p<0.0001), which is in line with an increase in the number of synapses observed (FIG. 1B-1D). hUTC also increased the amplitude of postsynaptic currents. Like astrocytes, hUTC strengthen synaptic activity (FIGS. 1H, 1I). Waveform of mEPSC peaks revealed that both rising and decay tau are increased with hUTC treatment (FIGS. 1J-1M). hUTC induce excitatory synapse formation and enhance synaptic function in cultured RGCs.

### Conditioned Medium

In one aspect, the invention provides conditioned medium from cultured progenitor cells, such as postpartum-derived cells, or other progenitor cells, for use in vitro and in vivo as described below. Use of such conditioned medium allows the beneficial trophic factors secreted by the cells to be used allogeneically in a patient without introducing intact cells that could trigger rejection, or other adverse immunological responses. Conditioned medium is prepared by culturing cells (such as a population of cells) in a culture medium, then removing the cells from the medium. In certain embodiments, the postpartum cells are UTCs or PDCs, more preferably hUTCs.

Conditioned medium prepared from populations of cells as described above may be used as is, further concentrated, by for example, ultrafiltration or lyophilization, or even dried, partially purified, combined with pharmaceutically-acceptable carriers or diluents as are known in the art, or combined with other compounds such as biologicals, for example pharmaceutically useful protein compositions. Conditioned medium may be used in vitro or in vivo, alone or for example, with autologous or syngeneic live cells. The conditioned medium, if introduced in vivo, may be introduced locally at a site of treatment, or remotely to provide, for example needed cellular growth or trophic factors to a patient.

Previously, it has been demonstrated that human umbilical cord tissue-derived cells improved visual function and ameliorated retinal degeneration (US 2010/0272803). It also has been demonstrated that postpartum-derived cells can be used to promote photoreceptor rescue and thus preserve photoreceptors in a RCS model. (US 2010/0272803). Injection of hUTC subretinally into RCS rat eye improved visual acuity and ameliorated retinal degeneration. Moreover, treatment with conditioned medium (CM) derived from hUTC restored phagocytosis of ROS in dystrophic RPE cells in vitro. (US 2010/0272803). Here, embodiments of the invention disclose the previously unknown positiveeffect of hUTCs to promote neurite outgrowth and synaptogenesis, particularly on retinal neurons, including retinal ganglion cells, photoreceptors (rods and cones), retina amicrine cells, horizontal cells and bipolar cells.

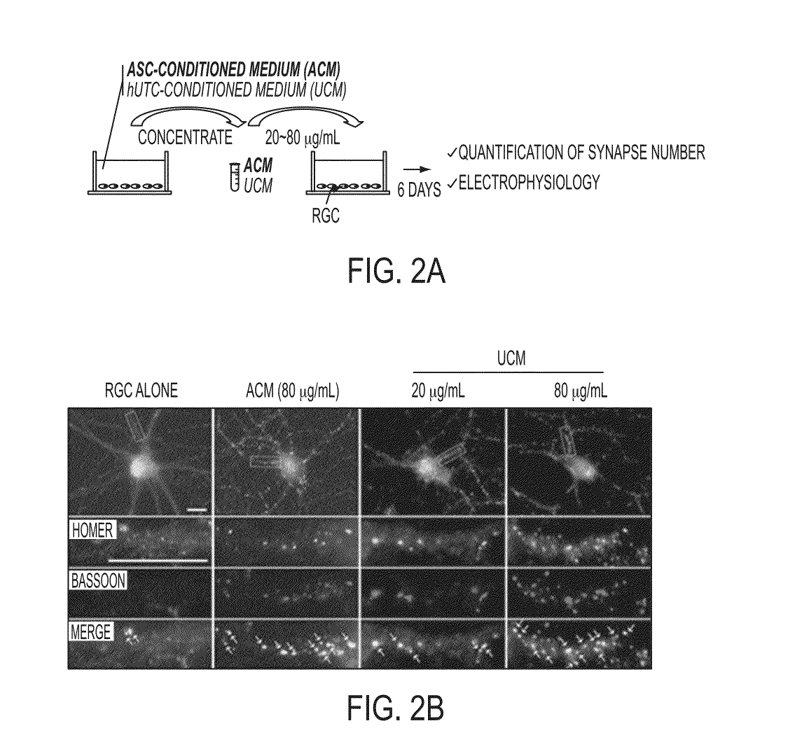

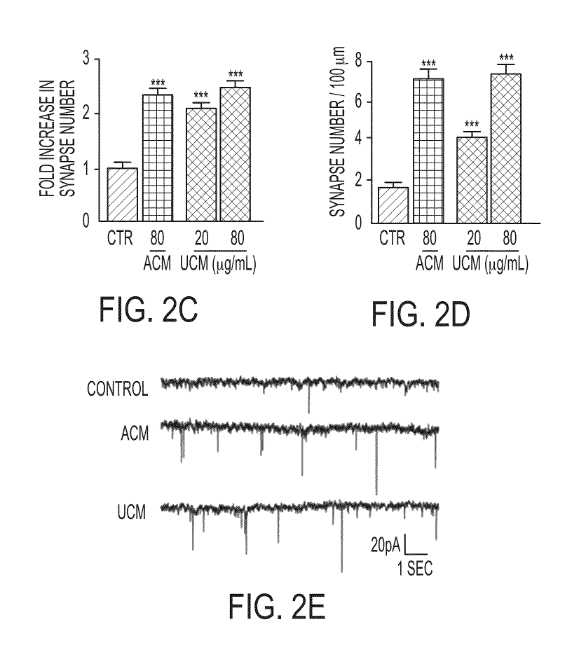

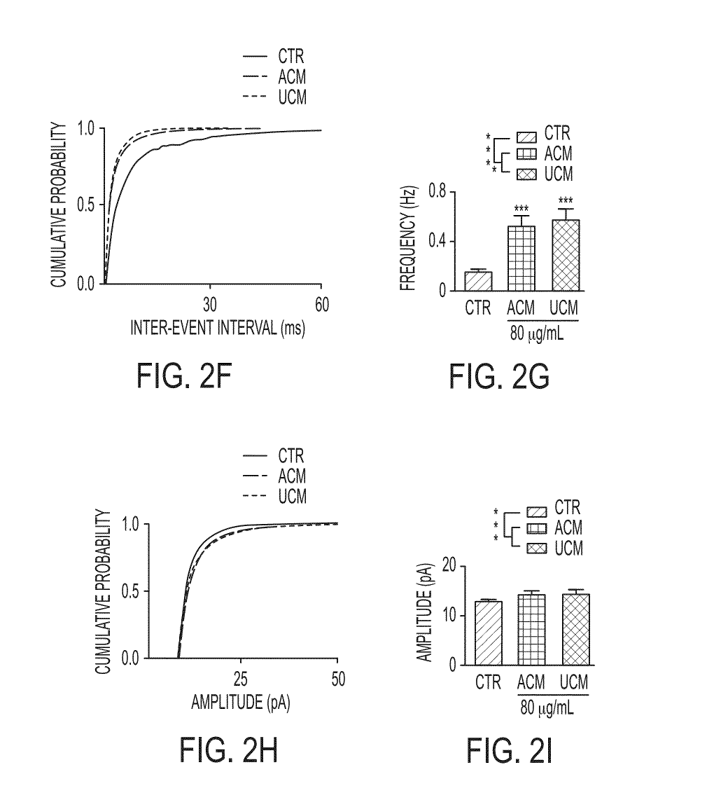

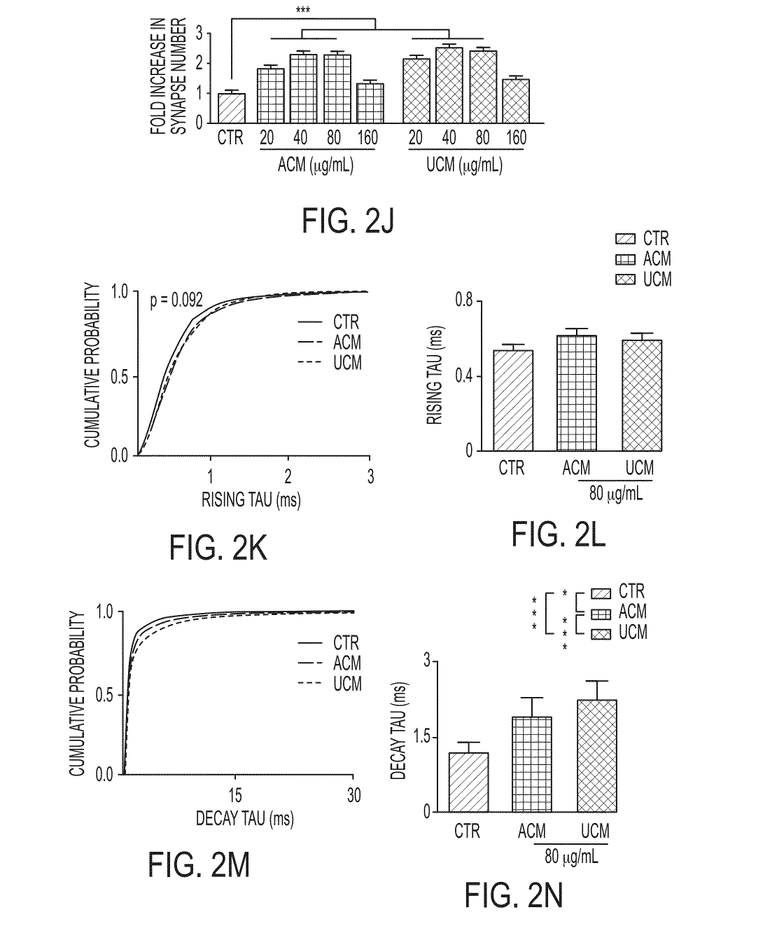

As provided herein, hUTC conditioned medium (UCM) was prepared and evaluated for the effect on synapsis formation, neuronal survival, and neurite outgrowth for retinal ganglion cells. RGCs were cultured with various concentrations of hUCM. Synapse analysis showed that hUCM induced synapse formation of RGCs in a concentration dependent manner, similar to astrocyte-conditioned media (ACM). (FIG. 2B). UCM had a positive effect on both Homer and Bassoon puncta number. (FIGS. 2B-2D, 2J).

Astrocytes have been shown to regulate synapse formation. The induction of the synapse formation is thought to be through secreted synaptogenic molecules such as thrombospondins, hevin, secreted protein acidic and rich in cysteine (SPARC), glypicans, BDNF, TGF beta-1, cholesterol and ephrins. (Clarke, Nature Reviews Neuroscience, 2013; 14:311-321; Bolton and Eroglu, Current Opinion in Neurobiology, 2009; 19:491-497). In characterizing the hUCM of the invention, astrocytes and conditioned medium prepared from astrocytes were used at times as positive controls

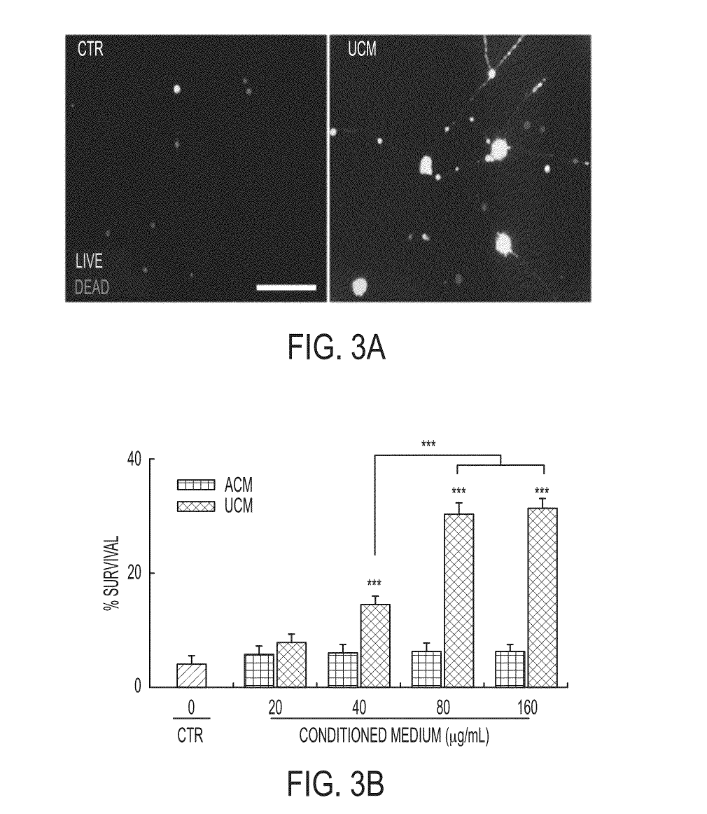

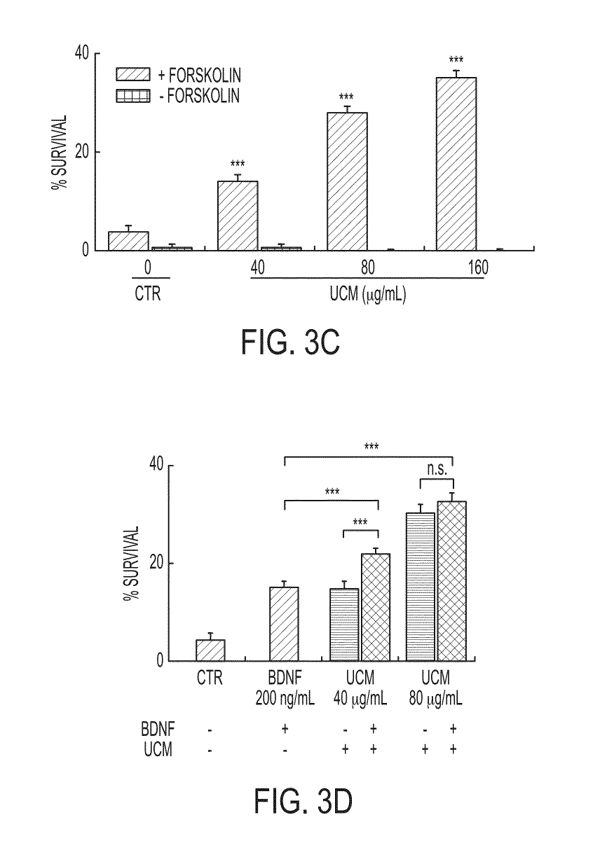

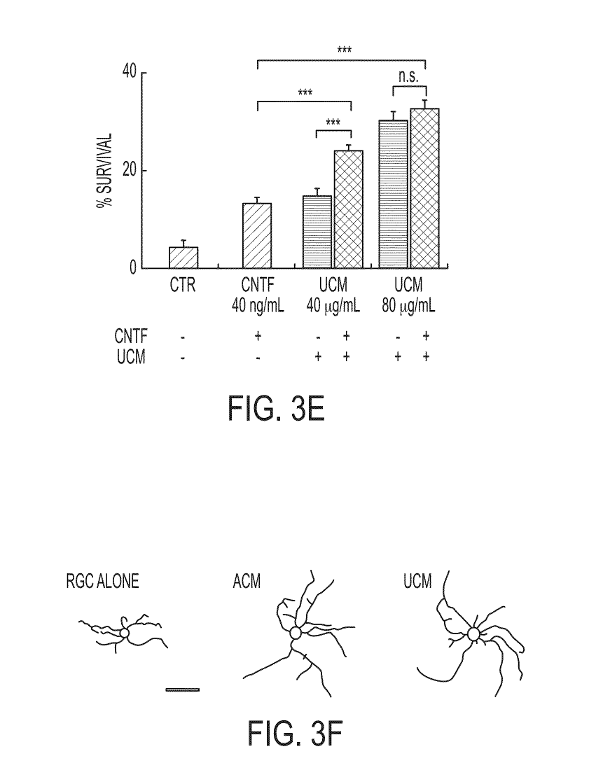

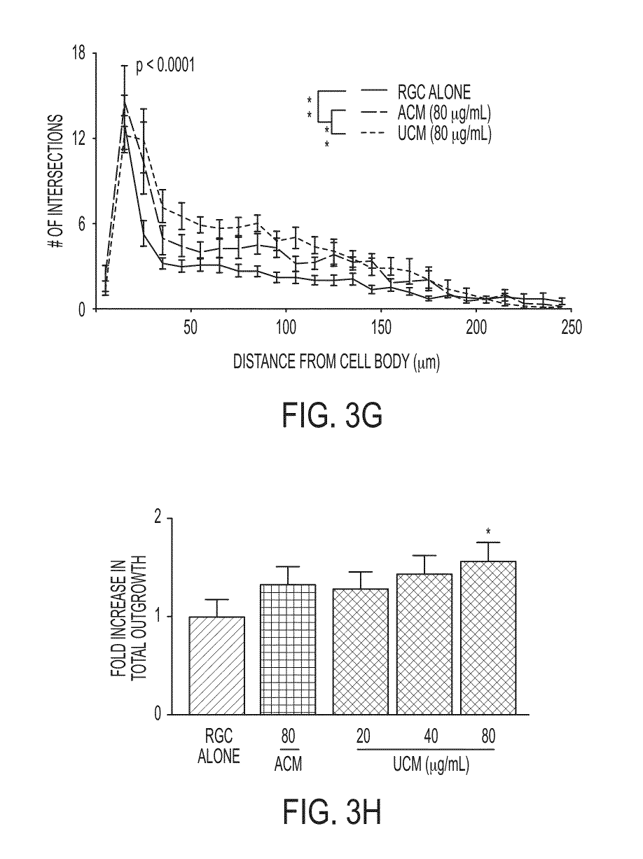

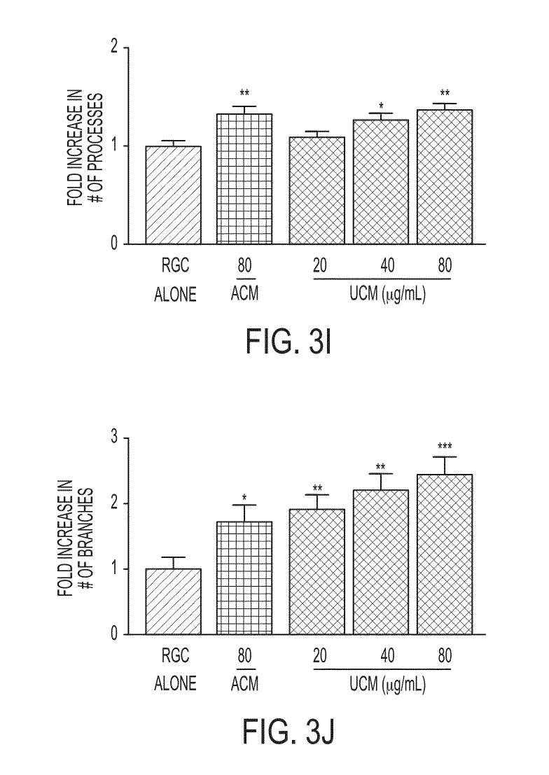

hUCM also strengthened functional synapses as shown by increased amplitude and frequency of mEPSCs. (FIGS. 2E-2N). In addition to the functional recovery that was observed after hUTC transplantations, previous studies in animal disease models revealed these cells having an effect on the preservation of the neural structures (Lund et al., 2007 supra; Moore et al., Somatosensory and motor research, 2013; 30:185-196; Zhang L, et al., Brain Research, 2012; 1489:104-112; Jiang Q, et al. PloS One, 2012; 7:e42845; Zhang L, et al., Stroke; 2011; 42:1437-1444). Using the RGC culture system, the effects of UCM are shown for synaptogenesis and neurite outgrowth. Besides its effects on synapse formation, hUCM promoted RGC survival in the absence of any other growth factors, for example, BDNF, and CTNF. (FIGS. 3A-3E).

hUCM also enhanced RGC neurite outgrowth as demonstrated by an increase in total process length, number of processes and number of branches. (FIGS. 3F-3J).

In embodiments of the invention, hUTC secrete factors that promote development of functional synapses between purified RGCs in vitro. Moreoever, hUTC also support neuronal growth and function.

### Cell Modifications, Components and Products

Progenitor cells, such as postpartum cells, preferably PPDCs, may also be genetically modified to produce therapeutically useful gene products, or to produce antineoplastic agents for treatment of tumors. Genetic modification may be accomplished using any of a variety of vectors including, but not limited to, integrating viral vectors, e.g., retrovirus vector or adeno-associated viral vectors; non-integrating replicating vectors, e.g., papilloma virus vectors, SV40 vectors, adenoviral vectors; or replication-defective viral vectors. Other methods of introducing DNA into cells include the use of liposomes, electroporation, a particle gun, or by direct DNA injection.

Hosts cells are preferably transformed or transfected with DNA controlled by or in operative association with, one or more appropriate expression control elements such as promoter or enhancer sequences, transcription terminators, polyadenylation sites, among others, and a selectable marker. Any promoter may be used to drive the expression of the inserted gene. For example, viral promoters include, but are not limited to, the CMV promoter/enhancer, SV40, papillomavirus, Epstein-Barr virus or elastin gene promoter. In some embodiments, the control elements used to control expression of the gene of interest can allow for the regulated expression of the gene so that the product is synthesized only when needed in vivo. If transient expression is desired, constitutive promoters are preferably used in a non-integrating and/or replication-defective vector. Alternatively, inducible promoters could be used to drive the expression of the inserted gene when necessary. Inducible promoters include, but are not limited to those associated with metallothionein and heat shock proteins.

Following the introduction of the foreign DNA, engineered cells may be allowed to grow in enriched media and then switched to selective media. The selectable marker in the foreign DNA confers resistance to the selection and allows cells to stably integrate the foreign DNA as, for example, on a plasmid, into their chromosomes and grow to form foci which, in turn, can be cloned and expanded into cell lines. This method can be advantageously used to engineer cell lines that express the gene product.

Cells may be genetically engineered to “knock out” or “knock down” expression of factors that promote inflammation or rejection at the implant site. Negative modulatory techniques for the reduction of target gene expression levels or target gene product activity levels are discussed below. “Negative modulation,” as used herein, refers to a reduction in the level and/or activity of target gene product relative to the level and/or activity of the target gene product in the absence of the modulatory treatment. The expression of a gene native to a neuron or glial cell can be reduced or knocked out using a number of techniques including, for example, inhibition of expression by inactivating the gene using the homologous recombination technique. Typically, an exon encoding an important region of the protein (or an exon 5′ to that region) is interrupted by a positive selectable marker, e.g., neo, preventing the production of normal mRNA from the target gene and resulting in inactivation of the gene. A gene may also be inactivated by creating a deletion in part of a gene, or by deleting the entire gene. By using a construct with two regions of homology to the target gene that are far apart in the genome, the sequences intervening the two regions can be deleted (Mombaerts el al., 1991, Proc. Nat. Acad. Sci. U.S.A. 88:3084-3087). Antisense, DNAzymes, ribozymes, small interfering RNA (siRNA) and other such molecules that inhibit expression of the target gene can also be used to reduce the level of target gene activity. For example, antisense RNA molecules that inhibit the expression of major histocompatibility gene complexes (HLA) have been shown to be most versatile with respect to immune responses. Still further, triple helix molecules can be utilized in reducing the level of target gene activity. These techniques are described in detail by L. G. Davis et al. (eds), 1994, BASIC METHODS IN MOLECULAR BIOLOGY, 2nd ed., Appleton & Lange, Norwalk, Conn.

In other aspects, the invention provides cell lysates and cell soluble fractions prepared from postpartum stem cells, preferably PPDCs, or heterogeneous or homogeneous cell populations comprising PPDCs, as well as PPDCs or populations thereof that have been genetically modified or that have been stimulated to differentiate along a neurogenic pathway. Such lysates and fractions thereof have many utilities. Use of the cell lysate soluble fraction (i.e., substantially free of membranes) in vivo, for example, allows the beneficial intracellular milieu to be used allogeneically in a patient without introducing an appreciable amount of the cell surface proteins most likely to trigger rejection, or other adverse immunological responses. Methods of lysing cells are well known in the art and include various means of mechanical disruption, enzymatic disruption, or chemical disruption, or combinations thereof. Such cell lysates may be prepared from cells directly in their Growth Medium and thus containing secreted growth factors and the like, or may be prepared from cells washed free of medium in, for example, PBS or other solution. Washed cells may be resuspended at concentrations greater than the original population density if preferred.

In one embodiment, whole cell lysates are prepared, e.g., by disrupting cells without subsequent separation of cell fractions. In another embodiment, a cell membrane fraction is separated from a soluble fraction of the cells by routine methods known in the art, e.g., centrifugation, filtration, or similar methods.

Cell lysates or cell soluble fractions prepared from populations of progenitor cells, such as postpartum-derived cells, may be used as is, further concentrated, by for example, ultrafiltration or lyophilization, or even dried, partially purified, combined with pharmaceutically-acceptable carriers or diluents as are known in the art, or combined with other compounds such as biologicals, for example pharmaceutically useful protein compositions. Cell lysates or fractions thereof may be used in vitro or in vivo, alone or for example, with autologous or syngeneic live cells. The lysates, if introduced in vivo, may be introduced locally at a site of treatment, or remotely to provide, for example needed cellular growth factors to a patient.

In a further embodiment, postpartum cells, preferably PPDCs, can be cultured in vitro to produce biological products in high yield. For example, such cells, which either naturally produce a particular biological product of interest (e.g., a trophic factor), or have been genetically engineered to produce a biological product, can be clonally expanded using the culture techniques described herein. Alternatively, cells may be expanded in a medium that induces differentiation to a desired lineage. In either case, biological products produced by the cell and secreted into the medium can be readily isolated from the conditioned medium using standard separation techniques, e.g., such as differential protein precipitation, ion-exchange chromatography, gel filtration chromatography, electrophoresis, and HPLC, to name a few. A “bioreactor” may be used to take advantage of the flow method for feeding, for example, a three-dimensional culture in vitro. Essentially, as fresh media is passed through the three-dimensional culture, the biological product is washed out of the culture and may then be isolated from the outflow, as above.

Alternatively, a biological product of interest may remain within the cell and, thus, its collection may require that the cells be lysed, as described above. The biological product may then be purified using anyone or more of the above-listed techniques.

In another embodiment, an extracellular matrix (ECM) produced by culturing postpartum cells (preferably PPDCs), on liquid, solid or semi-solid substrates is prepared, collected and utilized as an alternative to implanting live cells into a subject in need of tissue repair or replacement. The cells are cultured in vitro, on a three dimensional framework as described elsewhere herein, under conditions such that a desired amount of ECM is secreted onto the framework. The cells and the framework are removed, and the ECM processed for further use, for example, as an injectable preparation. To accomplish this, cells on the framework are killed and any cellular debris removed from the framework. This process may be carried out in a number of different ways. For example, the living tissue can be flash-frozen in liquid nitrogen without a cryopreservative, or the tissue can be immersed in sterile distilled water so that the cells burst in response to osmotic pressure.

Once the cells have been killed, the cellular membranes may be disrupted and cellular debris removed by treatment with a mild detergent rinse, such as EDTA, CHAPS or a zwitterionic detergent. Alternatively, the tissue can be enzymatically digested and/or extracted with reagents that break down cellular membranes and allow removal of cell contents. Example of such enzymes include, but are not limited to, hyaluronidase, dispase, proteases, and nucleases. Examples of detergents include non-ionic detergents such as, for example, alkylaryl polyether alcohol (TRITON X-100), octylphenoxy polyethoxy-ethanol (Rohm and Haas Philadelphia, Pa.), BRIJ-35, a polyethoxyethanollauryl ether (Atlas Chemical Co., San Diego, Calif.), polysorbate 20 (TWEEN 20), a polyethoxyethanol sorbitan mono laureate (Rohm and Haas), polyethylene lauryl ether (Rohm and Haas); and ionic detergents such as, for example, sodium dodecyl sulphate, sulfated higher aliphatic alcohols, sulfonated alkanes and sulfonated alkylarenes containing 7 to 22 carbon atoms in a branched or unbranched chain.

The collection of the ECM can be accomplished in a variety of ways, depending, for example, on whether the new tissue has been formed on a three-dimensional framework that is biodegradable or non-biodegradable. For example, if the framework is non-biodegradable, the ECM can be removed by subjecting the framework to sonication, high-pressure water jets, mechanical scraping, or mild treatment with detergents or enzymes, or any combination of the above.

If the framework is biodegradable, the ECM can be collected, for example, by allowing the framework to degrade or dissolve in solution. Alternatively, if the biodegradable framework is composed of a material that can itself be injected along with the ECM, the framework and the ECM can be processed in toto for subsequent injection. Alternatively, the ECM can be removed from the biodegradable framework by any of the methods described above for collection of ECM from a non-biodegradable framework. All collection processes are preferably designed so as not to denature the ECM.

After it has been collected, the ECM may be processed further. For example, the ECM can be homogenized to fine particles using techniques well known in the art such as by sonication, so that it can pass through a surgical needle. The components of the ECM can be crosslinked, if desired, by gamma irradiation. Preferably, the ECM can be irradiated between 0.25 to 2 mega rads to sterilize and cross link the ECM. Chemical crosslinking using agents that are toxic, such as glutaraldehyde, is possible but not generally preferred.

The amounts and/or ratios of proteins, such as the various types of collagen present in the ECM, may be adjusted by mixing the ECM produced by the cells of the invention with ECM of one or more other cell types. In addition, biologically active substances such as proteins, growth factors and/or drugs, can be incorporated into the ECM. Exemplary biologically active substances include tissue growth factors, such as TGF-beta, and the like, which promote healing and tissue repair at the site of the injection. Such additional agents may be utilized in any of the embodiments described herein above, e.g., with whole cell lysates, soluble cell fractions, or further purified components and products produced by the cells.

### Pharmaceutical Compositions

In another aspect, the invention provides pharmaceutical compositions that use non-embryronic stem cells such as postpartum cells (preferably PPDCs), cell populations thereof, conditioned media produced by such cells, and cell components and products produced by such cells in various methods for treatment of ocular degenerative conditions. Certain embodiments encompass pharmaceutical compositions comprising live cells (e.g., PPDCs alone or admixed with other cell types). Other embodiments encompass pharmaceutical compositions comprising PPDC conditioned medium. Additional embodiments may use cellular components of PPDC (e.g., cell lysates, soluble cell fractions, ECM, or components of any of the foregoing) or products (e.g., trophic and other biological factors produced naturally by the cells or through genetic modification, conditioned medium from culturing the cells). In either case, the pharmaceutical composition may further comprise other active agents, such as anti-inflammatory agents, anti-apoptotic agents, antioxidants, growth factors, neurotrophic factors or neuroregenerative, neuroprotective or ophthalmic drugs as known in the art.

Examples of other components that may be added to the pharmaceutical compositions include, but are not limited to: (1) other neuroprotective or neurobeneficial drugs; (2) selected extracellular matrix components, such as one or more types of collagen known in the art, and/or growth factors, platelet-rich plasma, and drugs (alternatively, PPDCs may be genetically engineered to express and produce growth factors); (3) anti-apoptotic agents (e.g., erythropoietin (EPO), EPO mimetibody, thrombopoietin, insulin-like growth factor (IGF)-I, IGF-II, hepatocyte growth factor, caspase inhibitors); (4) anti-inflammatory compounds (e.g., p38 MAP kinase inhibitors, TGF-beta inhibitors, statins, IL-6 and IL-1 inhibitors, PEMIROLAST, TRANILAST, REMICADE, SIROLIMUS, and non-steroidal anti-inflammatory drugs (NSAIDS) (such as TEPDXALIN, TOLMETIN, and SUPROFEN); (5) immunosuppressive or immunomodulatory agents, such as calcineurin inhibitors, mTOR inhibitors, antiproliferatives, corticosteroids and various antibodies; (6) antioxidants such as probucol, vitamins C and E, conenzyme Q-10, glutathione, L-cysteine and N-acetylcysteine; and (6) local anesthetics, to name a few.

Pharmaceutical compositions of the invention comprise progenitor cells, such as postpartum cells (preferably PPDCs), conditioned media generated from those cells, or components or products thereof, formulated with a pharmaceutically acceptable carrier or medium. Suitable pharmaceutically acceptable carriers include water, salt solution (such as Ringer's solution), alcohols, oils, gelatins, and carbohydrates, such as lactose, amylose, or starch, fatty acid esters, hydroxymethylcellulose, and polyvinyl pyrolidine. Such preparations can be sterilized, and if desired, mixed with auxiliary agents such as lubricants, preservatives, stabilizers, wetting agents, emulsifiers, salts for influencing osmotic pressure, buffers, and coloring. Typically, but not exclusively, pharmaceutical compositions comprising cellular components or products, but not live cells, are formulated as liquids. Pharmaceutical compositions comprising PPDC live cells are typically formulated as liquids, semisolids (e.g., gels) or solids (e.g., matrices, scaffolds and the like, as appropriate for ophthalmic tissue engineering).

Pharmaceutical compositions may comprise auxiliary components as would be familiar to medicinal chemists or biologists. For example, they may contain antioxidants in ranges that vary depending on the kind of antioxidant used. Reasonable ranges for commonly used antioxidants are about 0.01% to about 0.15% weight by volume of EDTA, about 0.01% to about 2.0% weight volume of sodium sulfite, and about 0.01% to about 2.0% weight by volume of sodium metabisulfite. One skilled in the art may use a concentration of about 0.1% weight by volume for each of the above. Other representative compounds include mercaptopropionyl glycine, N-acetyl cysteine, beta-mercaptoethylamine, glutathione and similar species, although other antioxidant agents suitable for ocular administration, e.g. ascorbic acid and its salts or sulfite or sodium metabisulfite may also be employed.

A buffering agent may be used to maintain the pH of eye drop formulations in the range of about 4.0 to about 8.0; so as to minimize irritation of the eye. For direct intravitreal or intraocular injection, formulations should be at pH 7.2 to 7.5, preferably at pH 7.3-7.4. The ophthalmologic compositions may also include tonicity agents suitable for administration to the eye. Among those suitable is sodium chloride to make formulations approximately isotonic with 0.9% saline solution.

In certain embodiments, pharmaceutical compositions are formulated with viscosity enhancing agents. Exemplary agents are hydroxyethylcellulose, hydroxypropylcellulose, methylcellulose, and polyvinylpyrrolidone. The pharmaceutical compositions may have cosolvents added if needed. Suitable cosolvents may include glycerin, polyethylene glycol (PEG), polysorbate, propylene glycol, and polyvinyl alcohol. Preservatives may also be included, e.g., benzalkonium chloride, benzethonium chloride, chlorobutanol, phenylmercuric acetate or nitrate, thimerosal, or methyl or propylparabens.

Formulations for injection are preferably designed for single-use administration and do not contain preservatives. Injectable solutions should have isotonicity equivalent to 0.9% sodium chloride solution (osmolality of 290-300 milliosmoles). This may be attained by addition of sodium chloride or other co-solvents as listed above, or excipients such as buffering agents and antioxidants, as listed above.

The tissues of the anterior chamber of the eye are bathed by the aqueous humor, while the retina is under continuous exposure to the vitreous. These fluids/gels exist in a highly reducing redox state because they contain antioxidant compounds and enzymes. Therefore, it may be advantageous to include a reducing agent in the ophthalmologic compositions. Suitable reducing agents include N-acetylcysteine, ascorbic acid or a salt form, and sodium sulfite or metabisulfite, with ascorbic acid and/or N-acetylcysteine or glutathione being particularly suitable for injectable solutions.

Pharmaceutical compositions comprising cells or conditioned medium, or cell components or cell products may be delivered to the eye of a patient in one or more of several delivery modes known in the art. In one embodiment that may be suitable for use in some instances, the compositions are topically delivered to the eye in eye drops or washes. In another embodiment, the compositions may be delivered to various locations within the eye via periodic intraocular injection or by infusion in an irrigating solution such as BSS or BSS PLUS (Alcon USA, Fort Worth, Tex.). Alternatively, the compositions may be applied in other ophthalmologic dosage forms known to those skilled in the art, such as pre-formed or in situ-formed gels or liposomes, for example as disclosed in U.S. Pat. No. 5,718,922 to Herrero-Vanrell. In another embodiment, the composition may be delivered to or through the lens of an eye in need of treatment via a contact lens (e.g. Lidofilcon B, Bausch & Lomb CW79 or DELTACON (Deltafilcon A) or other object temporarily resident upon the surface of the eye. In other embodiments, supports such as a collagen corneal shield (e.g. BIO-COR dissolvable corneal shields, Summit Technology, Watertown, Mass.) can be employed. The compositions can also be administered by infusion into the eyeball, either through a cannula from an osmotic pump (ALZET, Alza Corp., Palo Alto, Calif.) or by implantation of timed-release capsules (OCCUSENT) or biodegradable disks (OCULEX, OCUSERT). These routes of administration have the advantage of providing a continuous supply of the pharmaceutical composition to the eye. This may be an advantage for local delivery to the cornea.

Pharmaceutical compositions comprising live cells in a semi-solid or solid carrier are typically formulated for surgical implantation at the site of ocular damage or distress. It will be appreciated that liquid compositions also may be administered by surgical procedures, for example conditioned media. In particular embodiments, semi-solid or solid pharmaceutical compositions may comprise semi-permeable gels, lattices, cellular scaffolds and the like, which may be non-biodegradable or biodegradable. For example, in certain embodiments, it may be desirable or appropriate to sequester the exogenous cells from their surroundings, yet enable the cells to secrete and deliver biological molecules to surrounding cells. In these embodiments, cells may be formulated as autonomous implants comprising living PPDCs or cell population comprising PPDCs surrounded by a non-degradable, selectively permeable barrier that physically separates the transplanted cells from host tissue. Such implants are sometimes referred to as “immunoprotective,” as they have the capacity to prevent immune cells and macromolecules from killing the transplanted cells in the absence of pharmacologically induced immunosuppression (for a review of such devices and methods, see, e.g., P. A. Tresco et al., 2000, Adv. Drug Delivery Rev. 42: 3-27).

In other embodiments, different varieties of degradable gels and networks are utilized for the pharmaceutical compositions of the invention. For example, degradable materials particularly suitable for sustained release formulations include biocompatible polymers, such as poly (lactic acid), poly (lactic-co-glycolic acid), methylcellulose, hyaluronic acid, collagen, and the like. The structure, selection and use of degradable polymers in drug delivery vehicles have been reviewed in several publications, including, A. Domb et al., 1992, Polymers for Advanced Technologies 3:279-291. U.S. Pat. No. 5,869,079 to Wong et al. discloses combinations of hydrophilic and hydrophobic entities in a biodegradable sustained release ocular implant. In addition, U.S. Pat. No. 6,375,972 to Guo et al., U.S. Pat. No. 5,902,598 to Chen et al., U.S. Pat. No. 6,331,313 to Wong et al., U.S. Pat. No. 5,707,643 to Ogura et al., U.S. Pat. No. 5,466,233 to Weiner et al. and U.S. Pat. No. 6,251,090 to Avery et al. each describes intraocular implant devices and systems that may be used to deliver pharmaceutical compositions.

In other embodiments, e.g., for repair of neural lesions, such as a damaged or severed optic nerve, it may be desirable or appropriate to deliver the cells on or in a biodegradable, preferably bioresorbable or bioabsorbable, scaffold or matrix. These typically three-dimensional biomaterials contain the living cells attached to the scaffold, dispersed within the scaffold, or incorporated in an extracellular matrix entrapped in the scaffold. Once implanted into the target region of the body, these implants become integrated with the host tissue, wherein the transplanted cells gradually become established (see, e.g., P. A. Tresco et al., 2000, supra; see also D. W. Hutmacher, 2001, J. Biomater. Sci. Polymer Edn. 12: 107-174).

Examples of scaffold or matrix (sometimes referred to collectively as “framework”) material that may be used in the present invention include nonwoven mats, porous foams, or self-assembling peptides. Nonwoven mats may, for example, be formed using fibers comprised of a synthetic absorbable copolymer of glycolic and lactic acids (PGA/PLA), sold under the trade name VICRYL (Ethicon, Inc., Somerville, N.J). Foams, composed of, for example, poly (epsilon-caprolactone)/poly (glycolic acid) (PCL/PGA) copolymer, formed by processes such as freeze-drying, or lyophilized, as discussed in U.S. Pat. No. 6,355,699 also may be utilized. Hydrogels such as self-assembling peptides (e.g., RAD16) may also be used. In situ-forming degradable networks are also suitable for use in the invention (see, e.g., Anseth, K. S. et al., 2002, J. Controlled Release 78: 199-209; Wang, D. et al., 2003, Biomaterials 24: 3969-3980; U.S. Patent Publication 2002/0022676 to He et al.). These materials are formulated as fluids suitable for injection, and then may be induced by a variety of means (e.g., change in temperature, pH, exposure to light) to form degradable hydrogel networks in situ or in vivo.

In another embodiment, the framework is a felt, which can be composed of a multifilament yarn made from a bioabsorbable material, e.g., PGA, PLA, PCL copolymers or blends, or hyaluronic acid. The yarn is made into a felt using standard textile processing techniques consisting of crimping, cutting, carding and needling. In another embodiment, cells are seeded onto foam scaffolds that may be composite structures.

In many of the abovementioned embodiments, the framework may be molded into a useful shape. Furthermore, it will be appreciated that PPDCs may be cultured on pre-formed, non-degradable surgical or implantable devices, e.g., in a manner corresponding to that used for preparing fibroblast-containing GDC endovascular coils, for instance (Marx, W. F. et al., 2001, Am. J. Neuroradiol. 22: 323-333).

The matrix, scaffold or device may be treated prior to inoculation of cells in order to enhance cell attachment. For example, prior to inoculation, nylon matrices can be treated with 0.1 molar acetic acid and incubated in polylysine, PBS, and/or collagen to coat the nylon. Polystyrene can be similarly treated using sulfuric acid. The external surfaces of a framework may also be modified to improve the attachment or growth of cells and differentiation of tissue, such as by plasma coating the framework or addition of one or more proteins (e.g., collagens, elastic fibers, reticular fibers), glycoproteins, glycosaminoglycans (e.g., heparin sulfate, chondroitin-4-sulfate, chondroitin-6-sulfate, dermatan sulfate, keratin sulfate), a cellular matrix, and/or other materials such as, but not limited to, gelatin, alginates, agar, agarose, and plant gums, among others.

Frameworks containing living cells are prepared according to methods known in the art. For example, cells can be grown freely in a culture vessel to sub-confluency or confluency, lifted from the culture and inoculated onto the framework. Growth factors may be added to the culture medium prior to, during, or subsequent to inoculation of the cells to trigger differentiation and tissue formation, if desired. Alternatively, the frameworks themselves may be modified so that the growth of cells thereon is enhanced, or so that the risk of rejection of the implant is reduced. Thus, one or more biologically active compounds, including, but not limited to, anti-inflammatory agents, immunosuppressants or growth factors, may be added to the framework for local release.

### Methods of Use

Progenitor cells, such as postpartum cells (preferably hUTCs or PDCs), or cell populations thereof, or conditioned medium or other components of or products produced by such cells, may be used in a variety of ways to support and facilitate repair and regeneration of ocular cells and tissues. Such utilities encompass in vitro, ex vivo and in vivo methods. The methods set forth below are directed to PPDCs, but other progenitor cells may also be suitable for use in those methods.

### In Vitro and Ex Vivo Methods

In one embodiment, progenitor cells, such as postpartum cells (preferably hUTCs or PDCs), and conditioned media generated therefrom may be used in vitro to screen a wide variety of compounds for effectiveness and cytotoxicity of pharmaceutical agents, growth factors, regulatory factors, and the like. For example, such screening may be performed on substantially homogeneous populations of PPDCs to assess the efficacy or toxicity of candidate compounds to be formulated with, or co-administered with, the PPDCs, for treatment of a an ocular condition. Alternatively, such screening may be performed on PPDCs that have been stimulated to differentiate into a cell type found in the eye, or progenitor thereof, for the purpose of evaluating the efficacy of new pharmaceutical drug candidates. In this embodiment, the PPDCs are maintained in vitro and exposed to the compound to be tested. The activity of a potentially cytotoxic compound can be measured by its ability to damage or kill cells in culture. This may readily be assessed by vital staining techniques.

As discussed above, PPDCs can be cultured in vitro to produce biological products that are either naturally produced by the cells, or produced by the cells when induced to differentiate into other lineages, or produced by the cells via genetic modification. For instance, TIMP1, TPO, KGF, HGF, FGF, HBEGF, BDNF, MIP1b, MCP1, RANTES, 1309, TARC, MDC, and IL-8 were found to be secreted from umbilicus-derived cells grown in Growth Medium. Umbilicus-derived cells also secrete thrombospondin-1, thrombospondin-2, and thrombospondin-4. TIMP1, TPO, KGF, HGF, HBEGF, BDNF, MIP1a, MCP-1, RANTES, TARC, Eotaxin, and IL-8 were found to be secreted from placenta-derived PPDCs cultured in Growth Medium (see Examples).

In this regard, an embodiment of the invention features use of PPDCs for production of conditioned medium. Production of conditioned media from PPDCs may either be from undifferentiated PPDCs or from PPDCs incubated under conditions that stimulate differentiation. Such conditioned media are contemplated for use in in vitro or ex vivo culture of epithelial or neural precursor cells, for example, or in vivo to support transplanted cells comprising homogeneous populations of PPDCs or heterogeneous populations comprising PPDCs and other progenitors.

Cell lysates, soluble cell fractions or components from PPDCs, or ECM or components thereof, may be used for a variety of purposes. As mentioned above, some of these components may be used in pharmaceutical compositions. In other embodiments, a cell lysate or ECM is used to coat or otherwise treat substances or devices to be used surgically, or for implantation, or for ex vivo purposes, to promote healing or survival of cells or tissues contacted in the course of such treatments.

As described in Examples 12 and 14, PPDCs have demonstrated the ability to support survival, growth and differentiation of adult neural progenitor cells when grown in co-culture with those cells. Likewise, previous studies indicate that PPDCs may function to support cells of the retina via trophic mechanisms. (US 2010-0272803). Accordingly, PPDCs are used advantageously in co-cultures in vitro to provide trophic support to other cells, in particular neural cells and neural and ocular progenitors (e.g., neural stem cells and retinal or corneal epithelial stem cells). For co-culture, it may be desirable for the PPDCs and the desired other cells to be co-cultured under conditions in which the two cell types are in contact. This can be achieved, for example, by seeding the cells as a heterogeneous population of cells in culture medium or onto a suitable culture substrate. Alternatively, the PPDCs can first be grown to confluence, and then will serve as a substrate for the second desired cell type in culture. In this latter embodiment, the cells may further be physically separated, e.g., by a membrane or similar device, such that the other cell type may be removed and used separately, following the co-culture period. Use of PPDCs in co-culture to promote expansion and differentiation of neural or ocular cell types may find applicability in research and in clinical/therapeutic areas. For instance, PPDC co-culture may be utilized to facilitate growth and differentiation of such cells in culture, for basic research purposes or for use in drug screening assays, for example. PPDC co-culture may also be utilized for ex vivo expansion of neural or ocular progenitors for later administration for therapeutic purposes. For example, neural or ocular progenitor cells may be harvested from an individual, expanded ex vivo in co-culture with PPDCs, then returned to that individual (autologous transfer) or another individual (syngeneic or allogeneic transfer). In these embodiments, it will be appreciated that, following ex vivo expansion, the mixed population of cells comprising the PPDCs and progenitors could be administered to a patient in need of treatment. Alternatively, in situations where autologous transfer is appropriate or desirable, the co-cultured cell populations may be physically separated in culture, enabling removal of the autologous progenitors for administration to the patient.

### In Vivo Methods

As set forth in the Examples, progenitor cells (PPDCs), or conditioned media generated from such cells, may effectively be used for treating an ocular degenerative condition. Once transplanted into a target location in the eye, progenitor cells or conditioned media from progenitor cells, such as PPDCs, provide trophic support for ocular cells, including neuronal cells in situ.

Progenitor cells (PPDCs), conditioned media from progenitor cells, may be administered with other beneficial drugs, biological molecules, such as growth factors, trophic factors, conditioned medium (from progenitor or differentiated cell cultures), or other active agents, such as anti-inflammatory agents, anti-apoptotic agents, antioxidants, growth factors, neurotrophic factors or neuroregenerative or neuroprotective drugs as known in the art. When conditioned media is administered with other agents, they may be administered together in a single pharmaceutical composition, or in separate pharmaceutical compositions, simultaneously or sequentially with the other agents (either before or after administration of the other agents).

Examples of other components that may be administered with progenitor cells, such as PPDCs, and conditioned media products include, but are not limited to: (1) other neuroprotective or neurobeneficial drugs; (2) selected extracellular matrix components, such as one or more types of collagen known in the art, and/or growth factors, platelet-rich plasma, and drugs (alternatively, the cells may be genetically engineered to express and produce growth factors); (3) anti-apoptotic agents (e.g., erythropoietin (EPO), EPO mimetibody, thrombopoietin, insulin-like growth factor (IGF)-I, IGF-II, hepatocyte growth factor, caspase inhibitors); (4) anti-inflammatory compounds (e.g., p38 MAP kinase inhibitors, TGF-beta inhibitors, statins, IL-6 and IL-I inhibitors, PEMIROLAST, TRANILAST, REMICADE, SIROLIMUS, and non-steroidal anti-inflammatory drugs (NSAIDS) (such as TEPDXALIN, TOLMETIN, and SUPROFEN); (5) immunosuppressive or immunomodulatory agents, such as calcineurin inhibitors, mTOR inhibitors, antiproliferatives, corticosteroids and various antibodies; (6) antioxidants such as probucol, vitamins C and E, conenzyme Q-10, glutathione, L-cysteine and N-acetylcysteine; and (6) local anesthetics, to name a few.

Liquid or fluid pharmaceutical compositions may be administered to a more general location in the eye (e.g., topically or intra-ocularly).

Other embodiments encompass methods of treating ocular degenerative conditions by administering pharmaceutical compositions comprising conditioned medium from progenitor cells, such as PPDCs, or trophic and other biological factors produced naturally by those cells or through genetic modification of the cells. Again, these methods may further comprise administering other active agents, such as growth factors, neurotrophic factors or neuroregenerative or neuroprotective drugs as known in the art.

Dosage forms and regimes for administering conditioned media from progenitor cells, such as PPDCs, or any of the other pharmaceutical compositions described herein are developed in accordance with good medical practice, taking into account the condition of the individual patient, e.g., nature and extent of the ocular degenerative condition, age, sex, body weight and general medical condition, and other factors known to medical practitioners. Thus, the effective amount of a pharmaceutical composition to be administered to a patient is determined by these considerations as known in the art.

It may be desirable or appropriate to pharmacologically immunosuppress a patient prior to initiating cell therapy. This may be accomplished through the use of systemic or local immunosuppressive agents, or it may be accomplished by delivering the cells in an encapsulated device, as described above. These and other means for reducing or eliminating an immune response to the transplanted cells are known in the art. As an alternative, conditioned media may be prepared from PPDCs genetically modified to reduce their immunogenicity, as mentioned above.

Survival of transplanted cells in a living patient can be determined through the use of a variety of scanning techniques, e.g., computerized axial tomography (CAT or CT) scan, magnetic resonance imaging (MRI) or positron emission tomography (PET) scans. Determination of transplant survival can also be done post mortem by removing the tissue and examining it visually or through a microscope. Alternatively, cells can be treated with stains that are specific for neural or ocular cells or products thereof, e.g., neurotransmitters. Transplanted cells can also be identified by prior incorporation of tracer dyes such as rhodamine- or fluorescein-labeled microspheres, fast blue, ferric microparticles, bisbenzamide or genetically introduced reporter gene products, such as beta-galactosidase or beta-glucuronidase.

Functional integration of transplanted cells or conditioned medium into ocular tissue of a subject can be assessed by examining restoration of the ocular function that was damaged or diseased. For example, effectiveness in the treatment of macular degeneration or other retinopathies may be determined by improvement of visual acuity and evaluation for abnormalities and grading of stereoscopic color fundus photographs. (Age-Related Eye Disease Study Research Group, NEI, NIH, AREDS Report No. 8, 2001, Arch. Ophthalmol. 119: 1417-1436).

### Kits and Banks

In another aspect, the invention provides kits that utilize progenitor cells, such as PPDCs, and cell populations, conditioned medium prepared from the cells, preferably from PPDCs, and components and products thereof in various methods for ocular regeneration and repair as described above. Where used for treatment of ocular degenerative conditions, or other scheduled treatment, the kits may include one or more cell populations or conditioned medium, including at least postpartum cells or conditioned medium derived from postpartum cells, and a pharmaceutically acceptable carrier (liquid, semi-solid or solid). The kits also optionally may include a means of administering the cells and conditioned medium, for example by injection. The kits further may include instructions for use of the cells and conditioned medium. Kits prepared for field hospital use, such as for military use may include full-procedure supplies including tissue scaffolds, surgical sutures, and the like, where the cells or conditioned medium are to be used in conjunction with repair of acute injuries. Kits for assays and in vitro methods as described herein may contain, for example, one or more of: (1) PPDCs or components thereof, or conditioned medium or other products of PPDCs; (2) reagents for practicing the in vitro method; (3) other cells or cell populations, as appropriate; and (4) instructions for conducting the in vitro method.

In yet another aspect, the invention also provides for banking of tissues, cells, cell populations, conditioned medium, and cellular components of the invention. As discussed above, the cells and and conditioned medium are readily cryopreserved. The invention therefore provides methods of cryopreserving the cells in a bank, wherein the cells are stored frozen and associated with a complete characterization of the cells based on immunological, biochemical and genetic properties of the cells. The frozen cells can be thawed and expanded or used directly for autologous, syngeneic, or allogeneic therapy, depending on the requirements of the procedure and the needs of the patient. Preferably, the information on each cryopreserved sample is stored in a computer, which is searchable based on the requirements of the surgeon, procedure and patient with suitable matches being made based on the characterization of the cells or populations. Preferably, the cells of the invention are grown and expanded to the desired quantity of cells and therapeutic cell compositions are prepared either separately or as co-cultures, in the presence or absence of a matrix or support. While for some applications it may be preferable to use cells freshly prepared, the remainder can be cryopreserved and banked by freezing the cells and entering the information in the computer to associate the computer entry with the samples. Even where it is not necessary to match a source or donor with a recipient of such cells, for immunological purposes, the bank system makes it easy to match, for example, desirable biochemical or genetic properties of the banked cells to the therapeutic needs. Upon matching of the desired properties with a banked sample, the sample is retrieved and prepared for therapeutic use. Cell lysates, ECM or cellular components prepared as described herein may also be cryopreserved or otherwise preserved (e.g., by lyophilization) and banked in accordance with the present invention.

The following examples are provided to describe the invention in greater detail. They are intended to illustrate, not to limit, the invention.

The following abbreviations may appear in the examples and elsewhere in the specification and claims: ANG2 (or Ang2) for angiopoietin 2; APC for antigen-presenting cells; BDNF for brain-derived neurotrophic factor; bFGF for basic fibroblast growth factor; bid (BID) for “bis in die” (twice per day); CK18 for cytokeratin 18; CNS for central nervous system; CNTF for ciliary neurotrophic factor; CXC ligand 3 for chemokine receptor ligand 3; DMEM for Dulbecco's Minimal Essential Medium; DMEM:lg (or DMEM:Lg, DMEM:LG) for DMEM with low glucose; EDTA for ethylene diamine tetraacetic acid; EGF (or E) for epidermal growth factor; FACS for fluorescent activated cell sorting; FBS for fetal bovine serum; FGF (or F) for fibroblast growth factor; GBP for gabapentin; GCP-2 for granulocyte chemotactic protein-2; GDNF for glial cell-derived neurotrophic factor; GF AP for glial fibrillary acidic protein; HB-EGF for heparin-binding epidermal growth factor; HCAEC for Human coronary artery endothelial cells; HGF for hepatocyte growth factor; hMSC for Human mesenchymal stem cells; HNF-lalpha for hepatocyte-specific transcription factor; HVVEC for Human umbilical vein endothelial cells; 1309 for a chemokine and the ligand for the CCR8 receptor; IGF-1 for insulin-like growth factor 1; IL-6 for interleukin-6; IL-8 for interleukin 8;K19 for keratin 19; K8 for keratin 8; KGF for keratinocyte growth factor; LIF for leukemia inhibitory factor; MBP for myelin basic protein; MCP-1 for monocyte chemotactic protein 1; MDC for macrophage-derived chemokine; MIPlalpha for macrophage inflammatory protein 1 alpha; MIP lbeta for macrophage inflammatory protein 1 beta; MMP for matrix metalloprotease (MMP); MSC for mesenchymal stem cells; NHDF for Normal Human Dermal Fibroblasts; NPE for Neural Progenitor Expansion media; NT3 for neurotrophin 3; 04 for oligodendrocyte or glial differentiation marker 04; PBMC for Peripheral blood mononuclear cell; PBS for phosphate buffered saline; PDGF-CC for platelet derived growth factor C; PDGF-DD for platelet derived growth factor D; PDGFbb for platelet derived growth factor bb; PO for “per os” (by mouth); PNS for peripheral nervous system; Rantes (or RANTES) for regulated on activation, normal T cell expressed and secreted; rhGDF-5 for recombinant human growth and differentiation factor 5; SC for subcutaneously; SDF-lalpha for stromal-derived factor 1 alpha; SHH for sonic hedgehog; SOP for standard operating procedure; TARC for thymus and activation-regulated chemokine; TCP for Tissue culture plastic; TCPS for tissue culture polystyrene; TGFbeta1 for transforming growth factor beta1; TGFbeta2 for transforming growth factor beta2; TGF beta-3 for transforming growth factor beta-3; TIMP1 for tissue inhibitor of matrix metalloproteinase 1; TPO for thrombopoietin; TSP for thrombospondin; TUJ1 for BIII Tubulin; VEGF for vascular endothelial growth factor; vWF for von Willebrand factor; and alphaFP for alpha-fetoprotein.

The present invention is further illustrated, but not limited by, the following examples.

### Example 1

### Effect of Progenitor Cells on Neurite Outgrowth and Synaptogenesis

Synaptogenesis is the formation of synapses between neurons, particularly between presynaptic neurons (Bassoon) and postsynaptic neurons (Homer). This process of synapse formation is regulated by astrocytes, which along with promoting synapse formation, also provide support for neuron survival and growth, including retinal ganglion cells. It has been shown that subretinal administration of ex vivo human umbilical tissue derived cell (hUTC) to a model of retinal degeneration preserved photoreceptors and visual function. (US 2010/0272803). Here, the therapeutic aspect of hUTC is examined and the effects of derived hUTC on functional synapse formation (synaptogenesis), neuronal survival and outgrowth characterized.

### Materials and Methods

Human umbilical tissue derived cell (h UTC) were obtained from the methods described in Examples 4-16 following and in detail in U.S. Pat. Nos. 7,524,489, and 7,510,873, and U.S. Pub. App. No. 2005/0058634, both incorporated by reference herein. Briefly, human umbilical cords were obtained with donor consent following live births. Tissues were minced and enzymatically digested. After almost complete digestion with Dulbecco's modified Eagle's medium (DMEM)-low glucose (Lg) (SAFC Biosciences, Lenexa, Kans.) containing a mixture of 0.5U/mL Collagenase (Serva Elecrtrophoresis, Heidelberg, Germany), 5U/mL Neutral Protease (Serva Elecrtrophoresis, Heidelberg, Germany), and 2U/mL Hyaluronidase (Cumulase; Origio a/s, M\dot{a}aløv, Denmark), the cell suspension was filtered through a 70 μm filter, and the supernatant was centrifuged at 250×g. Isolated cells were washed in DMEM-Lg several times and plated at a density of 5,000 cells/cm′ in DMEM-Lg containing 15% (vol/vol) fetal bovine serum (FBS; SAFC Biosciences) (5% (vol/vol) carbon dioxide, 37° C.). When cells reached approximately 70% confluence (˜3-4 days), they were passaged using TrypLE (Gibco, Grand Island, N.Y.). Cells were expanded several times and banked. Cryopreserved hUTC (16-20 population doublings) were used.

Retinal Ganglion Cells: Retinal ganglion cells (RGCs) were purified by sequential immuno-panning from P7 (postnatal day 7) Sprague-Dawley rat retinas (Charles River, Wilmington, Mass.) of either sex as previously described (Winzeler A, Wang J T., Cold Spring Harbor Protocols, 2013; 643-652). Briefly, retinas were dissected and dissociated with papain (6 U/mL, Worthington, Burlingame, Calif.). Dissociated cells were panned first with Bandeiraea Simplicifolia Lectin I (BSL, Vector laboratories, Burlingame, Calif.) coated petri dishes to remove immune cells, cell debris and fibroblasts. The unbound cells were transferred to a petri dish coated with anti-Thy1 (clone T11D7) antibody for specific isolation of RGCs. The purified RGCs were gently trypsinized and re-plated onto poly-D-Lysine (PDL) and laminin-coated glass coverslips in 24-well plates (35,000 cells/coverslip). RGCs were cultured in a serum-free growth medium containing B27 (Invitrogen, Grand Island, N.Y.), brain-derived neurotrophic factor (BDNF), ciliary neurotrophic factor (CNTF), insulin and forskolin (full recipe of the media can be found in Winzeler and Wang, Cold Spring Harbor Protocols, supra).

Cortical astrocytes (ASCs) were isolated from P1 Sprague-Dawley rat pups of either sexusing standard methods as described in McCarthy K D, de Vellis J., J. Cell Biology, 1980; 85:890-902. For astrocyte co-culture, transwell inserts (BD Biosciences, Franklin Lakes, N.J.) were prepared by seeding 125,000 cortical astrocytes per insert in astrocytes growth media (AGM, described in McCarthy K D, de Vellis J., J. Cell Biology, 1980; 85:890-902). The next day AGM was replaced with RGC growth media and the transwell inserts were transferred into the 24-well plate containing the 4 DIV (days in vitro) RGCs on coverslips.

Normal human dermal fibroblast (NHDF) were obtained from Lonza (Walkersville, Md.). Cells were expanded and cryopreserved at Passage 4, according to the manufacture's protocol.

RGCs were co-cultured with hUTC, ASCs as a positive control, or NHDF cell line as a negative control for six days. hUTC at DIV3 and NHDF were seeded at 10K, 20K, 30K, 100K and 200K. Astrocytes were seeded at 130K. hUTC at 25K, 50K and 100K, Astrocytes at 130K, and NHDF at 100K, 150K and 200K were used for calculating the effect on synapse formation.

For co-culture experiments, 25,000 hUTC were seeded into transwell inserts in hUTC growth media a day prior to the co-culture with RGCs. The day of co-culture the hUTC growth media was replaced with RGCs growth media and transwell inserts were transferred into the 24-well plates with 4 DIV RGCs that are grown on glass coverslips.

NHDF were cultured in Fibroblast Basal Medium (FBM, Lonza) using manufacturer's recommendations. For co-culture with RGCs, 150,000 NHDF were seeded into transwell inserts in FBM media a day prior to the co-culture with RGCs. The next day FBM was replaced with RGC growth media and the transwell inserts were transferred into the 24-well plate containing the 4 DIV RGCs on coverslips.

Quantification of Synapse Number by Immunocytochemistry (Synapse Assay):

Synapse formation was assessed by immunocytological analyses of the co-localization of pre-(Bassoon) and post-synaptic (Homer) markers, and electrophysiology. 35,000 RGCs were cultured alone on glass coverslips for 4 DIV and transwell inserts or conditioned media were added on 4 DIV. To block TSP-induced synapse formation, 32 μM Gabapentin (GBP) was added together with the conditioned media as described in Eroglu C, et al., Cell, 2009; 139:380-392. The co-cultured cells received fresh growth media on 7 DIV. When conditioned media were used, growth media supplemented with conditioned media were provided at 7 DIV. On 10 DIV, RGCs were fixed with 4% PFA (w/v) and stained for pre- and post-synaptic markers bassoon (mouse anti-bassoon, 1:1000, RRID: AB_2038857, Enzo, Farmingdale, N.Y.) and homer-1 (rabbit anti-homer, 1:500, RRID: AB_1966438, Synaptic Systems, Goettngen, Germany). Alexa-conjugated secondary antibodies (Invitrogen, goat anti rabbit AF-488 (1:1000, RRID: AB_10563748) and goat anti mouse AF-594 (1:500, RRID: AB_10561507)) were used for detection. Coverslips were mounted in Vectashield mounting medium with DAPI (Vector Laboratories) on glass slides (VWR Scientific, Radnor, Pa.). RGCs were imaged on a Zeiss Axioimager M1 Epifluorescence Microscope (Carl Zeiss, Thornwood, N.Y.) using a 63× objective. Morphologically healthy single cells that were at least two cell diameters from their nearest neighbor were identified randomly by DAPI fluorescence. At least 15-20 cells per condition were imaged and analyzed per experiment and the results presented are the average of 2-3 independent experiments. Captured images were analyzed for co-localized synaptic puncta with a custom plug-in (written by Barry Wark, available upon request from Cagla Eroglu at Duke University) for the NIH image-processing package ImageJ. The details of the staining protocol and the quantification method are described in detail in Ippolito D M, Eroglu C., J Vis Exp., 2010; 16(45):2270. Synaptic densities (number of synapses per neurite length) were determined by counting the number of co-localized synaptic puncta per 100 μm dendrite. For this analysis one proximal dendrite/cell was randomly chosen per image. The synaptic density results presented are from one experiment with 30 cells/condition.

Electrophysiological Recordings from RGCs:

Miniature excitatory postsynaptic currents (mEPSCs) were recorded by whole-cell patch clamp at a holding potential of −70 mV. The cells were maintained under continuous perfusion of the extracellular solution at 23-24° C. with 0.2 mL/min flow rate. The extracellular solution contained: 124 mM NaCl, 2.5 mM KCl, 2 mM CaCl₂, 1 mM MgCl₂, 26 mM NaHCO3, 1.2 mM NaH₂PO₄, 10 mM D-glucose (pH 7.4). Tetrodotoxin (TTX, Abcam, 1 μM, Abcam, Cambridge, Mass.) was added in the extracellular solution during recording. The internal solution in patch pipettes (2.5 to 5 MΩ) contained: 120 mM cesium methane sulfonate, 5 mM NaCl, 10 mM tetraethylammonium chloride, 10 mM HEPES, 4 mM lidocaine N-ethyl bromide, 1.1 mM EGTA, 4 mM magnesium ATP, and 0.3 mM sodium GTP, pH adjusted to 7.2 with CsOH and osmolality set to 300 mOsm with sucrose. Signals were recorded by MultiClamp 700B amplifier (Molecular Devices, Sunnyvale, Calif.) and filtered at 10 kHz and digitized at 20 kHz with a Digidata 1440A digitizer (Molecular Devices). In the whole-cell configuration, recordings were only accepted when the series resistance is <20 MΩ. All data were analyzed using peak detection software in pCLAMP10 (Molecular Devices). Data presented is acquired from 15 cells/condition that were recorded over 3 independent experiments.

Neurite Outgrowth Assay:

RGCs were seeded onto poly-D-lysine (PDL) and mouse laminin (20 ng/ml)-coated glass coverslips at low density (1,500 cells/coverslip) for 24 hours and then were fixed with 4% PFA (w/v) and immuno-stained with rabbit anti-β Tubulin antibody (LI-COR, RRID: AB_1850029, Lincoln, Nebr.), followed with goat anti-rabbit AF-488 to visualize the neurites. After 24 hours, neurites were visualized by incubating with CellTracker Red CMPTX dye (Inivtirogen) for 15 mins followed by fixation with 4% PFA (w/v). Images of single RGCs with their neurites were captured using a 20× objective on a Zeiss Axioimager M1 Epifluorescence Microscope (Carl Zeiss). At least 30 cells per condition were imaged and analyzed per experiment and the results presented are the average of 2-3 independent experiments. Neuronal morphology was analyzed using neurite outgrowth application module in Metamorph software (Molecular Devices). Sholl analysis was performed using a plug-in for Fiji as described in Ferreira T A, et al., Nature Methods, 2014; 11:982-984.

Statistical analyses of the quantified data were done using one-way analysis of variance (ANOVA) followed by Each Pair, Student's t (Fisher's LSD) post-test. For Sholl analyses of neurite outgrowth assays, analysis of co-variance (ANCOVA) was performed. For analyses of cumulative probabilities of mEPSCs, Kruskal-Wallis test followed by Dunn's multiple comparison post-hoc test were used. JMP Genomics 5 embedded with SAS 9.2 software (SAS) was used for all statistical analysis of the data. All data was expressed as mean±SEM, and significance was demonstrated as ***p<0.0001, **p<0.001, and *p<0.05.

### Results

Changes in the number and function of synapses made between purified RGCs when they were co-cultured with hUTC in transwell inserts were found. RGCs co-cultured with primary rat astroglial cultures (ASC) provided a positive control, as astrocytes are shown to strongly increase synapse numbers and enhance synaptic activity (Pfrieger F W, Barres B A, Science, 1997; 277:1684-1687; Ullian E M, et al., Science, 2001; 291:657-661). RGCs treated with NHDF provided a negative control, since they did not induce significant functional or structural recovery in a model of retinal degeneration (Lund et al., 2007 supra). Co-culture of hUTC with RGC exhibited an increase in the number of synaptic puncta and was comparable to that of astrocytes (positive control). (FIGS. 1B-1D). After 4 DIV, RGCs were co-cultured with hUTC, ASC or NHDF for an additional 6 days and the number of synapses was assessed by immunostaining with a pair of pre- and post-synaptic proteins, bassoon and homer, respectively (FIG. 1A). Synapses were determined as the co-localization of pre- and post-synaptic markers using methods described previously (Ippolito D M, Eroglu C., J Vis Exp., 2010 supra) (FIG. 1B, arrows). The hUTC induced the formation of excitatory synapses between RGCs when compared to RGCs cultured alone (FIGS. 1B, 1C). Whereas co-culture with NHDF did not induce a significant increase in synapse number (FIGS. 1B, 1C, 1D). Quantification of the number of synapses per unit neurite length revealed that hUTC enhance synapse numbers primarily by increasing the synapse density (FIG. 1D, One-way ANOVA, p<0.0001).

Whole-cell patch clamp recordings measured the miniature excitatory postsynaptic currents (mEPSCs). RGCs were either cultured alone or in the presence of hUTC or ASC (FIG. 1A). Similar to astrocytes, co-culture of RGCs with hUTC led to an increase in the frequency of the synaptic events (FIG. 1E-1G, FIG. 1F, Kruskal-Wallis test, p<0.0001, FIG. 1G, One-way ANOVA, p<0.0001), which is in line with the robust increase in the number of synapses observed (FIG. 1B-1D). hUTC also increased the amplitude of potsynaptic currents. Like astrocytes, hUTC strengthen synaptic activity (FIGS. 1H,1I Kruskal-Wallis test, p<0.0001). Waveform of mEPSC peaks revealed that both rising and decay tau are increased with hUTC treatment (FIGS. 1J-1M). hUTC induce excitatory synapse formation and enhance synaptic function in cultured RGCs, similar to ASCs.

### Example 2

### Effect of Conditioned Media on Neurite Outgrowth and Synaptogenesis

The hUTC-conditioned media was assessed for effects in culture with RGCs.

hUTC, RGCs and astrocytes were obtained as described above in Example 1.

Preparation of Conditioned Media:

Pure astrocyte cultures or hUTC were grown in 10-cm tissue culture dishes (Corning, Corning, N.Y.) in their own growth media until they were 70% confluent. Cells were then washed twice with warm DPBS (Gibco) and 10 mL of conditioning media, which is composed of Neurobasal® (Gibco) supplemented with L-glutamine (2 mM, Gibco), sodium pyruvate (1 mM, Gibco), penicillin (100 U/mL) and streptomycin (100 μg/mL, Gibco), was added to each dish. Media were conditioned by cells for 5 days. Cell-free conditioned media were collected and concentrated 10 times by using SkDa molecular weight cut-off Vivaspin 20 centrifugal concentrators (Sartorius, Bohemia, N.Y.). The protein concentration of each conditioned media was measured by the Bradford assay (Thermo Scientific, Grand Island, N.Y.) following the manufacturer's recommendations. Aliquots of Astrocyte-Conditioned-Media (ACM) and hUTC-Conditioned Media (UCM) in low protein-binding tubes (Eppendorf, Hamburg, Germany) were rapidly frozen in liquid nitrogen, and were stored at −80° C. until use. To fractionate UCM with different molecular weight cut-offs, 30 kDa and 100 kDa Vivaspin 20 centrifugal concentrators were used. To collect 5-100 kDa UCM fractions, flow-through of 100 kDa Vivaspin 20 was concentrated with 5 kDa Vivaspin 20 concentrators. To collect 5-100 kDa UCM fractions, flow-through of 100 kDa Vivaspin 20 was concentrated with 5 kDa Vivispan concentrators. For treatments, frozen aliquots were allowed to slowly melt on ice, and conditioned media were added into ice-cold RGC growth media (synapse and outgrowth assays) or minimal media (survival assays) at the desired concentrations.

Western Blot Analysis of Conditioned Media:

10-20 μg of conditioned media were prepared for poly-acryl-amide gel electrophoresis (PAGE) in 5× Laemmli loading buffer (Thermo Scientific) containing 5% (3-mercaptoethanol (v/v). The proteins were denatured at 95° C. for 15 minutes. Proteins were separated in 10% SDS-PAGE gels (BioRad, Hercules, Calif.) and were then transferred onto PVDF membranes (Millipore). To detect TSPs; goat anti-human TSP1 (1:250, RRID: AB_2201958), TSP2 (1:250, RRID: AB_220268), TSP4 (1:200, RRID: AB_2202087) (R&D Systems) were used. For loading control another hUTC-secreted protein, called Hevin/SPARCL1, was detected using a goat anti-Hevin antibody (1:250, RRID: AB_2195103, R&D systems). Horseradish peroxidase (HRP) conjugated anti-goat antibody (R&D, 1:5000) was used as secondary antibody. The detection was performed with an Amersham™ ECL Western Blotting Analysis System kit (GE Healthcare, Winterville, N.C.) or SuperSignal® West Femto Maximum Sensitivity Substrate (Thermo Scientific) following the manufacturer's recommendation.

Synapse assay, electrophysiological recordings, and neurite outgrowth assay are described in Example 1.

Cell Survival Assay:

RGCs were plated directly onto 24-well tissue culture plates (7,500 cells/well). Wells were coated with PDL and laminin. Cells were cultured in a minimal media containing Neurobasal® (Invitrogen), SATO supplement (100 μg/ml Transferrin, 100 μg/ml bovine serum albumin, 60 ng/ml progesterone, 16 μg/ml putrescine, 40 ng/ml sodium selenite), N-acetyl cysteine (5 μg/ml), triiodo-thyronine (4 μg/ml), L-glutamine (2 mM), penicillin (100 U/mL), streptomycin (100 μg/mL), sodium pyruvate (1 mM) and forskolin (5 μM). To test the effects of UCM and ACM on survival, the minimal media were supplemented with various concentrations of conditioned media in the absence/presence of forskolin, BDNF (200 ng/mL) or CNTF (40 ng/mL). The cell viability was assessed at 3 DIV by using the LIVE/DEAD® Viability Kit for mammalian cells (Invitrogen) following the manufacturer's instructions. The viability counts were performed 15 minutes after the application of the kit agents and representative images were captured with Zeiss Axio Observer A1 Epifluorescence Microscope (Carl Zeiss) using a 20× objective. At least 10 fields per treatment were counted in each experiment and the results presented are an average of 2-3 experiments. Percent survival was calculated as the number of live cells divided by the total of number of dead and live cells times 100.

Statistical analyses of the quantified data were performed as described in Example 1.

### Results

RGCs were fed with various concentrations of hUTC-conditioned medium (hUCM). hUTC were cultured alone and hUTC-conditioned media (UCM) collected (FIG. 2A). RGCs were treated with UCM and assessed for the effect of conditioned media on synapse number and function using the same assays as in Example 1. Synapse analysis showed that hUCM was sufficient to induce synapse formation of RGCs and enhance synaptic function similar to the hUTC in transwell inserts (FIGS. 2B-2N) in a concentration dependent manner, similar to astrocyte-conditioned media (ACM). (FIGS. 2B-2D and 2J). UCM showed full synaptogenic activity at concentrations between 20-80 μg total protein/mL culture media. UCM may be substituted for hUTC co-culture. hUCM also strengthened functional synapses as shown by increased amplitude and frequency of mEPSCs. (FIGS. 2E-2I).

These results demonstrate that hUTC induced synapse formation and enhance synaptic function. The synaptogenic effect of hUTC contributes to the functional recovery that occurs by the delivery of these cells to animal disease models.

Besides its effects on synapse formation, hUCM promoted RGC survival in the absence of any other growth factors, for example, BDNF, and CTNF. (FIGS. 3A, 3D and 3E). RGCs were cultured for 3 DIV in a minimal media, which lacked the media supplement B27 and growth factors BDNF, CNTF and insulin, but contained forskolin (referred as CTR for minimal media control condition). Under these minimal media conditions less than 5% of the RGCs survive at the end of 3 DIV (FIG. 3A, left panel and FIG. 3B). Addition of growth factors such as BDNF or CNTF, only in the presence of forskolin, increased RGC survival (Meyer-Franke A, et al., Neuron., 1995; 15:805-819). Forskolin in the culture media increases cAMP levels in neurons mimicking ongoing neuronal activity, which is critical for survival of CNS neurons (Meyer-Franke et al., 1995). The addition of UCM into minimal media stimulated RGC survival in a concentration-dependent manner (FIG. 3A, right panel and FIG. 3B); whereas ACM did not promote RGC survival at the same concentrations (FIG. 3B).

The survival-promoting activity of UCM was functional in the presence of forskolin in the minimal medium (FIG. 3C). These results demonstrate that UCM stimulated RGC survival. At lower concentrations (40 μg/ml), the survival effect of UCM appears additive to that of BDNF or CNTF (FIG. 3D, 3E).

UCM also enhanced RGC neurite outgrowth as demonstrated by an increase in total process length, number of processes and number of branches. (FIGS. 3F-3J). RGC growth media with UCM at the time of plating showed that UCM contains factors that promote neurite outgrowth and elaboration (FIGS. 3F-3J). ACM also promoted neurite outgrowth (FIGS. 3F-3J); UCM in general was more efficient in inducing overall elaboration and branching (FIGS. 3F and 3J). Sholl analysis showed that UCM increases neuronal complexity, when compared to RGCs cultured alone (FIG. 3G).

In summary, hUTC secrete factors that promote development of functional synapses between purified RGCs in vitro. Moreoever, hUTC also support neuronal survival and growth.

### Example 3

### Secretion of Synaptogenic Factors by Progenitor Cells

It has been shown that the secretome from hUTC positively effects development of functional synapses, and neuronal survival and outgrowth. In this example, the synaptogenic factors secreted by hUTC are identified.

Immunocytochemistry Assay:

hUTC and RGCs were obtained as described above in Example 1. hUTC conditioned media (UCM) was prepared as in Example 2.

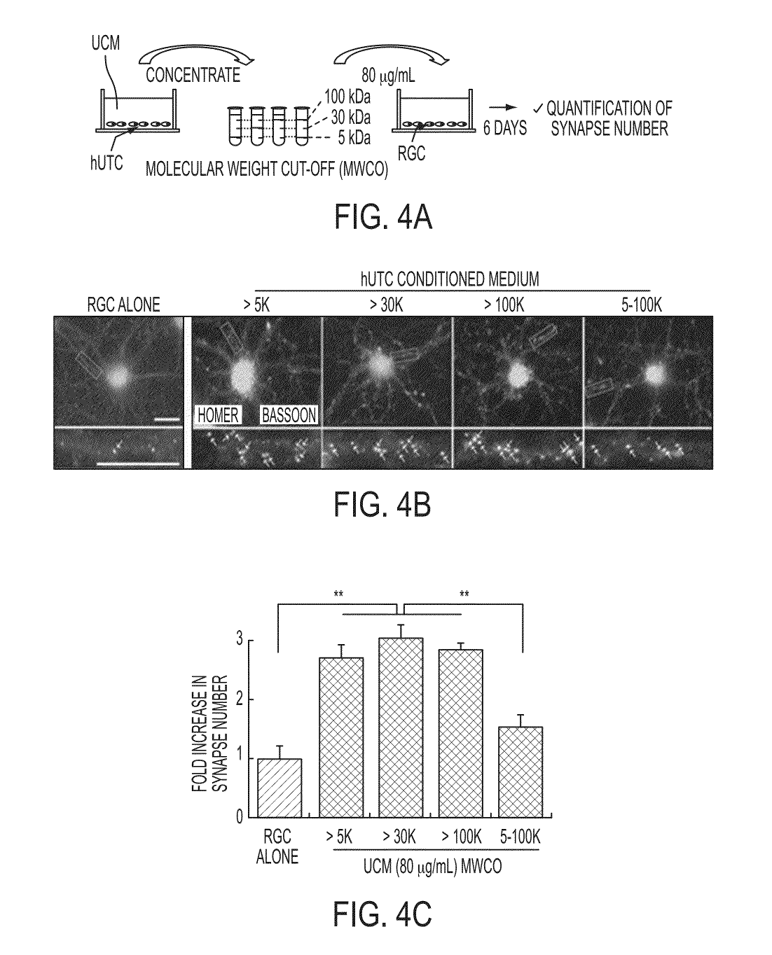

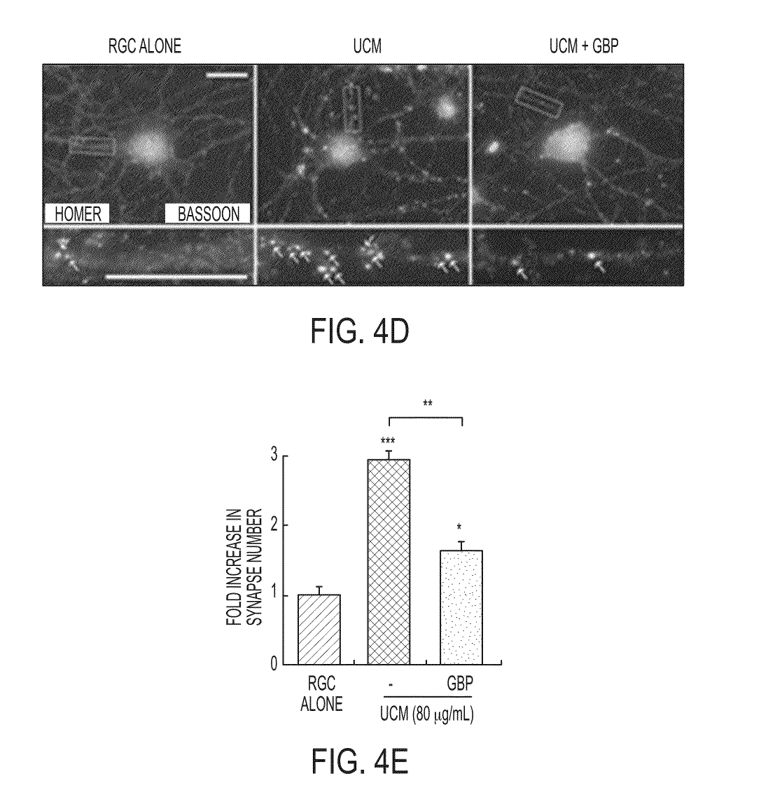

Proteins in UCM were separated by Molecular Weight Cut-Off (MWCO) within about 5 kDa to about 100 kDa. 80 ug/mL UCM was used for culturing RGCs and analyzed by immunocytochemistry synapse assay. Isolated proteins >5 kDa, >30 kDA, and >100 kDa were compared to RGC alone and UCM 5-100 kDa. (FIG. 4A).

The synaptogenic blocker Gabapentin (GBP) was used for further identification of synaptogenic factors secreted by hUTC.

Neurite Outgrowth Assay:

To test neurite outgrowth under pathologic (i.e. growth-inhibiting) culture conditions, the RGCs (1,500 cells/coverslip) were seeded onto coverslips that were coated with various concentrations of chondroitin sulfate proteoglycan (CSPG, EMD Millipore, Billerica, Mass.), Nogo-A (R&D Systems, Minneapolis, Minn.) or in the presence of soluble myelin basic protein (MBP, 10 ug/mL, Sigma-Aldrich, St. Louis, Mo.). After 24 hours, neurites were visualized by incubating with CellTracker Red CMPTX dye (Inivtirogen) for 15 mins followed by fixation with 4% PFA (w/v). Images of single RGCs with their neurites were captured using a 20× objective on a Zeiss Axioimager M1 Epifluorescence Microscope (Carl Zeiss). At least 30 cells per condition were imaged and analyzed per experiment and the results presented are the average of 2-3 independent experiments. Neuronal morphology was analyzed using neurite outgrowth application module in Metamorph software (Molecular Devices). Sholl analysis was performed using a plug-in for Fiji as described in Ferreira T A, et al., Nature Methods, 2014; 11:982-984.

Knockdown Assay:

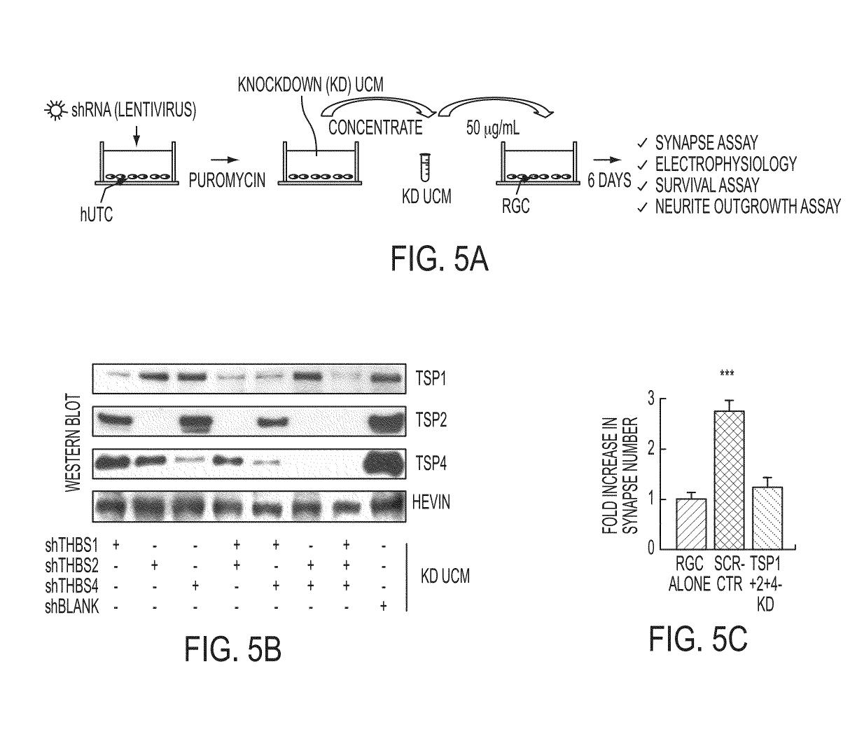

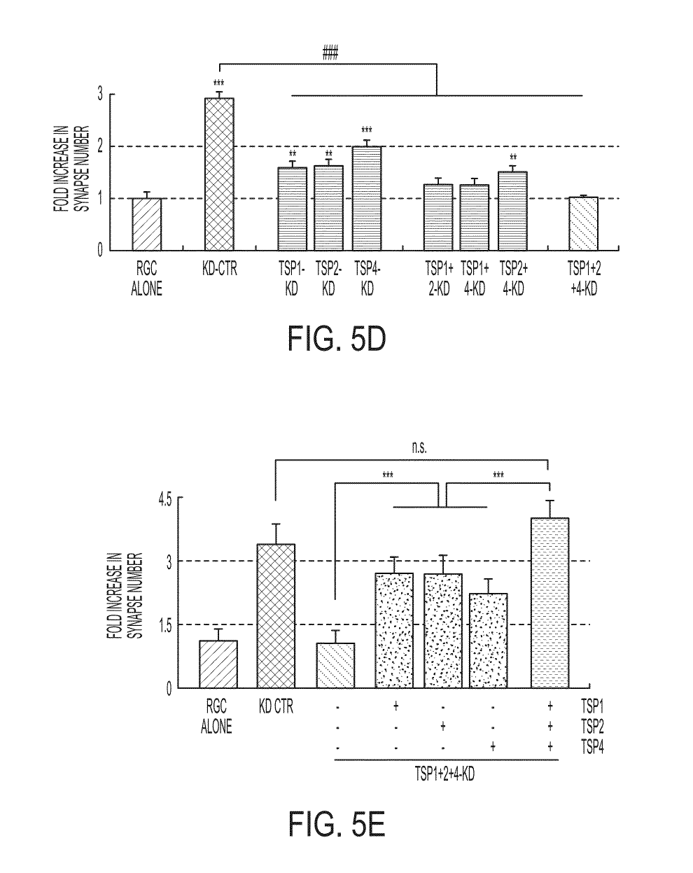

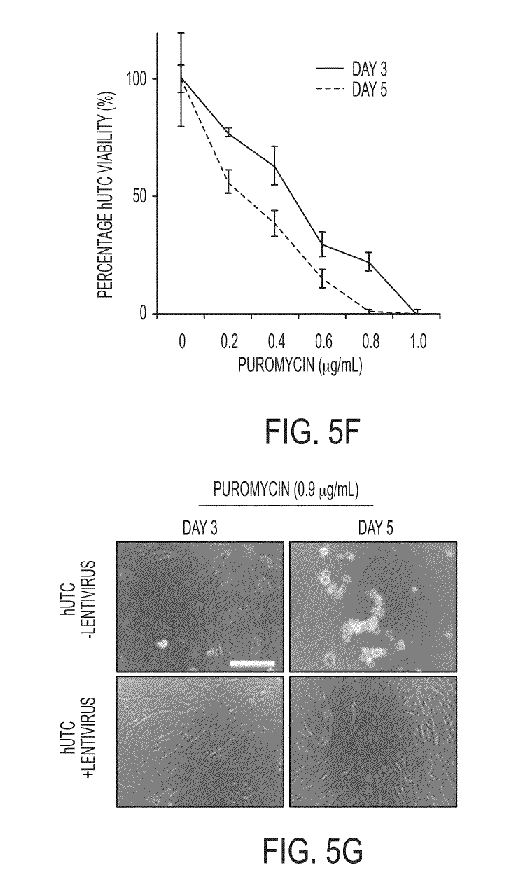

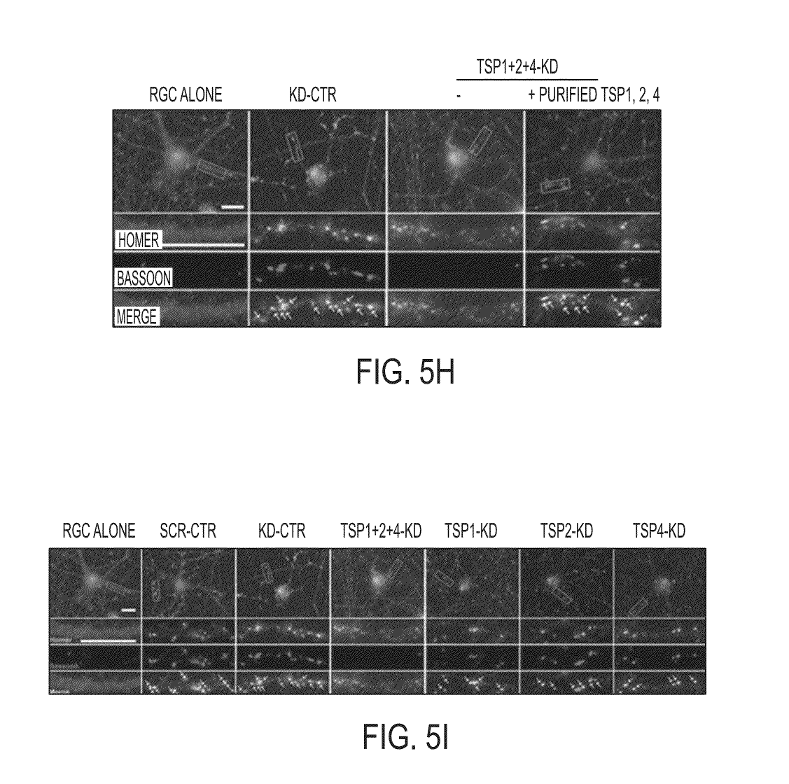

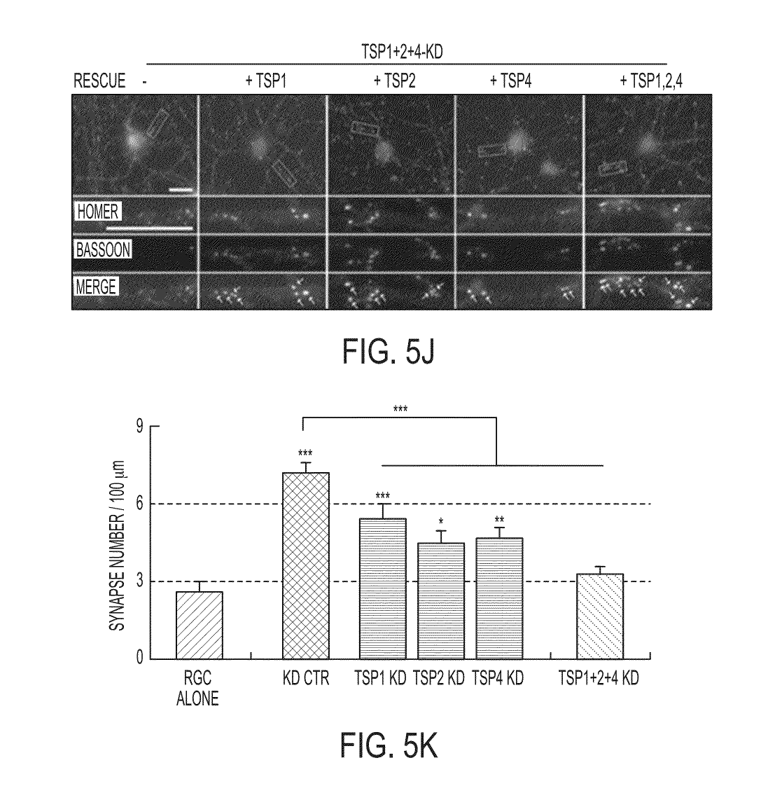

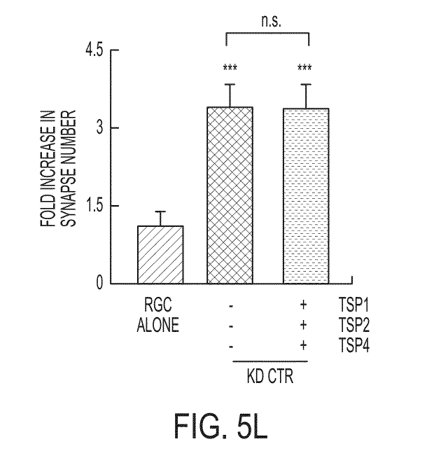

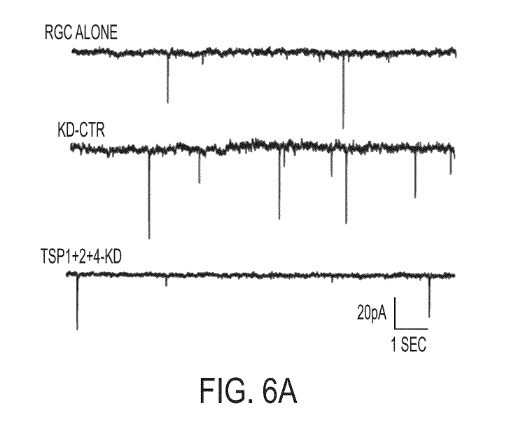

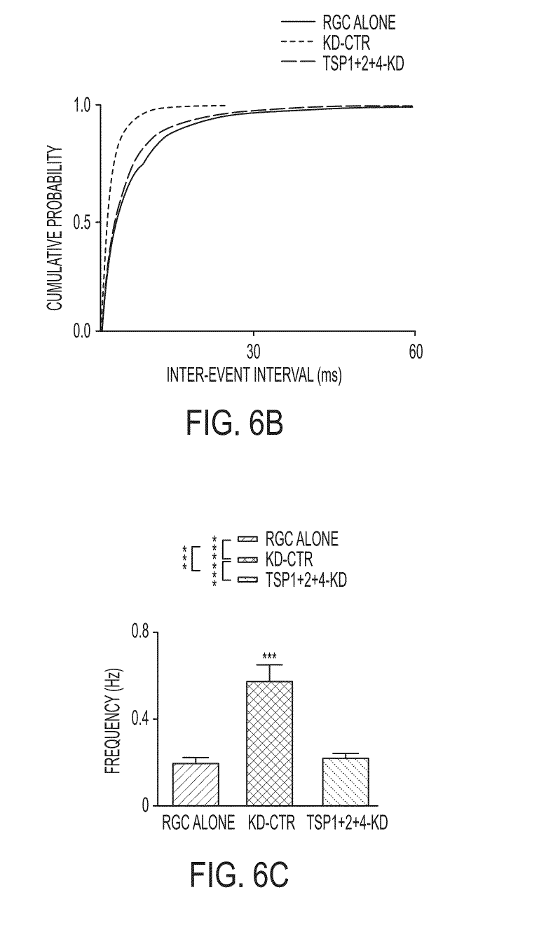

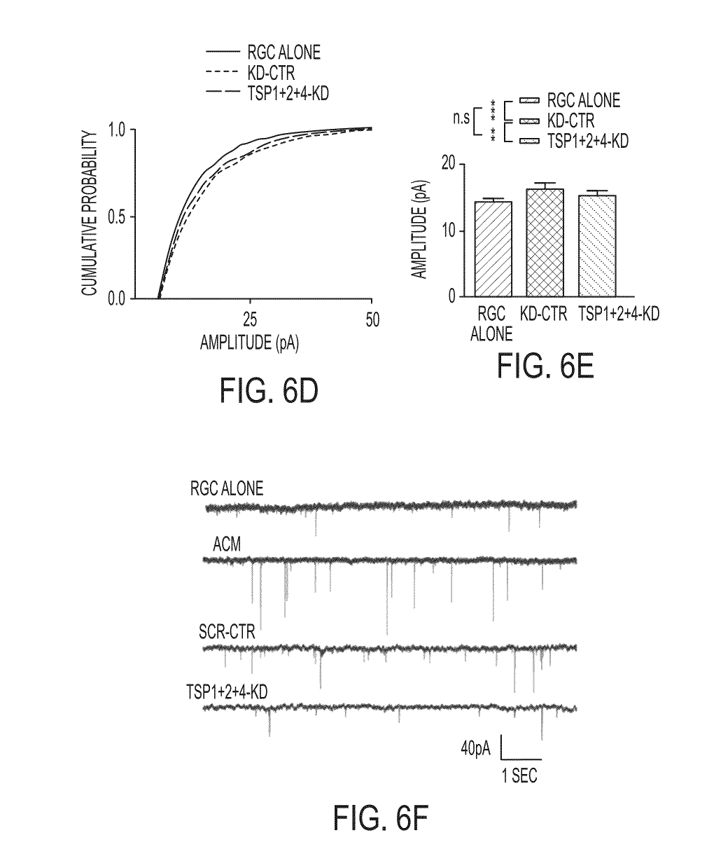

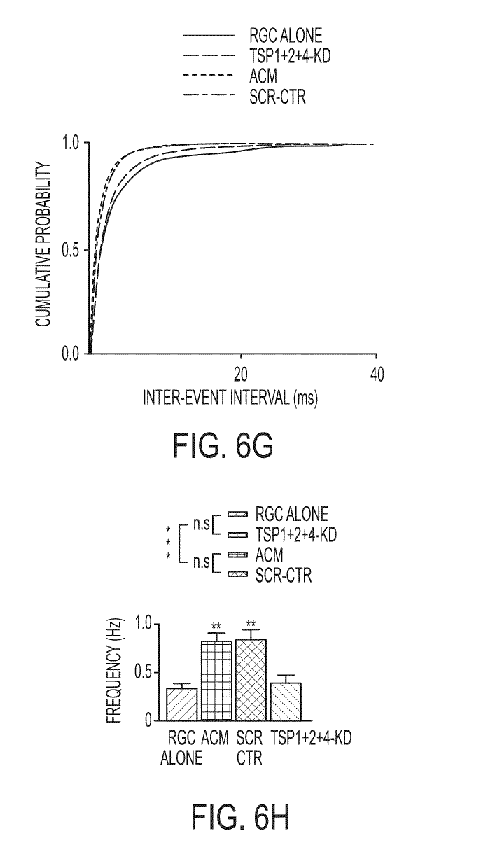

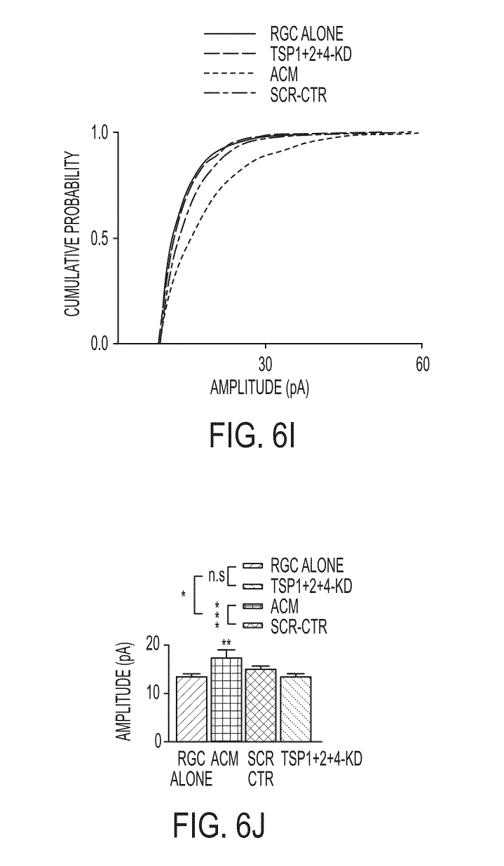

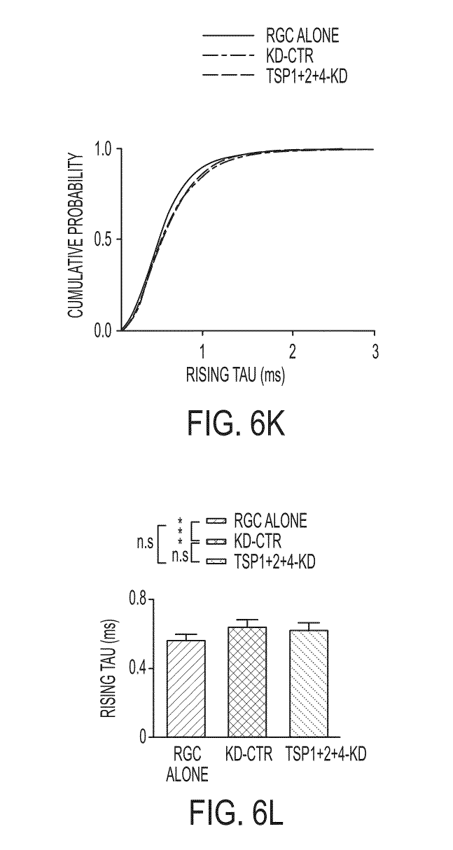

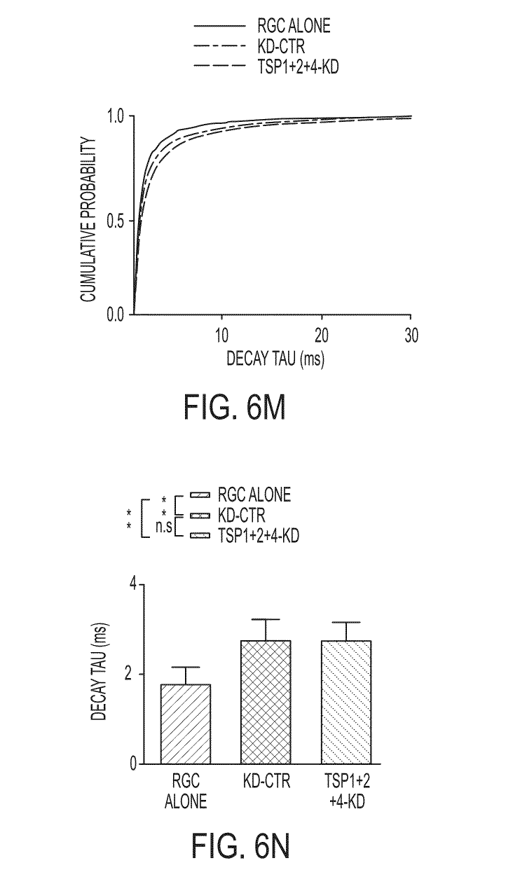

hUTCs were cultured with Lentivirus shRNA to prepare knockdown (KD) UCM. RGCs were then cultured in the KD UCM and assayed for synapse formation, electrophysiology, and neuronal survival and outgrowth. (FIG. 5A). shRNA construct pools cloned into pLKO.1-puro vectors that target human THBS1, THBS2 or THBS4 mRNA (that are translated to TSP1, TSP2 and TSP4, respectively) were purchased from Thermo Scientific. The empty pLKO.1 puro vector was purchased from Addgene (plasmid 8453, Cambridge, Mass.) and used as knockdown control. Knockdown efficiency of individual constructs was determined by transfection into hUTC. An shRNA construct for each TSP that demonstrated the most effective knockdown was chosen for subsequent lentivirus production.

To make the lentivirus, 293T lentiviral-packaging cells were seeded at 8×10⁶ cells per T75 flask a day prior to transfection. The next day, pLKO.1-puro lentiviral plasmid containing shRNA for Thbs1 (clone number TRCNO0224), Thbs2 (clone number TRCN53972), Thbs4 (clone number TRCN54048), or knockdown control (Addgene plasmid 8453) was transfected into 293T cells. For scrambled shRNA controls, oligos containing the same sense and antisense scrambled sequences for Thbs1 (5′-ATAACTCCGATCGTTCAATAT-3′), Thbs2 (5′-GTTACATCTCGATACGATACA-3′) and Thbs4 (5′-ATATAAGACGCTAGATCCACA-3′) shRNAs were used. The scrambled shRNA oligos were annealed and cloned into pLKO.1-puro and used to produce lentivirus. Packaging plasmids, delta R8.2 and VSV-G were co-transfected using X-tremeGENE transfection reagent (Roche, Basel, Switzerland) following the manufacturer's recommendation. Lentiviral supernatants were collected twice at 48 and 72 hours post-transfection, and filtered through a 0.45 μm filter (Millipore). The viral particles were detected using Lenti-X™ GoStix™ (Clontech, Mountain View, Calif.). The filtered supernatants were used to infect hUTC after supplementation with polybrene (2 μg/ml, Sigma). The transduced hUTC were further selected by adding puromycin (900 ng/mL) to the hUTC media for 5 days before conditioning media for the RGC experiments.

Production and Purification of Recombinant TSPs:

Purified recombinant human TSP1 was purchased from R&D Systems. For TSP2 purification, a CHO cell line-expressing mouse TSP2 was used to produce conditioned media. The secreted recombinant TSP2 was purified from the culture media as previously described (Oganesian A, et al., Molecular Biology of Cell, 2008; 19:563-571) using affinity chromatography procedures with HiTRAP heparin HP (GE Healthcare). For TSP4, a 6-Histidine tagged rat TSP4 construct (pcDNA3-TSP4, as described in Kim D S, et al., J Neurosci, 2012; 32:8977-8987) was transfected into HEK293 cells using Lipofectamine 2000 (Invitrogen) following manufacturer's instruction. The secreted recombinant TSP4 was purified from the culture media by Ni-chelating chromatography using Ni-NTA resin (Qiagen, Venlo, Netherlands) following the manufacturer's instructions. Purified recombinant TSPs were concentrated up to 1 mg/mL with 30 kDa cut off Vivaspin 20 (Sartorius) and aliquots were rapidly frozen in liquid nitrogen and were stored at −80° C. until use.

Synapse assay, electrophysiological recordings, and neurite outgrowth assay are described in Example 1. Statistical analyses of the quantified data were performed as described in Example 1. Western blot analysis of conditioned media is described in Example 2.

### Results

Synaptogenic factors secreted by hUTC fractionated UCM using different molecular weight cut-off size exclusion columns (FIG. 4A) provides that the synaptogenic effect of UCM concentrates within the fraction that is larger than 100 kDa (FIGS. 4B, 4C). Immunocytochemistry of UCM culture with RGCs showed synaptogenic factors in the 5-100 kDa range were minimal, with the highest effect from factors larger than 100 kDa. (FIGS. 4B, 4C). In agreement with a role for TSPs in UCM-induced synapse formation, addition of GBP blocked the synaptogenic factors. (FIGS. 4D, 4E). Gabapentin is a known blocker of synaptogenic thrombospondin family, indicating that TSP family proteins are the major synaptogenic factors secreted by hUTC.

Isoform specific shRNAs were used to knock-down TSP1, 2 and 4 individually or in combination using a lentivirus-mediated approach. (FIG. 5A), confirming TSPs as synaptogenic factors in hUTC CM. Specific knockdown of each TSP by the corresponding shRNA was confirmed by Western blotting (FIG. 5B). UCM produced from the lentiviral transduction of same parent plasmid vector without shRNAs (KD-CTR) or triple infected with lentivirus containing scrambled TSP1, 2 and 4 shRNAs (SCR-CTR) were used as controls. UCM were produced from lentivirus-infected hUTC and applied to RGCs to identify the effects of TSP knockdown (FIG. 5A). shRNA constructs for TSP-1, TSP-2 and TSP-4 delivered to hUTC resulted in knockdown of expression for each TSP. (FIGS. 5B and 5D). Quantification of synapse numbers and synapse density in RGCs revealed that all three TSPs, (TSP1, TSP2 and TSP4) contributed to the synaptogenic function of UCM (FIGS. 5C, 5D, 5H-5J). Individual knockdown of TSPs decreased the number of synapses compared to KD-CTR UCM (FIGS. 5C, 5D, 5H-5J). Knocking-down all three TSPs (TSP1+2+4-KD) abolished the synaptogenic effect of UCM (FIGS. 5C, 5D, 5J).

Supplementing the TSP1+2+4-KD UCM with pure TSP1, TSP2 and TSP4, (150 ng/ml each) restored the synaptogenic effect of the TSP1+2+4-KD UCM (FIGS. 5C, 5E, 5I). Adding pure TSPs into KD-CTR UCM did not further increase the synapse numbers (FIG. 5K).

mEPSCs electrophysiology recordings show that silencing expression of the TSPs reduced the synaptogenic effects of the hUTC, with a decreased amplitude and frequency of mEPSCs. RGCs that were treated with ACM (positive control), SCR-CTR or TSP1+2+4-KD UCM showed that silencing of TSP expression diminished the UCM-induced increase in the frequency of synaptic events (FIG. 6F, 6G, 6H). This is in agreement with findings that TSPs, 1, 2 and 4 are integral for UCM to induce synaptogenesis (FIG. 5). TSP knockdown had milder effects on mEPSC peak properties such as the amplitude (FIG. 6I, 6J) and rising tau (FIGS. 6K, 6L). Moreover, TSP knockdown did not affect the ability of UCM to increase decay tau (FIGS. 6M, 6N). Silencing of TSPs 1, 2, and 4 in hUTC eliminates the ability of these cells to induce an increase in synapse numbers. Loss of TSPs does not completely diminish the effects of UCM on the amplitude of synaptic currents and the waveform of mEPSC peaks.

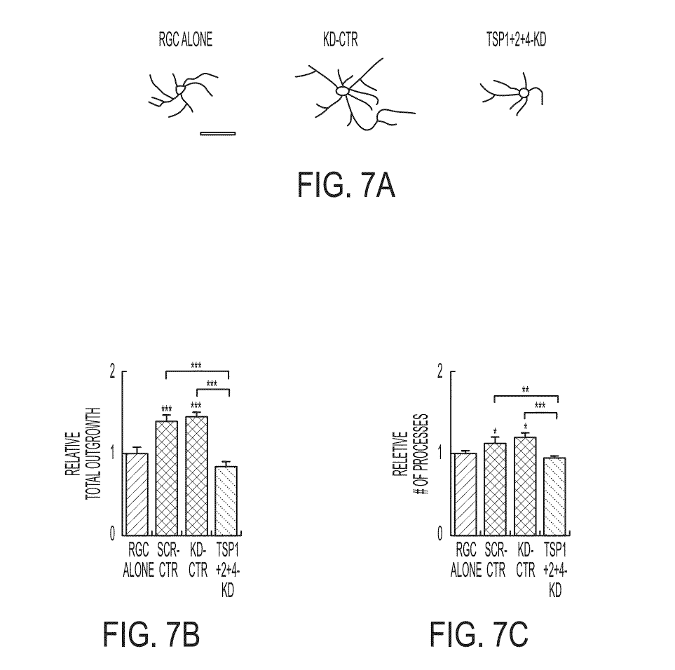

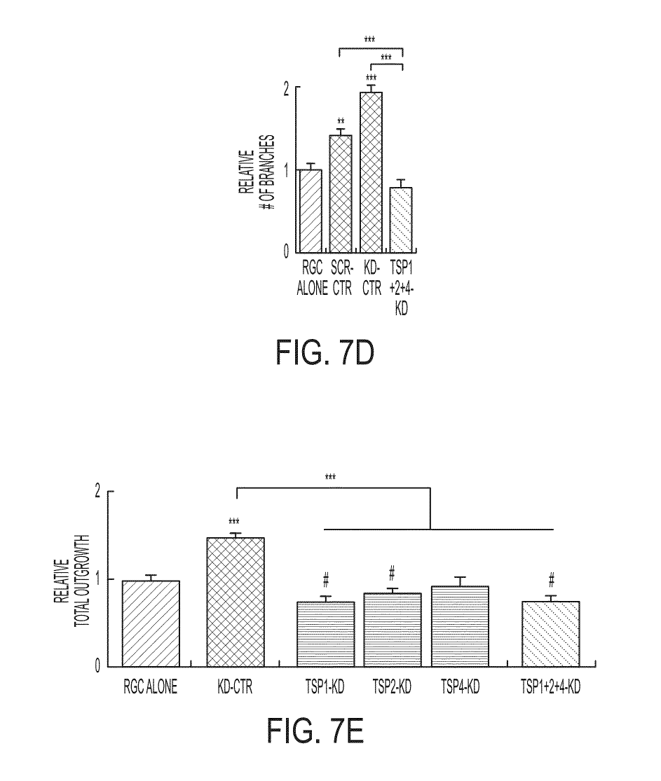

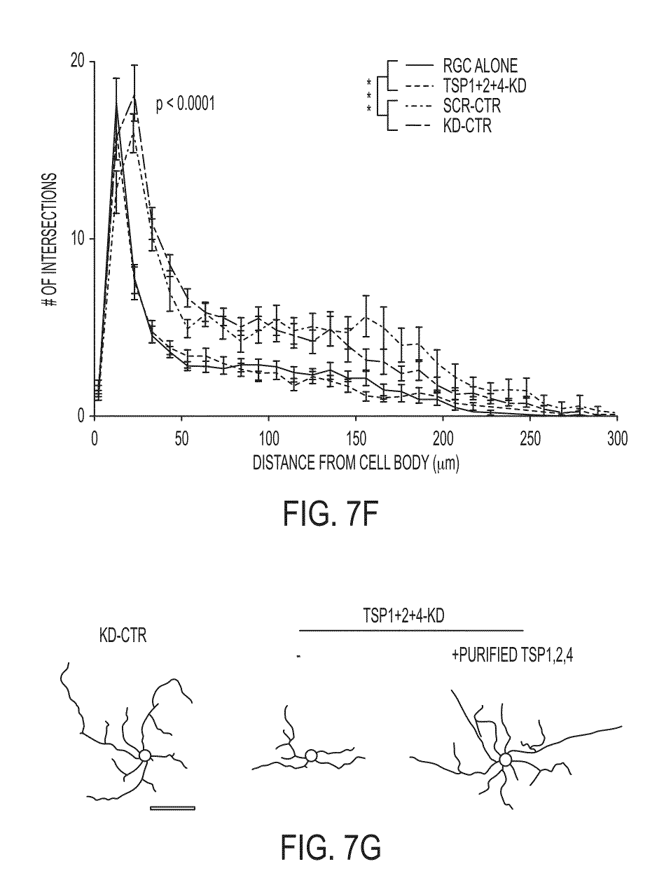

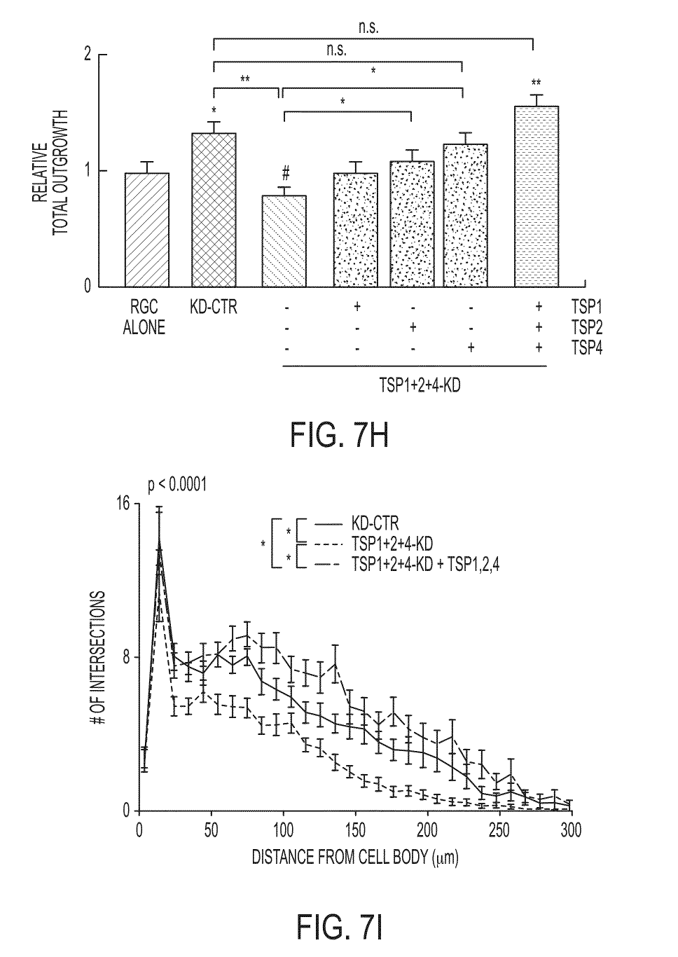

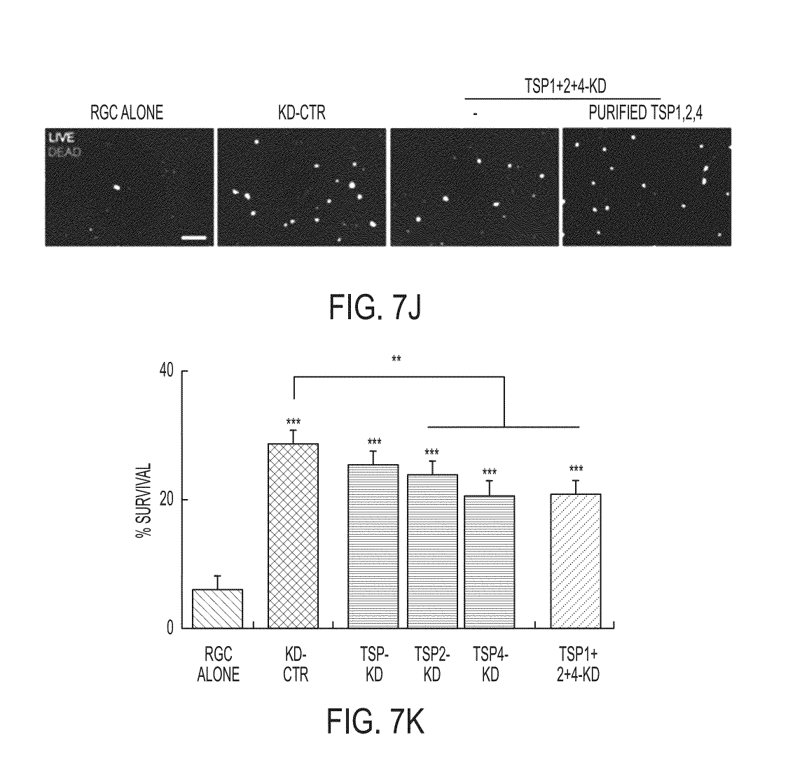

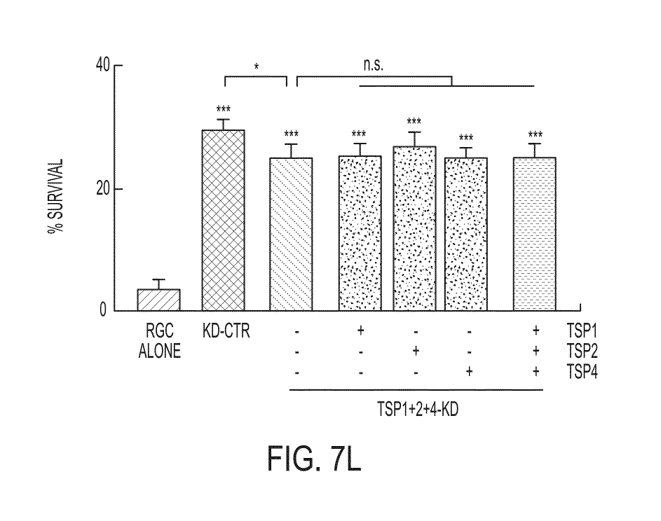

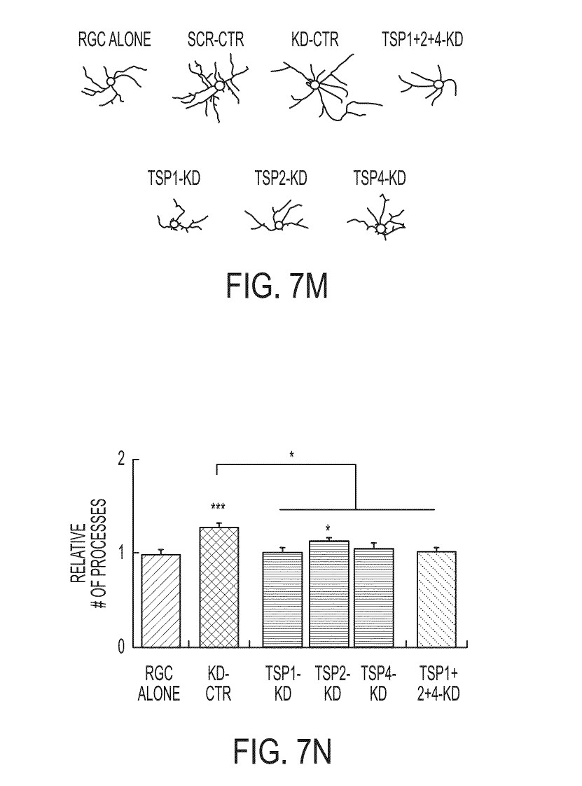

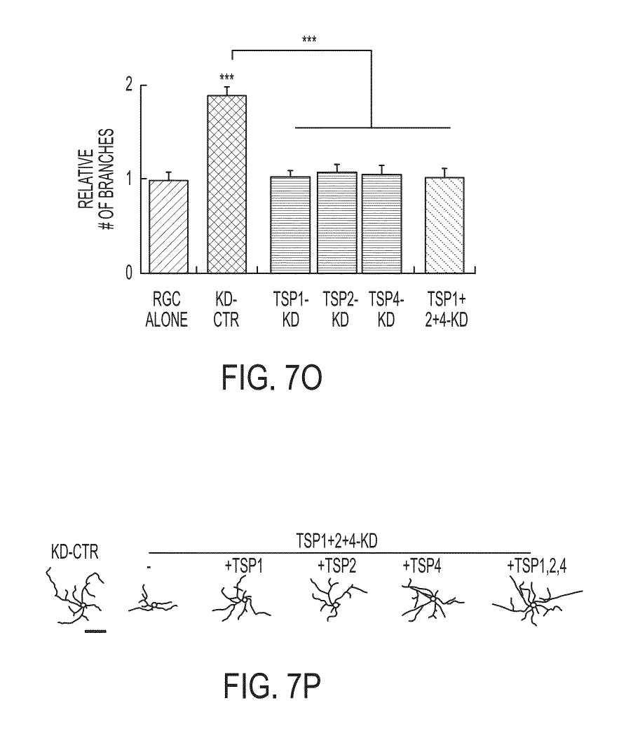

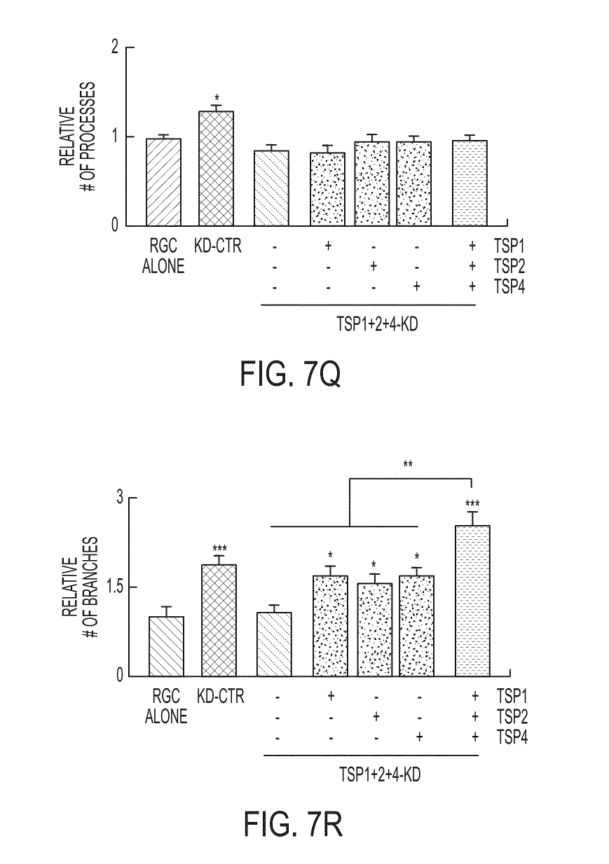

In addition, silencing TSP expression abolished neurite outgrowth effects of UCM (FIGS. 7A, 7B, 7C, 7D, 7E). Single knockdowns of TSP1, 2 or 4 abolished neurite outgrowth-promoting effects of UCM (FIG. 7E), including the effects on the number of processes and the number of branches (FIGS. 7N-7O). Knockdown of TSP2 or TSP4 or all three TSPs, but not TSP1 alone, decreased the survival-promoting activity of the UCM (FIG. 7K). Supplementing the TSP1+2+4-KD UCM with pure TSPs either individually or all together did not rescue the survival-promoting function of the UCM (FIGS. 7L). Supplementing the TSP1+2+4-KD UCM with pure TSPs (TSP1, TSP2 and TSP4) (150 ng/ml each) recovered neurite outgrowth-stimulating function of the UCM (FIGS. 7H, 7I, 7Q, 7R).

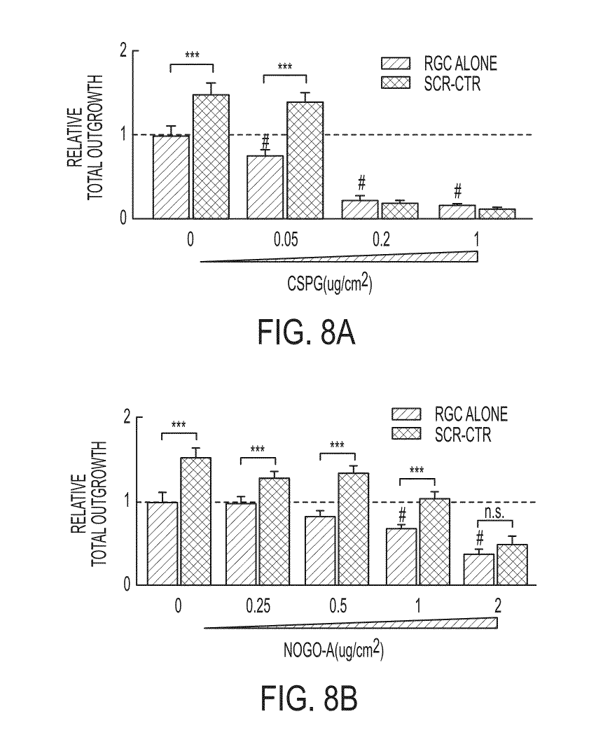

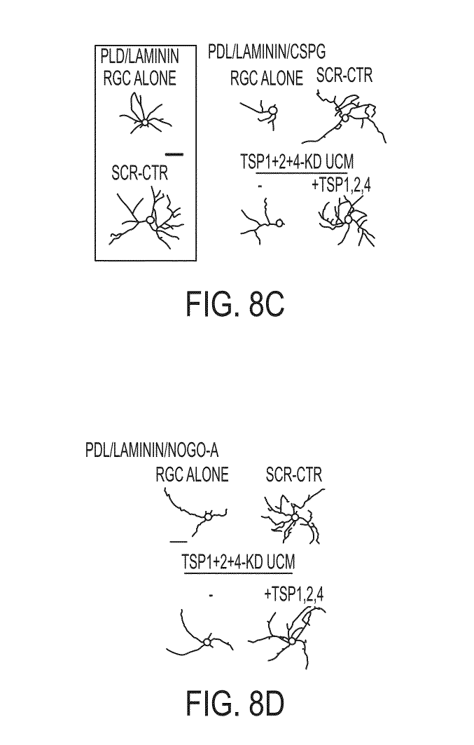

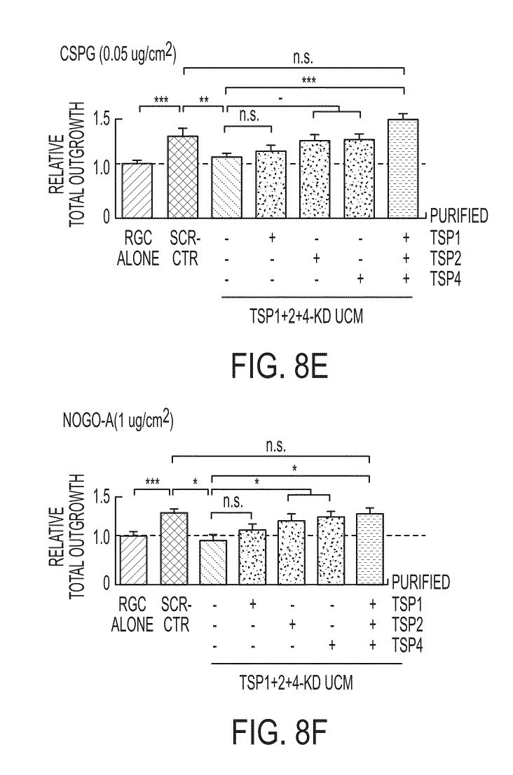

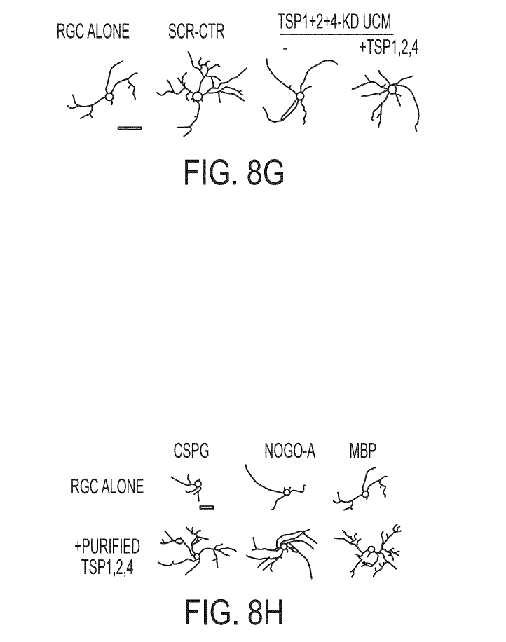

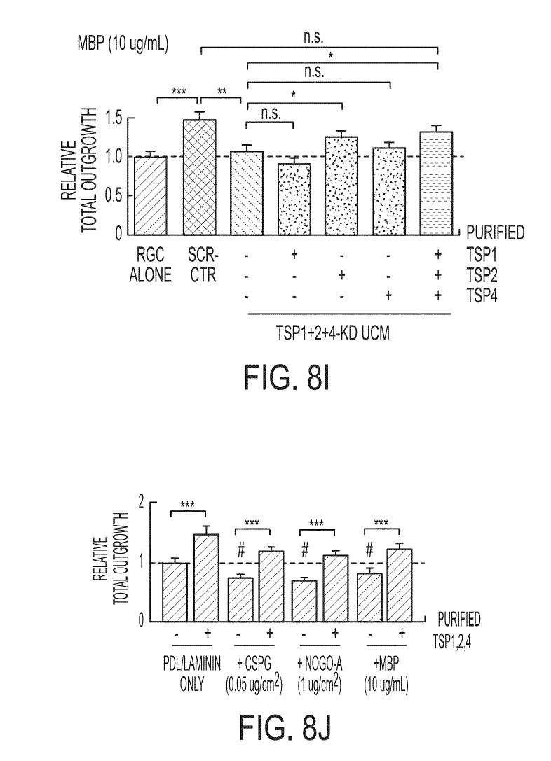

Upon CNS injury, chondroitin sulfate proteoglycans (CSPGs) produced by reactive glia, and myelin proteins, such as Nogo A, are released from degenerating neurons. These proteins inhibit axon regeneration (Niederost et al., 2002; Silver and Miller, 2004; Usher et al., 2010; Walker et al., 2012), impeding neuronal repair and injury. Modified RGC neurite outgrowth assay illustrates hUTC promote neurite outgrowth under conditions that inhibit growth. In the modified RGC neurite outgrowth assay using CSPG or Nogo-A (FIGS. 8A and 8B), CSPG hindered neurite outgrowth starting at 0.05 μg/cm² and completely blocked growth at 0.2 μg/cm² or higher concentrations (FIG. 8A). Nogo-A blocked neurite outgrowth in RGCs starting at 1 μg/cm² with more than 50% of neurite growth inhibited at 2 μg/cm² (FIG. 8B). UCM transduced with lenti-viruses that encode scrambled shRNAs (SCR-CTR UCM) induced neurite outgrowth in RGCs that were plated onto coverslips with 0.05 μg/cm² CSPG or 1 μg/cm² Nogo-A (FIGS. 8A and 8B). hUCM induced neurite outgrowth even under growth-inhibiting conditions. SCR-CTR UCM was not able to trigger neurite outgrowth at high CSPG (>0.2 μg/cm²) and Nogo-A (2 μg/cm²) concentrations (FIGS. 8A and 8B).

Unlike SCR-CTR UCM, TSP1+2+4-KD UCM did not trigger neurite outgrowth when 0.05 μg/cm² CSPG (FIGS. 8C and 8E) or 1 μg/cm² Nogo-A (FIGS. 8D and 8F) were present, indicating that TSPs are important for the neurite outgrowth-promoting effects of hUCM under growth-inhibiting conditions. Addition of purified TSP2 or TSP4 alone or all three TSPs together rescued the ability of TSP1+2+4-KD UCM to induce neurite outgrowth in the presence of CSPG or Nogo-A (FIGS. 8C-8F). These results show that TSPs, particularly TSP2 and 4, are responsible for the growth-stimulating effects of hUCM in the presence of CSPG or Nogo-A.

Myelin debris that is generated by CNS injury also sheds high levels of the myelin protein MBP (Liu et al., 2006; Stapulionis et al., 2008). RGC outgrowth assay in the presence of 10 μg/ml soluble MBP showed MBP had neurite-outgrowth inhibiting effects on CNS neurons. Previous study showed that MBP induces neurotoxicity at concentrations higher that 10 μg/ml (Zhang et al., 2014). Here, MBP at 10 μg/ml leads to loss of neurite outgrowth in RGCs (FIG. 8J, 14±0.07% reduction in outgrowth compared to RGCs culture under normal growth media conditions). SCR-CTR UCM induced neurite outgrowth in the presence of MBP and this effect of the UCM was lost when TSPs were silenced (i.e. TSP1+2+4-KD UCM, FIGS. 8G and 8I). Only the addition of pure TSP2 back into the TSP1+2+4-KD UCM rescued the outgrowth promoting effect. These results show that in the presence of MBP, TSP2 can mediate neurite outgrowth, and under growth inhibiting conditions, hUTC-secreted TSPs, TSP2 and TSP4, mediate neurite outgrowth. The addition of pure TSPs to the growth media also induced RGC neurite outgrowth in the presence of CSPG, Nogo-A or MBP (FIGS. 8H and 8J).

### Example 4

### Derivation of Cells from Postpartum Tissue

This example describes the preparation of postpartum-derived cells from placental and umbilical cord tissues. Postpartum umbilical cords and placentae were obtained upon birth of either a full term or pre-term pregnancy. Cells were harvested from five separate donors of umbilicus and placental tissue. Different methods of cell isolation were tested for their ability to yield cells with: 1) the potential to differentiate into cells with different phenotypes, a characteristic common to stem cells; or 2) the potential to provide trophic factors useful for other cells and tissues.

### Methods & Materials

Umbilical Cell Isolation:

Umbilical cords were obtained from National Disease Research Interchange (NDR1, Philadelphia, Pa.). The tissues were obtained following normal deliveries. The cell isolation protocol was performed aseptically in a laminar flow hood. To remove blood and debris, the cord was washed in phosphate buffered saline (PBS; Invitrogen, Carlsbad, Calif.) in the presence of antimycotic and antibiotic (100 units/milliliter penicillin, 100 micrograms/milliliter streptomycin, 0.25 micrograms/milliliter amphotericin B). The tissues were then mechanically dissociated in 150 cm² tissue culture plates in the presence of 50 milliliters of medium (DMEM-Low glucose or DMEM-High glucose; Invitrogen), until the tissue was minced into a fine pulp. The chopped tissues were transferred to 50 milliliter conical tubes (approximately 5 grams of tissue per tube).

The tissue was then digested in either DMEM-Low glucose medium or DMEM-High glucose medium, each containing antimycotic and antibiotic as described above. In some experiments, an enzyme mixture of collagenase and dispase was used (“C:D”) collagenase (Sigma, St Louis, Mo.), 500 Units/milliliter; and dispase (Invitrogen), 50 Units/milliliter in DMEM-Low glucose medium). In other experiments a mixture of collagenase, dispase and hyaluronidase (“C:D:H”) was used (collagenase, 500 Units/milliliter; dispase, 50 Units/milliliter; and hyaluronidase (Sigma), 5 Units/milliliter, in DMEM-Low glucose). The conical tubes containing the tissue, medium and digestion enzymes were incubated at 37° C. in an orbital shaker (Environ, Brooklyn, N.Y.) at 225 rpm for 2 hrs.

After digestion, the tissues were centrifuged at 150×g for 5 minutes, and the supernatant was aspirated. The pellet was resuspended in 20 milliliters of Growth Medium (DMEM-Low glucose (Invitrogen), 15 percent (v/v) fetal bovine serum (FBS; defined bovine serum; Lot#AND18475; Hyclone, Logan, Utah), 0.001% (v/v) 2-mercaptoethanol (Sigma), 1 milliliter per 100 milliliters of antibiotic/antimycotic as described above. The cell suspension was filtered through a 70-micrometer nylon cell strainer (BD Biosciences). An additional 5 milliliters rinse comprising Growth Medium was passed through the strainer. The cell suspension was then passed through a 40-micrometer nylon cell strainer (BD Biosciences) and chased with a rinse of an additional 5 milliliters of Growth Medium.

The filtrate was resuspended in Growth Medium (total volume 50 milliliters) and centrifuged at 150×g for 5 minutes. The supernatant was aspirated and the cells were resuspended in 50 milliliters of fresh Growth Medium. This process was repeated twice more.

Upon the final centrifugation, supernatant was aspirated and the cell pellet was resuspended in 5 milliliters of fresh Growth Medium. The number of viable cells was determined using Trypan Blue staining. Cells were then cultured under standard conditions.

The cells isolated from umbilical cords were seeded at 5,000 cells/cm² onto gelatin-coated T-75 cm² flasks (Corning Inc., Corning, N.Y.) in Growth Medium with antibiotics/antimycotics as described above. After 2 days (in various experiments, cells were incubated from 2-4 days), spent medium was aspirated from the flasks. Cells were washed with PBS three times to remove debris and blood-derived cells. Cells were then replenished with Growth Medium and allowed to grow to confluence (about 10 days from passage 0) to passage 1. On subsequent passages (from passage 1 to 2 and so on), cells reached sub-confluence (75-85 percent confluence) in 4-5 days. For these subsequent passages, cells were seeded at 5000 cells/cm². Cells were grown in a humidified incubator with 5 percent carbon dioxide and atmospheric oxygen, at 37° C.

Placental Cell Isolation:

Placental tissue was obtained from NDRI (Philadelphia, Pa.). The tissues were from a pregnancy and were obtained at the time of a normal surgical delivery. Placental cells were isolated as described for umbilical cell isolation.

The following example applies to the isolation of separate populations of maternal-derived and neonatal-derived cells from placental tissue.

The cell isolation protocol was performed aseptically in a laminar flow hood. The placental tissue was washed in phosphate buffered saline (PBS; Invitrogen, Carlsbad, Calif.) in the presence of antimycotic and antibiotic (as described above) to remove blood and debris. The placental tissue was then dissected into three sections: top-line (neonatal side or aspect), mid-line (mixed cell isolation neonatal and maternal) and bottom line (maternal side or aspect).

The separated sections were individually washed several times in PBS with antibiotic/antimycotic to further remove blood and debris. Each section was then mechanically dissociated in 150 cm² tissue culture plates in the presence of 50 milliliters of DMEM-Low glucose, to a fine pulp. The pulp was transferred to 50 milliliter conical tubes. Each tube contained approximately 5 grams of tissue. The tissue was digested in either DMEM-Low glucose or DMEM-High glucose medium containing antimycotic and antibiotic (100 U/milliliter penicillin, 100 micrograms/milliliter streptomycin, 0.25 micrograms/milliliter amphotericin B) and digestion enzymes. In some experiments an enzyme mixture of collagenase and dispase (“C:D”) was used containing collagenase (Sigma, St Louis, Mo.) at 500 Units/milliliter and dispase (Invitrogen) at 50 Units/milliliter in DMEM-Low glucose medium. In other experiments a mixture of collagenase, dispase and hyaluronidase (C:D:H) was used (collagenase, 500 Units/milliliter; dispase, 50 Units/milliliter; and hyaluronidase (Sigma), 5 Units/milliliter in DMEM-Low glucose). The conical tubes containing the tissue, medium, and digestion enzymes were incubated for 2 h at 37° C. in an orbital shaker (Environ, Brooklyn, N.Y.) at 225 rpm.

After digestion, the tissues were centrifuged at 150×g for 5 minutes, the resultant supernatant was aspirated off. The pellet was resuspended in 20 milliliters of Growth Medium with penicillin/streptomycin/amphotericin B. The cell suspension was filtered through a 70 micometer nylon cell strainer (BD Biosciences), chased by a rinse with an additional 5 milliliters of Growth Medium. The total cell suspension was passed through a 40 micometer nylon cell strainer (BD Biosciences) followed with an additional 5 milliliters of Growth Medium as a rinse.

The filtrate was resuspended in Growth Medium (total volume 50 milliliters) and centrifuged at 150×g for 5 minutes. The supernatant was aspirated and the cell pellet was resuspended in 50 milliliters of fresh Growth Medium. This process was repeated twice more. After the final centrifugation, supernatant was aspirated and the cell pellet was resuspended in 5 milliliters of fresh Growth Medium. A cell count was determined using the Trypan Blue Exclusion test. Cells were then cultured at standard conditions.

LIBERASE Cell Isolation:

Cells were isolated from umbilicus tissues in DMEM-Low glucose medium with LIBERASE (Boehringer Mannheim Corp., Indianapolis, Ind.) (2.5 milligrams per milliliter, Blendzyme 3; Roche Applied Sciences, Indianapolis, Ind.) and hyaluronidase (5 Units/milliliter, Sigma). Digestion of the tissue and isolation of the cells was as described for other protease digestions above, using the LIBERASE/hyaluronidase mixture in place of the C:D or C:D:H enzyme mixture. Tissue digestion with LIBERASE resulted in the isolation of cell populations from postpartum tissues that expanded readily.

Cell Isolation Using Other Enzyme Combinations:

Procedures were compared for isolating cells from the umbilical cord using differing enzyme combinations. Enzymes compared for digestion included: i) collagenase; ii) dispase; iii) hyaluronidase; iv) collagenase: dispase mixture (C:D); v) collagenase: hyaluronidase mixture (C:H); vi) dispase: hyaluronidase mixture (D:H); and vii) collagenase: dispase: hyaluronidase mixture (C:D:H). Differences in cell isolation utilizing these different enzyme digestion conditions were observed (Table 4-1).

Isolation of Cells from Residual Blood in the Cords:

Other attempts were made to isolate pools of cells from umbilical cord by different approaches. In one instance umbilical cord was sliced and washed with Growth Medium to dislodge the blood clots and gelatinous material. The mixture of blood, gelatinous material and Growth Medium was collected and centrifuged at 150×g. The pellet was resuspended and seeded onto gelatin-coated flasks in Growth Medium. From these experiments a cell population was isolated that readily expanded.

Isolation of Cells from Cord Blood:

Cells have also been isolated from cord blood samples attained from NDR1. The isolation protocol used here was that of International Patent Application WO 2003/025149 by Ho et al. (Ho, T. W., et al., “Cell Populations Which Co-Express CD49C and CD90,” Application No. PCT/US02/29971). Samples (50 milliliter and 10.5 milliliters, respectively) of umbilical cord blood (NDR1, Philadelphia Pa.) were mixed with lysis buffer (filter-sterilized 155 mM ammonium chloride, 10 millimolar potassium bicarbonate, 0.1 millimolar EDT A buffered to pH 7.2 (all components from Sigma, St. Louis, Mo.)). Cells were lysed at a ratio of 1:20 cord blood to lysis buffer. The resulting cell suspension was vortexed for 5 seconds, and incubated for 2 minutes at ambient temperature. The lysate was centrifuged (10 minutes at 200×g). The cell pellet was resuspended in complete minimal essential medium (Gibco, Carlsbad, Calif.) containing 10 percent fetal bovine serum (Hyclone, Logan Utah), 4 millimolar glutamine (Mediatech, Herndon, Va.), 100 Units penicillin per 100 milliliters and 100 micrograms streptomycin per 100 milliliters (Gibco, Carlsbad, Calif.). The resuspended cells were centrifuged (10 minutes at 200×g), the supernatant was aspirated, and the cell pellet was washed in complete medium. Cells were seeded directly into either T75 flasks (Corning, N.Y.), T75 laminin-coated flasks, or T175 fibronectin-coated flasks (both Becton Dickinson, Bedford, Mass.).

Isolation of Cells Using Different Enzyme Combinations and Growth Conditions:

To determine whether cell populations could be isolated under different conditions and expanded under a variety of conditions immediately after isolation, cells were digested in Growth Medium with or without 0.001 percent (v/v) 2-mercaptoethanol (Sigma, St. Louis, Mo.), using the enzyme combination of C:D:H, according to the procedures provided above. Placental-derived cells so isolated were seeded under a variety of conditions. All cells were grown in the presence of penicillin/streptomycin. (Table 4-2).

Isolation of Cells Using Different Enzyme Combinations and Growth Conditions:

In all conditions cells attached and expanded well between passage 0 and 1 (Table 4-2). Cells in conditions 5-8 and 13-16 were demonstrated to proliferate well up to 4 passages after seeding at which point they were cryopreserved and banked.

### Results

Cell Isolation Using Different Enzyme Combinations:

The combination of C:D:H, provided the best cell yield following isolation, and generated cells, which expanded for many more generations in culture than the other conditions (Table 4-1). An expandable cell population was not attained using collagenase or hyaluronidase alone. No attempt was made to determine if this result is specific to the collagen that was tested.

TABLE 4-1
Isolation of cells from umbilical cord tissue using varying
enzyme combinations

| Enzyme Digest | Cells Isolated | Cell Expansion |
|---|---|---|
| Collagenase | X | X |
| Dispase | +(>10 h) | + |
| Hyaluronidase | X | X |
| Collagenase:Dispase | ++(<3 h) | ++ |
| Collagenase:Hyaluronidase | ++(<3 h) | + |
| Dispase:Hyaluronidase | +(>10 h) | + |
| Collagenase:Dispase:Hyaluronidase | +++ (<3 h) | +++ |

Key: + = good,
++ = very good,
+++ = excellent,
X = no success

Isolation of Cells Using Different Enzyme Combinations and Growth Conditions:

Cells attached and expanded well between passage 0 and 1 under all conditions tested for enzyme digestion and growth (Table 4-2). Cells in experimental conditions 5-8 and 13-16 proliferated well up to 4 passages after seeding, at which point they were cryopreserved. All cells were cryopreserved for further investigation.

TABLE 4-2
Isolation and culture expansion of postpartum cells under varying conditions:

| Condition | Medium | 15% FBS | BME | Gelatin | 20% O₂ | Growth Factors |
|---|---|---|---|---|---|---|
| 1 | DMEM-Lg | Y | Y | Y | Y | N |
| 2 | DMEM-Lg | Y | Y | Y | N (5%) | N |
| 3 | DMEM-Lg | Y | Y | N | Y | N |
| 4 | DMEM-Lg | Y | Y | N | N (5%) | N |
| 5 | DMEM-Lg | N (2%) | Y | N (Laminin) | Y | EGF/FGF (20 ng/ml) |
| 6 | DMEM-Lg | N (2%) | Y | N (Laminin) | N (5%) | EGF/FGF (20 ng/ml) |
| 7 | DMEM-Lg | N (2%) | Y | N | Y | PDGF/VEGF |
| | | | | (Fibronectin) | | |
| 8 | DMEM-Lg | N (2%) | Y | N | N (5%) | PDGF/VEGF |
| | | | | (Fibronectin) | | |
| 9 | DMEM-Lg | Y | N | Y | Y | N |
| 10 | DMEM-Lg | Y | N | Y | N (5%) | N |
| 11 | DMEM-Lg | Y | N | N | Y | N |
| 12 | DMEM-Lg | Y | N | N | N (5%) | N |
| 13 | DMEM-Lg | N (2%) | N | N (Laminin) | Y | EGF/FGF (20 ng/ml) |
| 14 | DMEM-Lg | N (2%) | N | N (Laminin) | N (5%) | EGF/FGF (20 ng/ml) |
| 15 | DMEM-Lg | N (2%) | N | N | Y | PDGF/VEGF |
| | | | | (Fibronectin) | | |
| 16 | DMEM-Lg | N (2%) | N | N | N (5%) | PDGF/VEGF |
| | | | | (Fibronectin) | | |

Isolation of Cells from Residual Blood in the Cords:

Nucleated cells attached and grew rapidly. These cells were analyzed by flow cytometry and were similar to cells obtained by enzyme digestion.

Isolation of Cells from Cord Blood:

The preparations contained red blood cells and platelets. No nucleated cells attached and divided during the first 3 weeks. The medium was changed 3 weeks after seeding and no cells were observed to attach and grow.

Summary:

Populations of cells can be derived from umbilical cord and placental tissue efficiently using the enzyme combination collagenase (a matrix metalloprotease), dispase (a neutral protease) and hyaluronidase (a mucolytic enzyme that breaks down hyaluronic acid). LIBERASE, which is a Blendzyme, may also be used. Specifically, Blendzyme 3, which is collagenase (4 Wunsch units/g) and thermolysin (1714 casein Units/g) was also used together with hyaluronidase to isolate cells. These cells expanded readily over many passages when cultured in Growth Medium on gelatin-coated plastic.

Cells were also isolated from residual blood in the cords, but not cord blood. The presence of cells in blood clots washed from the tissue that adhere and grow under the conditions used may be due to cells being released during the dissection process.

### Example 5

### Karyotype Analysis of Postpartum-Derived Cells

Cell lines used in cell therapy are preferably homogeneous and free from any contaminating cell type. Cells used in cell therapy should have a normal chromosome number (46) and structure. To identify placenta- and umbilicus-derived cell lines that are homogeneous and free from cells of non-postpartum tissue origin, karyotypes of cell samples were analyzed.

### Methods & Materials

PPDCs from postpartum tissue of a male neonate were cultured in Growth Medium containing penicillin/streptomycin. Postpartum tissue from a male neonate (X,Y) was selected to allow distinction between neonatal-derived cells and maternal derived cells (X,X). Cells were seeded at 5,000 cells per square centimeter in Growth Medium in a T25 flask (Corning Inc., Corning, N.Y.) and expanded to 80% confluence. A T25 flask containing cells was filled to the neck with Growth Medium. Samples were delivered to a clinical cytogenetics laboratory by courier (estimated lab to lab transport time is one hour). Cells were analyzed during metaphase when the chromosomes are best visualized. Of twenty cells in metaphase counted, five were analyzed for normal homogeneous karyotype number (two). A cell sample was characterized as homogeneous if two karyotypes were observed. A cell sample was characterized as heterogeneous if more than two karyotypes were observed. Additional metaphase cells were counted and analyzed when a heterogeneous karyotype number (four) was identified.

### Results

All cell samples sent for chromosome analysis were interpreted as exhibiting a normal appearance. Three of the 16 cell lines analyzed exhibited a heterogeneous phenotype (XX and XY) indicating the presence of cells derived from both neonatal and maternal origins (Table 5-1). Cells derived from tissue Placenta-N were isolated from the neonatal aspect of placenta. At passage zero, this cell line appeared homogeneous XY. However, at passage nine, the cell line was heterogeneous (XX/XY), indicating a previously undetected presence of cells of maternal origin.

TABLE 5-1
Karyotype results of PPDCs.

| | | Metaphase | Metaphase | Number | |
|---|---|---|---|---|---|
| | | cells | cells | of | ISCN |
| Tissue | passage | counted | analyzed | karyotypes | Karyotype |
| Placenta | 22 | 20 | 5 | 2 | 46, XX |
| Umbilical | 23 | 20 | 5 | 2 | 46, XX |
| Umbilical | 6 | 20 | 5 | 2 | 46, XY |
| Placenta | 2 | 20 | 5 | 2 | 46, XX |
| Umbilical | 3 | 20 | 5 | 2 | 46, XX |
| Placenta-N | 0 | 20 | 5 | 2 | 46, XY |
| Placenta-V | 0 | 20 | 5 | 2 | 46, XY |
| Placenta-M | 0 | 21 | 5 | 4 | 46, XY[18]/46, |
| | | | | | XX[3] |
| Placenta-M | 4 | 20 | 5 | 2 | 46, XX |
| Placenta-N | 9 | 25 | 5 | 4 | 46, XY[5]/46, |
| | | | | | XX[20] |
| Placenta-N | 1 | 20 | 5 | 2 | 46, XY |
| Cl | | | | | |
| Placenta-N | 1 | 20 | 6 | 4 | 46, XY[2]/46, |
| C3 | | | | | XX[18] |
| Placenta-N | 1 | 20 | 5 | 2 | 46, XY |
| C4 | | | | | |
| Placenta-N | 1 | 20 | 5 | 2 | 46, XY |
| C15 | | | | | |
| Placenta-N | 1 | 20 | 5 | 2 | 46, XY |
| C20 | | | | | |
| Placenta-N | 1 | 20 | 5 | 2 | 46, XY |
| C22 | | | | | |

Key: N-Neonatal side;
V-villous region;
M-maternal side;
C-clone

Summary:

Chromosome analysis identified placenta- and umbilicus-derived cells whose karyotypes appeared normal as interpreted by a clinical cytogenetic laboratory. Karyotype analysis also identified cell lines free from maternal cells, as determined by homogeneous karyotype.

### Example 6

### Evaluation of Human Postpartum-Derived Cell Surface Markers by Flow Cytometry

Characterization of cell surface proteins or “markers” by flow cytometry can be used to determine a cell line's identity. The consistency of expression can be determined from multiple donors, and in cells exposed to different processing and culturing conditions. Postpartum-derived cell (PPDC) lines isolated from the placenta and umbilicus were characterized (by flow cytometry), providing a profile for the identification of these cell lines.

### Methods & Materials

Media and Culture Vessels:

Cells were cultured in Growth Medium (Gibco Carlsbad, Calif.) with penicillin/streptomycin. Cells were cultured in plasma-treated T75, T150, and T225 tissue culture flasks (Corning Inc., Corning, N.Y.) until confluent. The growth surfaces of the flasks were coated with gelatin by incubating 2% (w/v) gelatin (Sigma, St. Louis, Mo.) for 20 minutes at room temperature.

Antibody Staining and Flow Cytometry Analysis:

Adherent cells in flasks were washed in PBS and detached with Trypsin/EDTA. Cells were harvested, centrifuged, and resuspended in 3% (v/v) FBS in PBS at a cell concentration of 1×10⁷ per milliliter. In accordance to the manufacture's specifications, antibody to the cell surface marker of interest (see below) was added to one hundred microliters of cell suspension and the mixture was incubated in the dark for 30 minutes at 4° C. After incubation, cells were washed with PBS and centrifuged to remove unbound antibody. Cells were resuspended in 500 microliter PBS and analyzed by flow cytometry. Flow cytometry analysis was performed with a FACScalibur™ instrument (Becton Dickinson, San Jose, Calif.). Table 6-1 lists the antibodies to cell surface markers that were used.

TABLE 6-1
Antibodies used in characterizing cell surface markers.

| | | Catalog |
|---|---|---|
| Antibody | Manufacture | Number |
| CD10 | BD Pharmingen (San Diego, CA) | 555375 |
| CD13 | BD Pharmingen (San Diego, CA) | 555394 |
| CD31 | BD Pharmingen (San Diego, CA) | 555446 |
| CD34 | BD Pharmingen (San Diego, CA) | 555821 |
| CD44 | BD Pharmingen (San Diego, CA) | 555478 |
| CD45RA | BD Pharmingen (San Diego, CA) | 555489 |
| CD73 | BD Pharmingen (San Diego, CA) | 550257 |
| CD90 | BD Pharmingen (San Diego, CA) | 555596 |
| CD117 | BD Biosciences (San Jose, CA) | 340529 |
| CD141 | BD Pharmingen (San Diego, CA) | 559781 |
| PDGFr-alpha | BD Pharmingen (San Diego, CA) | 556002 |
| HLA-A, B, C | BD Pharmingen (San Diego, CA) | 555553 |
| HLA-DR, DP, DQ | BD Pharmingen (San Diego, CA) | 555558 |
| IgG-FITC | Sigma (St. Louis, MO) | F-6522 |
| IgG- PE | Sigma (St. Louis, MO) | P-4685 |

Placenta and Umbilicus Comparison:

Placenta-derived cells were compared to umbilicus-derive cells at passage 8.

Passage to Passage Comparison:

Placenta- and umbilicus-derived cells were analyzed at passages 8, 15, and 20.

Donor to Donor Comparison:

To compare differences among donors, placenta-derived cells from different donors were compared to each other, and umbilicus-derived cells from different donors were compared to each other.

Surface Coating Comparison:

Placenta-derived cells cultured on gelatin-coated flasks was compared to placenta-derived cells cultured on uncoated flasks. Umbilicus-derived cells cultured on gelatin-coated flasks was compared to umbilicus-derived cells cultured on uncoated flasks.

Digestion Enzyme Comparison:

Four treatments used for isolation and preparation of cells were compared. Cells isolated from placenta by treatment with 1) collagenase; 2) collagenase/dispase; 3) collagenase/hyaluronidase; and 4) collagenase/hyaluronidase/dispase were compared.

Placental Layer Comparison:

Cells derived from the maternal aspect of placental tissue were compared to cells derived from the villous region of placental tissue and cells derived from the neonatal fetal aspect of placenta.

### Results

Placenta Vs. Umbilicus Comparison:

Placenta- and umbilicus-derived cells analyzed by flow cytometry showed positive expression of CD10, CD13, CD44, CD73, CD90, PDGFr-alpha and HLA-A, B, C, indicated by the increased values of fluorescence relative to the IgG control. These cells were negative for detectable expression of CD31, CD34, CD45, CD117, CD141, and HLA-DR, DP, DQ, indicated by fluorescence values comparable to the IgG control. Variations in fluorescence values of positive curves were accounted. The mean (i.e. CD13) and range (i.e. CD90) of the positive curves showed some variation, but the curves appeared normal, confirming a homogenous population. Both curves individually exhibited values greater than the IgG control.

Passage to Passage Comparison—Placenta-Derived Cells:

Placenta-derived cells at passages 8, 15, and 20 analyzed by flow cytometry all were positive for expression of CD10, CD13, CD44, CD73, CD90, PDGFr-alpha and HLA-A, B, C, as reflected in the increased value of fluorescence relative to the IgG control. The cells were negative for expression of CD31, CD34, CD45, CD117, CD141, and HLA-DR, DP, DQ having fluorescence values consistent with the IgG control.

Passage to Passage Comparison—Umbilicus-Derived Cells:

Umbilicus-derived cells at passage 8, 15, and 20 analyzed by flow cytometry all expressed CD10, CD13, CD44, CD73, CD90, PDGFr-alpha and HLA-A, B, C, indicated by increased fluorescence relative to the IgG control. These cells were negative for CD31, CD34, CD45, CD117, CD141, and HLA-DR, DP, DQ, indicated by fluorescence values consistent with the IgG control.

Donor to Donor Comparison—Placenta-Derived Cells:

Placenta-derived cells isolated from separate donors analyzed by flow cytometry each expressed CD10, CD13, CD44, CD73, CD90, PDGFr-alpha and HLA-A, B, C, with increased values of fluorescence relative to the IgG control. The cells were negative for expression of CD31, CD34, CD45, CD117, CD141, and HLA-DR, DP, DQ as indicated by fluorescence value consistent with the IgG control.

Donor to Donor Comparison—Umbilicus Derived Cells:

Umbilicus-derived cells isolated from separate donors analyzed by flow cytometry each showed positive expression of CD10, CD13, CD44, CD73, CD90, PDGFr-alpha and HLA-A, B, C, reflected in the increased values of fluorescence relative to the IgG control. These cells were negative for expression of CD31, CD34, CD45, CD117, CD141, and HLA-DR, DP, DQ with fluorescence values consistent with the IgG control.

The Effect of Surface Coating with Gelatin on Placenta-Derived Cells:

Placenta-derived cells expanded on either gelatin-coated or uncoated flasks analyzed by flow cytometry all expressed of CD10, CD13, CD44, CD73, CD90, PDGFr-alpha and HLA-A, B, C, reflected in the increased values of fluorescence relative to the IgG control. These cells were negative for expression of CD31, CD34, CD45, CD117, CD141, and HLA-DR, DP, DQ indicated by fluorescence values consistent with the IgG control.

The Effect of Surface Coating with Gelatin on Umbilicus-Derived Cells:

Umbilicus-derived cells expanded on gelatin and uncoated flasks analyzed by flow cytometry all were positive for expression of CD10, CD13, CD44, CD73, CD90, PDGFr-alpha and HLA-A, B, C, with increased values of fluorescence relative to the IgG control. These cells were negative for expression of CD31, CD34, CD45, CD117, CD141, and HLA-DR, DP, DQ, with fluorescence values consistent with the IgG control.

Effect of Enzyme Digestion Procedure Used for Preparation of the Cells on the Cell Surface Marker Profile:

Placenta-derived cells isolated using various digestion enzymes analyzed by flow cytometry all expressed CD10, CD13, CD44, CD73, CD90, PDGFr-alpha and HLA-A, B, C, as indicated by the increased values of fluorescence relative to the IgG control. These cells were negative for expression of CD31, CD34, CD45, CD117, CD141, and HLADR, DP, DQ as indicated by fluorescence values consistent with the IgG control.

Placental Layer Comparison:

Cells isolated from the maternal, villous, and neonatal layers of the placenta, respectively, analyzed by flow cytometry showed positive expression of CD10, CD13, CD44, CD73, CD90, PDGFr-alpha and HLA-A, B, C, as indicated by the increased value of fluorescence relative to the IgG control. These cells were negative for expression of CD31, CD34, CD45, CD117, CD141, and HLA-DR, DP, DQ as indicated by fluorescence values consistent with the IgG control.

Summary:

Analysis of placenta- and umbilicus-derived cells by flow cytometry has established of an identity of these cell lines. Placenta- and umbilicus-derived cells are positive for CD10, CD13, CD44, CD73, CD90, PDGFr-alpha, HLA-A,B,C and negative for CD31, CD34, CD45, CD117, CD141 and HLA-DR, DP, DQ. This identity was consistent between variations in variables including the donor, passage, culture vessel surface coating, digestion enzymes, and placental layer. Some variation in individual fluorescence value histogram curve means and ranges was observed, but all positive curves under all conditions tested were normal and expressed fluorescence values greater than the IgG control, thus confirming that the cells comprise a homogenous population that has positive expression of the markers.

### Example 7

### Immunohistochemical Characterization of Postpartum Tissue Phenotypes

The phenotypes of cells found within human postpartum tissues, namely umbilical cord and placenta, was analyzed by immunohistochemistry.

### Methods & Materials

Tissue Preparation:

Human umbilical cord and placenta tissue was harvested and immersion fixed in 4% (w/v) paraformaldehyde overnight at 4° C. Immunohistochemistry was performed using antibodies directed against the following epitopes: vimentin (1:500; Sigma, St. Louis, Mo.), desmin (1:150, raised against rabbit; Sigma; or 1:300, raised against mouse; Chemic on, Temecula, Calif.), alpha-smooth muscle actin (SMA; 1:400; Sigma), cytokeratin 18 (CK18; 1:400; Sigma), von Willebrand Factor (vWF; 1:200; Sigma), and CD34 (human CD34 Class III; 1:100; DAKOCytomation, Carpinteria, Calif.). In addition, the following markers were tested: antihuman GROalpha-PE (1:100; Becton Dickinson, Franklin Lakes, N.J), antihuman GCP-2 (1:100; Santa Cruz Biotech, Santa Cruz, Calif.), anti-human oxidized LDL receptor 1 (ox-LDL R1; 1:100; Santa Cruz Biotech), and anti-human NOGO-A (1:100; Santa Cruz Biotech). Fixed specimens were trimmed with a scalpel and placed within OCT embedding compound (Tissue-Tek OCT; Sakura, Torrance, Calif.) on a dry ice bath containing ethanol. Frozen blocks were then sectioned (10 μm thick) using a standard cryostat (Leica Microsystems) and mounted onto glass slides for staining.

Immunohistochemistry:

Immunohistochemistry was performed similar to previous studies (e.g., Messina, et al., 2003, Exper. Neurol. 184: 816-829). Tissue sections were washed with phosphate-buffered saline (PBS) and exposed to a protein blocking solution containing PBS, 4% (v/v) goat serum (Chemic on, Temecula, Calif.), and 0.3% (v/v) Triton (Triton X-100; Sigma) for 1 hour to access intracellular antigens. In instances where the epitope of interest would be located on the cell surface (CD34, ox-LDL R1), Triton was omitted in all steps of the procedure in order to prevent epitope loss. Furthermore, in instances where the primary antibody was raised against goat (GCP-2, ox-LDL R1, NOGO-A), 3% (v/v) donkey serum was used in place of goat serum throughout the procedure. Primary antibodies, diluted in blocking solution, were then applied to the sections for a period of 4 hours at room temperature. Primary antibody solutions were removed, and cultures washed with PBS prior to application of secondary antibody solutions (1 hour at room temperature) containing block along with goat anti-mouse IgG--Texas Red (1:250; Molecular Probes, Eugene, Oreg.) and/or goat anti-rabbit IgG--Alexa 488 (1:250; Molecular Probes) or donkey anti-goat IgG--FITC (1:150; Santa Cruz Biotech). Cultures were washed, and 10 micromolar DAPI (Molecular Probes) was applied for 10 minutes to visualize cell nuclei.

Following immunostaining, fluorescence was visualized using the appropriate fluorescence filter on an Olympus inverted epi-fluorescent microscope (Olympus, Melville, N.Y.). Positive staining was represented by fluorescence signal above control staining. Representative images were captured using a digital color video camera and ImagePro software (Media Cybernetics, Carlsbad, Calif.). For triple-stained samples, each image was taken using only one emission filter at a time. Layered montages were then prepared using Adobe Photoshop software (Adobe, San Jose, Calif.).

### Results

Umbilical Cord Characterization:

Vimentin, desmin, SMA, CKI8, vWF, and CD34 markers were expressed in a subset of the cells found within umbilical cord. In particular, vWF and CD34 expression were restricted to blood vessels contained within the cord. CD34+ cells were on the innermost layer (lumen side). Vimentin expression was found throughout the matrix and blood vessels of the cord. SMA was limited to the matrix and outer walls of the artery & vein, but not contained with the vessels themselves. CK18 and desmin were observed within the vessels only, desmin being restricted to the middle and outer layers.

Placenta Characterization:

Vimentin, desmin, SMA, CKI8, vWF, and CD34 were all observed within the placenta and regionally specific.

GROalpha, GCP-2, ox-LDL RI, and NOGO-A Tissue Expression:

None of these markers were observed within umbilical cord or placental tissue.

Summary:

Vimentin, desmin, alpha-smooth muscle actin, cytokeratin 18, von Willebrand Factor, and CD34 are expressed in cells within human umbilical cord and placenta.

### Example 8

### Analysis of Postpartum Tissue-Derived Cells using Oligonucleotide Arrays

Affymetrix GENECHIP arrays were used to compare gene expression profiles of umbilicus- and placenta-derived cells with fibroblasts, human mesenchymal stem cells, and another cell line derived from human bone marrow. This analysis provided a characterization of the postpartum-derived cells and identified unique molecular markers for these cells.

### Methods & Materials

Isolation and Culture of Cells:

Human umbilical cords and placenta were obtained from National Disease Research Interchange (NDRI, Philadelphia, Pa.) from normal full term deliveries with patient consent. The tissues were received and cells were isolated as described in Example 6. Cells were cultured in Growth Medium (using DMEM-LG) on gelatin-coated tissue culture plastic flasks. The cultures were incubated at 37° C. with 5% CO₂.

Human dermal fibroblasts were purchased from Cambrex Incorporated (Walkersville, Md.; Lot number 9F0844) and ATCC CRL-1501 (CCD39SK). Both lines were cultured in DMEM/F12 medium (Invitrogen, Carlsbad, Calif.) with 10% (v/v) fetal bovine serum (Hyclone) and penicillin/streptomycin (Invitrogen). The cells were grown on standard tissue-treated plastic.

Human mesenchymal stem cells (hMSC) were purchased from Cambrex Incorporated (Walkersville, Md.; Lot numbers 2F1655, 2F1656 and 2F1657) and cultured according to the manufacturer's specifications in MSCGM Media (Cambrex). The cells were grown on standard tissue cultured plastic at 37° C. with 5% CO₂.

Human iliac crest bone marrow was received from the NDRI with patient consent. The marrow was processed according to the method outlined by Ho, et al. (WO03/025149). The marrow was mixed with lysis buffer (155 mM NH 4Cl, 10 mM KHCO₃, and 0.1 mM EDTA, pH 7.2) at a ratio of 1 part bone marrow to 20 parts lysis buffer. The cell suspension was vortexed, incubated for 2 minutes at ambient temperature, and centrifuged for 10 minutes at 500×g. The supernatant was discarded and the cell pellet was resuspended in Minimal Essential Medium-alpha (Invitrogen) supplemented with 10% (v/v) fetal bovine serum and 4 mM glutamine. The cells were centrifuged again and the cell pellet was resuspended in fresh medium. The viable mononuclear cells were counted using trypan-blue exclusion (Sigma, St. Louis, Mo.). The mononuclear cells were seeded in tissue-cultured plastic flasks at 5×10⁴ cells/cm². The cells were incubated at 37° C. with 5% CO₂ at either standard atmospheric O₂ or at 5% O₂. Cells were cultured for 5 days without a media change. Media and non-adherent cells were removed after 5 days of culture. The adherent cells were maintained in culture.

Isolation of mRNA and GENECHIP Analysis:

Actively growing cultures of cells were removed from the flasks with a cell scraper in cold PBS. The cells were centrifuged for 5 minutes at 300×g. The supernatant was removed and the cells were resuspended in fresh PBS and centrifuged again. The supernatant was removed and the cell pellet was immediately frozen and stored at −80° C. Cellular mRNA was extracted and transcribed into cDNA, which was then transcribed into cRNA and biotin-labeled. The biotin-labeled cRNA was hybridized with HG-U133A GENECHIP oligonucleotide array (Affymetrix, Santa Clara Calif.). The hybridization and data collection was performed according to the manufacturer's specifications. Analyses were performed using “Significance Analysis of Microarrays” (SAM) version 1.21 computer software (Stanford University; Tusher, V. G. et al., 2001, Proc. Natl. Acad. Sci. USA 98: 5116-5121).\

### Results

Fourteen different populations of cells were analyzed. The cells along with passage information, culture substrate, and culture media are listed in Table 8-1.

TABLE 8-1
Cells analyzed by the microarray study. The cells
lines are listed by their identification code along with passage
at the time of analysis, cell growth substrate, and growth media.

| Cell Population | Passage | Substrate | Medium |
|---|---|---|---|
| Umbilical (022803) | 2 | Gelatin | DMEM, 15% FBS, 2-ME |
| Umbilical (042103) | 3 | Gelatin | DMEM, 15% FBS, 2-ME |
| Umbilical (071003) | 4 | Gelatin | DMEM, 15% FBS, 2-ME |
| Placenta (042203) | 12 | Gelatin | DMEM, 15% FBS, 2-ME |
| Placenta (042903) | 4 | Gelatin | DMEM, 15% FBS, 2-ME |
| Placenta (071003) | 3 | Gelatin | DMEM, 15% FBS, 2-ME |
| ICBM (070203) (5% O₂) | 3 | Plastic | MEM 10% FBS |
| ICBM (062703) (std O₂) | 5 | Plastic | MEM 10% FBS |
| ICBM (062703 )(5% O₂) | 5 | Plastic | MEM 10% FBS |
| hMSC (Lot 2F1655) | 3 | Plastic | MSCGM |
| hMSC (Lot 2F1656) | 3 | Plastic | MSCGM |
| hMSC (Lot 2F1657) | 3 | Plastic | MSCGM |
| hFibroblast (9F0844) | 9 | Plastic | DMEM-F12, 10% FBS |
| hFibroblast (CCD39SK) | 4 | Plastic | DMEM-F12, 10% FBS |

The data were evaluated by a Principle Component Analysis, analyzing the 290 genes that were differentially expressed in the cells. This analysis allows for a relative comparison for the similarities between the populations.

Table 8-2 shows the Euclidean distances that were calculated for the comparison of the cell pairs. The Euclidean distances were based on the comparison of the cells based on the 290 genes that were differentially expressed among the cell types. The Euclidean distance is inversely proportional to similarity between the expression of the 290 genes (i.e., the greater the distance, the less similarity exists).

TABLE 8-2
The Euclidean Distances for the Cell Pairs.

| Cell Pair | Euclidean Distance |
|---|---|
| ICBM-hMSC | 24.71 |
| Placenta-umbilical | 25.52 |
| ICBM-Fibroblast | 36.44 |
| ICBM-placenta | 37.09 |
| Fibroblast-MSC | 39.63 |
| ICBM-Umbilical | 40.15 |
| Fibroblast-Umbilical | 41.59 |
| MSC-Placenta | 42.84 |
| MSC-Umbilical | 46.86 |
| ICBM-placenta | 48.41 |

Tables 8-3, 8-4, and 8-5 show the expression of genes increased in placenta-derived cells (Table 8-3), increased in umbilicus-derived cells (Table 8-4), and reduced in umbilicus- and placenta-derived cells (Table 8-5). The column entitled “Probe Set ID” refers to the manufacturer's identification code for the sets of several oligonucleotide probes located on a particular site on the chip, which hybridize to the named gene (column “Gene Name”), comprising a sequence that can be found within the NCBI (GenBank) database at the specified accession number (column “NCBI Accession Number”).

TABLE 8-3
Genes shown to have specifically increased expression in the
placenta-derived cells as compared to other cell lines assayed
Genes Increased in Placenta-Derived Cells

| | | NCBI |
|---|---|---|
| Probe | | Accession |
| Set ID | Gene Name | Number |
| 209732_at | C-type (calcium dependent, | AF070642 |
| | carbohydrate-recognition domain) lectin, | |
| | superfamily member 2 (activation-induced) | |
| 206067_s_at | Wilms tumor 1 | NM_024426 |
| 207016_s_at | aldehyde dehydrogenase 1 family, member A2 | AB015228 |
| 206367_at | renin | NM_000537 |
| 210004_at | oxidized low density lipoprotein | AF035776 |
| | (lectin-like) receptor 1 | |
| 214993_at | Homo sapiens, clone IMAGE: 4179671, | AF070642 |
| | mRNA, partial cds | |
| 202178_at | protein kinase C, zeta | NM_002744 |
| 209780_at | hypothetical protein DKFZp564F013 | AL136883 |
| 204135_at | downregulated in ovarian cancer 1 | NM_014890 |
| 213542_at | Homo sapiens mRNA; cDNA | AI246730 |
| | DKFZp547K1113 | |
| | (from clone DKFZp547K1113) | |

TABLE 8-4
Genes shown to have specifically increased expression in the
umbilicus-derived cells as compared to other cell lines assayed
Genes Increased in Umbilicus-Derived Cells

| | | NCBI |
|---|---|---|
| | | Accession |
| Probe Set ID | Gene Name | Number |
| 202859_x_at | interleukin 8 | NM_000584 |
| 211506_s_at | interleukin 8 | AF043337 |
| 210222_s_at | reticulon 1 | BC000314 |
| 204470_at | chemokine (C-X-C motif) ligand 1 | NM_001511 |
| | (melanoma growth stimulating activity | |
| 206336_at | chemokine (C-X-C motif) ligand 6 | NM_002993 |
| | (granulocyte chemotactic protein 2) | |
| 207850_at | chemokine (C-X-C motif) ligand 3 | NM_002090 |
| 203485_at | reticulon 1 | NM_021136 |
| 202644_s_at | tumor necrosis factor, alpha-induced protein 3 | NM_006290 |

TABLE 8-5
Genes shown to have decreased expression in umbilicus- and placenta-derived cells as compared
to other cell lines assayed
Genes Decreased in Umbilicus-and Placenta-Derived Cells

| | | NCBI |
|---|---|---|
| | | Accession |
| Probe Set ID | Gene name | Number |
| 210135_s_at | short stature homeobox 2 | AF022654.1 |
| 205824_at | heat shock 27 kDa protein 2 | NM_001541.1 |
| 209687_at | chemokine (C-X-C motif) ligand 12 (stromal cell-derived factor 1) | U19495.1 |
| 203666_at | chemokine (C-X-C motif) ligand 12 (stromal cell-derived factor 1) | NM_000609.1 |
| 212670_at | elastin (supravalvular aortic stenosis, Williams-Beuren syndrome) | AA479278 |
| 213381_at | Homo sapiens mRNA; cDNA DKFZp586M2022 (from clone | N91149 |
| | DKFZp586M2022) | |
| 206201_s_at | mesenchyme homeo box 2 (growth arrest-specific homeo box) | NM_005924.1 |
| 205817_at | sine oculis homeobox homolog 1 (Drosophila) | NM_005982.1 |
| 209283_at | crystallin, alpha B | AF007162.1 |
| 212793_at | dishevelled associated activator of morphogenesis 2 | BF513244 |
| 213488_at | DKFZP586B2420 protein | AL050143.1 |
| 209763_at | similar to neuralin 1 | AL049176 |
| 205200_at | tetranectin (plasminogen binding protein) | NM_003278.1 |
| 205743_at | src homology three (SH3) and cysteine rich domain | NM_003149.1 |
| 200921_s_at | B-cell translocation gene 1, anti-proliferative | NM_001731.1 |
| 206932_at | cholesterol 25-hydroxylase | NM_003956.1 |
| 204198_s_at | runt-related transcription factor 3 | AA541630 |
| 219747_at | hypothetical protein FLJ23191 | NM_024574.1 |
| 204773_at | interleukin 11 receptor, alpha | NM_004512.1 |
| 202465_at | procollagen C-endopeptidase enhancer | NM_002593.2 |
| 203706_s_at | frizzled homolog 7 (Drosophila) | NM_003507.1 |
| 212736_at | hypothetical gene BC008967 | BE299456 |
| 214587_at | collagen, type VIII, alpha 1 | BE877796 |
| 201645_at | tenascin C (hexabrachion) | NM_002160.1 |
| 210239_at | iroquois homeobox protein 5 | U90304.1 |
| 203903_s_at | Hephaestin | NM_014799.1 |
| 205816_at | integrin, beta 8 | NM_002214.1 |
| 203069_at | synaptic vesicle glycoprotein 2 | NM_014849.1 |
| 213909_at | Homo sapiens cDNA FLJ12280 fis, clone MAMMA1001744 | AU147799 |
| 206315_at | cytokine receptor-like factor 1 | NM_004750.1 |
| 204401_at | potassium intermediate/small conductance calcium-activated | NM_002250.1 |
| | channel, subfamily N, member 4 | |
| 216331_at | integrin, alpha 7 | AK022548.1 |
| 209663_s_at | integrin, alpha 7 | AF072132.1 |
| 213125_at | DKFZP586L151 protein | AW007573 |
| 202133_at | transcriptional co-activator with PDZ-binding motif (TAZ) | AA081084 |
| 206511_s_at | sine oculis homeobox homolog 2 (Drosophila) | NM_016932.1 |
| 213435_at | KIAA1034 protein | AB028957.1 |
| 206115_at | early growth response 3 | NM_004430.1 |
| 213707_s_at | distal-less homeo box 5 | NM_005221.3 |
| 218181_s_at | hypothetical protein FLJ20373 | NM_017792.1 |
| 209160_at | aldo-keto reductase family 1, member C3 (3-alpha | AB018580.1 |
| | hydroxysteroid dehydrogenase, type II) | |
| 213905_x_at | Biglycan | AA845258 |
| 201261_x_at | Biglycan | BC002416.1 |
| 202132_at | transcriptional co-activator with PDZ-binding motif (TAZ) | AA081084 |
| 214701_s_at | fibronectin 1 | AJ276395.1 |
| 213791_at | Proenkephalin | NM_006211.1 |
| 205422_s_at | integrin, beta-like 1 (with EGF-like repeat domains) | NM_004791.1 |
| 214927_at | Homo sapiens mRNA full length insert cDNA clone | AL359052.1 |
| | EUROIMAGE 1968422 | |
| 206070_s_at | EphA3 | AF213459.1 |
| 212805_at | KIAA0367 protein | AB002365.1 |
| 219789_at | natriuretic peptide receptor C/guanylate cyclase C | AI628360 |
| | (atrionatriuretic peptide receptor C) | |
| 219054_at | hypothetical protein FLJ14054 | NM_024563.1 |
| 213429_at | Homo sapiens mRNA; cDNA DKFZp564B222 (from clone | AW025579 |
| | DKFZp564B222) | |
| 204929_s_at | vesicle-associated membrane protein 5 (myobrevin) | NM_006634.1 |
| 201843_s_at | EGF-containing fibulin-like extracellular matrix protein 1 | NM_004105.2 |
| 221478_at | BCL2/adenovirus E1B 19 kDa interacting protein 3-like | AL132665.1 |
| 201792_at | AE binding protein 1 | NM_001129.2 |
| 204570_at | cytochrome c oxidase subunit Vila polypeptide 1 (muscle) | NM_001864.1 |
| 201621_at | neuroblastoma, suppression of tumorigenicity 1 | NM_005380.1 |
| 202718_at | insulin-like growth factor binding protein 2, 36 kDa | NM_000597.1 |

Tables 8-6, 8-7, and 8-8 show the expression of genes increased in human fibroblasts (Table 8-6), ICBM cells (Table 8-7), and MSCs (Table 8-8).

TABLE 8-6
Genes that were shown to have increased expression in fibroblasts
as compared to the other cell lines assayed.
Genes increased in fibroblasts

dual specificity phosphatase 2
KIAA0527 protein
Homo sapiens cDNA: FLJ23224 fis, clone ADSU02206
dynein, cytoplasmic, intermediate polypeptide 1
ankyrin 3, node of Ranvier (ankyrin G)
inhibin, beta A (activin A, activin AB alpha polypeptide)
ectonucleotide pyrophosphatase/phosphodiesterase 4 (putative
function)
KIAA1053 protein
microtubule-associated protein 1A
zinc finger protein 41
HSPC019 protein
Homo sapiens cDNA: FLJ23564 fis, clone LNG10773
Homo sapiens mRNA; cDNA DKFZp564A072 (from clone
DKFZp564A072)
LIM protein (similar to rat protein kinase C-binding enigma)
inhibitor of kappa light polypeptide gene enhancer in B-cells,
kinase complex-associated protein
hypothetical protein FLJ22004
Human (clone CTG-A4) mRNA sequence
ESTs, Moderately similar to cytokine receptor-like factor 2; cytokine
receptor CRL2 precursor [Homo sapiens]
transforming growth factor, beta 2
hypothetical protein MGC29643
antigen identified by monoclonal antibody MRC OX-2
putative X-linked retinopathy protein

TABLE 8-7
Genes that were shown to have increased expression in the
ICBM-derived cells as compared to the other cell lines assayed.
Genes Increased In ICBM Cells

cardiac ankyrin repeat protein
MHC class I region ORF
integrin, alpha 10
hypothetical protein FLJ22362
UDP-N-acetyl-alpha-D-galactosamine:polypeptide
N-acetylgalactosaminyltransferase 3 (GalNAc-T3)
interferon-induced protein 44
SRY (sex determining region Y)-box 9 (campomelic dysplasia,
autosomal sex-reversal)
keratin associated protein 1-1
hippocalcin-like 1
jagged 1 (Alagille syndrome)
proteoglycan 1, secretory granule

TABLE 8-8
Genes that were shown to have increased expression in the
MSC cells as compared to the other cell lines assayed.
Genes Increased In MSC Cells

interleukin 26
maltase-glucoamylase (alpha-glucosidase)
nuclear receptor subfamily 4, group A, member 2
v-fos FBJ murine osteosarcoma viral oncogene homolog
hypothetical protein DC42
nuclear receptor subfamily 4, group A, member 2
FBJ murine osteosarcoma viral oncogene homolog B
WNT1 inducible signaling pathway protein 1
MCF.2 cell line derived transforming sequence
potassium channel, subfamily K, member 15
cartilage paired-class homeoprotein 1
Homo sapiens cDNA FLJ12232 fis, clone MAMMA1001206
Homo sapiens cDNA FLJ34668 fis, clone LIVER2000775
jun B proto-oncogene
B-cell CLL/lymphoma 6 (zinc finger protein 51)
zinc finger protein 36, C3H type, homolog (mouse)

Summary:

The present examination was performed to provide a molecular characterization of the postpartum cells derived from umbilical cord and placenta. This analysis included cells derived from three different umbilical cords and three different placentas. The examination also included two different lines of dermal fibroblasts, three lines of mesenchymal stem cells, and three lines of iliac crest bone marrow cells. The mRNA that was expressed by these cells was analyzed using an oligonucleotide array that contained probes for 22,000 genes. Results showed that 290 genes are differentially expressed in these five different cell types. These genes include ten genes that are specifically increased in the placenta-derived cells and seven genes specifically increased in the umbilical cord-derived cells. Fifty-four genes were found to have specifically lower expression levels in placenta and umbilical cord, as compared with the other cell types. The expression of selected genes has been confirmed by PCR (see the example that follows). These results demonstrate that the postpartum-derived cells have a distinct gene expression profile, for example, as compared to bone marrow-derived cells and fibroblasts.

### Example 9

### Cell Markers in Postpartum-Derived Cells

In the preceding example, similarities and differences in cells derived from the human placenta and the human umbilical cord were assessed by comparing their gene expression profiles with those of cells derived from other sources (using an oligonucleotide array). Six “signature” genes were identified: oxidized LDL receptor 1, interleukin-8, rennin, reticulon, chemokine receptor ligand 3 (CXC ligand 3), and granulocyte chemotactic protein 2 (GCP-2). These “signature” genes were expressed at relatively high levels in postpartum-derived cells.

The procedures described in this example were conducted to verify the microarray data and find concordance/discordance between gene and protein expression, as well as to establish a series of reliable assay for detection of unique identifiers for placenta- and umbilicus-derived cells.

### Methods & Materials

Cells:

Placenta-derived cells (three isolates, including one isolate predominately neonatal as identified by karyotyping analysis), umbilicus-derived cells (four isolates), and Normal Human Dermal Fibroblasts (NHDF; neonatal and adult) grown in Growth Medium with penicillin/streptomycin in a gelatin-coated T75 flask. Mesechymal Stem Cells (MSCS) were grown in Mesenchymal Stem Cell Growth Medium Bullet kit (MSCGM; Cambrex, Walkerville, Md.).

For the IL-8 protocol, cells were thawed from liquid nitrogen and plated in gelatin-coated flasks at 5,000 cells/cm², grown for 48 hours in Growth Medium and then grown for further 8 hours in 10 milliliters of serum starvation medium [DMEM--low glucose (Gibco, Carlsbad, Calif.), penicillin/streptomycin (Gibco, Carlsbad, Calif.) and 0.1% (w/v) Bovine Serum Albumin (BSA; Sigma, St. Louis, Mo.)]. After this treatment RNA was extracted and the supernatants were centrifuged at 150×g for 5 minutes to remove cellular debris. Supernatants were then frozen at −80° C. for ELISA analysis.

Cell Culture for ELISA Assay:

Postpartum cells derived from placenta and umbilicus, as well as human fibroblasts derived from human neonatal foreskin were cultured in Growth Medium in gelatin-coated T75 flasks. Cells were frozen at passage 11 in liquid nitrogen. Cells were thawed and transferred to 15-milliliter centrifuge tubes. After centrifugation at 150×g for 5 minutes, the supernatant was discarded. Cells were resuspended in 4 milliliters culture medium and counted. Cells were grown in a 75 cm² flask containing 15 milliliters of Growth Medium at 375,000 cells/flask for 24 hours. The medium was changed to a serum starvation medium for 8 hours. Serum starvation medium was collected at the end of incubation, centrifuged at 14,000×g for 5 minutes (and stored at −20° C.).

To estimate the number of cells in each flask, 2 milliliters of tyrpsin/EDTA (Gibco, Carlsbad, Calif.) was added each flask. After cells detached from the flask, trypsin activity was neutralized with 8 milliliters of Growth Medium. Cells were transferred to a 15 milliliters centrifuge tube and centrifuged at 150×g for 5 minutes. Supernatant was removed and 1 milliliter Growth Medium was added to each tube to resuspend the cells. Cell number was estimated using a hemocytometer.

ELISA Assay:

The amount of IL-8 secreted by the cells into serum starvation medium was analyzed using ELISA assays (R&D Systems, Minneapolis, Minn.). All assays were tested according to the instructions provided by the manufacturer.

Total RNA Isolation:

RNA was extracted from confluent postpartum-derived cells and fibroblasts or for IL-8 expression from cells treated as described above. Cells were lysed with 350 microliters buffer RLT containing beta-mercaptoethanol (Sigma, St. Louis, Mo.) according to the manufacturer's instructions (RNeasy® Mini Kit; Qiagen, Valencia, Calif.). RNA was extracted according to the manufacturer's instructions (RNeasy® Mini Kit; Qiagen, Valencia, Calif.) and subjected to DNase treatment (2.7 U/sample) (Sigma St. Louis, Mo.). RNA was eluted with 50 microliters DEPC-treated water and stored at −80° C.

Reverse Transcription:

RNA was also extracted from human placenta and umbilicus. Tissue (30 milligram) was suspended in 700 microliters of buffer RLT containing 2-mercaptoethanol. Samples were mechanically homogenized and the RNA extraction proceeded according to manufacturer's specification. RNA was extracted with 50 microliters of DEPC-treated water and stored at −80° C. RNA was reversed transcribed using random hexamers with the TagMan® reverse transcription reagents (Applied Biosystems, Foster City, Calif.) at 25° C. for 10 minutes, 37° C. for 60 minutes, and 95° C. for 10 minutes. Samples were stored at −20° C.

Genes identified by cDNA microarray as uniquely regulated in postpartum cells (signature genes--including oxidized LDL receptor, interleukin-8, rennin and reticulon), were further investigated using real-time and conventional PCR.

Real-Time PCR:

PCR was performed on cDNA samples using Assays-on-Demand® gene expression products: oxidized LDL receptor (Hs00234028); rennin (Hs00166915); reticulon (Hs003825 15); CXC ligand 3 (Hs00171061); GCP-2 (Hs00605742); IL-8 (Hs00174103); and GAPDH (Applied Biosystems, Foster City, Calif.) were mixed with cDNA and TagMan® Universal PCR master mix according to the manufacturer's instructions (Applied Biosystems, Foster City, Calif.) using a 7000 sequence detection system with ABI Prism 7000 SDS software (Applied Biosystems, Foster City, Calif.). Thermal cycle conditions were initially 50° C. for 2 min and 95° C. for 10 min, followed by 40 cycles of 95° C. for 15 sec and 60° C. for 1 min. PCR data was analyzed according to manufacturer's specifications (User Bulletin #2 from Applied Biosystems for ABI Prism 7700 Sequence Detection System).

Conventional PCR:

Conventional PCR was performed using an ABI PRISM 7700 (Perkin Elmer Applied Biosystems, Boston, Mass., USA) to confirm the results from real-time PCR. PCR was performed using 2 microliters of cDNA solution, 1× AmpliTaq Gold universal mix PCR reaction buffer (Applied Biosystems, Foster City, Calif.) and initial denaturation at 94° C. for 5 minutes. Amplification was optimized for each primer set. For IL-8, CXC ligand 3, and reticulon (94° C. for 15 seconds, 55° C. for 15 seconds and 72° C. for 30 seconds for 30 cycles); for rennin (94° C. for 15 seconds, 53° C. for 15 seconds and 72° C. for 30 seconds for 38 cycles); for oxidized LDL receptor and GAPDH (94° C. for 15 seconds, 55° C. for 15 seconds and 72° C. for 30 seconds for 33 cycles). Primers used for amplification are listed in Table 9-1. Primer concentration in the final PCR reaction was 1 micromolar except for GAPDH, which was 0.5 micromolar. GAPDH primers were the same as real-time PCR, except that the manufacturer's TaqMan® probe was not added to the final PCR reaction. Samples were run on 2% (w/v) agarose gel and stained with ethidium bromide (Sigma, St. Louis, Mo.). Images were captured using a 667 Universal Twinpack film (VWR International, South Plainfield, N.J.) using a focal length Polaroid camera (VWR International, South Plainfield, N.J.).

TABLE 9-1
Primers used

| Primer name | Primers |
|---|---|
| Oxidized LDL | S: 5′-GAGAAATCCAAAGAGCAAATGG-3 |
| receptor | (SEQ ID NO: 1) |
| | A: 5′-AGAATGGAAAACTGGAATAGG-3′ |
| | (SEQ ID NO: 2) |
| Renin | S: 5′-TCTTCGATGCTTCGGATTCC-3′ |
| | (SEQ ID NO: 3) |
| | A: 5′-GAATTCTCGGAATCTCTGTTG-3′ |
| | (SEQ ID NO: 4) |
| Reticulon | S: 5′-TTACAAGCAGTGCAGAAAACC-3′ |
| | (SEQ ID NO: 5) |
| | A: 5′-AGTAAACATTGAAACCACAGCC-3′ |
| | (SEQ ID NO: 6) |
| Interleukin-8 | S: 5′-TCTGCAGCTCTGTGTGAAGG-3′ |
| | (SEQ ID NO: 7) |
| | A: 5′-CTTCAAAAACTTCTCCACAACC-3′ |
| | (SEQ ID NO: 8) |
| Chemokine (CXC) | S: 5′-CCCACGCCACGCTCTCC-3′ |
| ligand 3 | (SEQ ID NO: 9) |
| | A: 5′-TCCTGTCAGTTGGTGCTCC-3′ |
| | (SEQ ID NO: 10) |

Immunofluorescence:

PPDCs were fixed with cold 4% (w/v) paraformaldehyde (Sigma-Aldrich, St. Louis, Mo.) for 10 minutes at room temperature. One isolate each of umbilicus- and placenta-derived cells at passage 0 (PO) (directly after isolation) and passage 11 (P 11) (two isolates of placenta-derived, two isolates of umbilicus-derived cells) and fibroblasts (P 11) were used Immunocytochemistry was performed using antibodies directed against the following epitopes: vimentin (1:500, Sigma, St. Louis, Mo.), desmin (1:150; Sigma—raised against rabbit; or 1:300; Chemicon, Temecula, Calif.—raised against mouse,), alpha-smooth muscle actin (SMA; 1:400; Sigma), cytokeratin 18 (CK18; 1:400; Sigma), von Willebrand Factor (vWF; 1:200; Sigma), and CD34 (human CD34 Class III; 1:100; DAKOCytomation, Carpinteria, Calif.). In addition, the following markers were tested on passage 11 postpartum cells: anti-human GRO alpha--PE (1:100; Becton Dickinson, Franklin Lakes, N.J.), anti-human GCP-2 (1:100; Santa Cruz Biotech, Santa Cruz, Calif.), anti-human oxidized LDL receptor 1 (ox-LDL R1; 1:100; Santa Cruz Biotech), and anti-human NOGA-A (1:100; Santa Cruz, Biotech).

Cultures were washed with phosphate-buffered saline (PBS) and exposed to a protein blocking solution containing PBS, 4% (v/v) goat serum (Chemic on, Temecula, Calif.), and 0.3% (v/v) Triton (Triton X-100; Sigma, St. Louis, Mo.) for 30 minutes to access intracellular antigens. Where the epitope of interest was located on the cell surface (CD34, ox-LDL R1), Triton X-100 was omitted in all steps of the procedure in order to prevent epitope loss. Furthermore, in instances where the primary antibody was raised against goat (GCP-2, ox-LDL R1, NOGO-A), 3% (v/v) donkey serum was used in place of goat serum throughout. Primary antibodies, diluted in blocking solution, were then applied to the cultures for a period of 1 hour at room temperature. The primary antibody solutions were removed and the cultures were washed with PBS prior to application of secondary antibody solutions (1 hour at room temperature) containing block along with goat anti-mouse IgG--Texas Red (1:250; Molecular Probes, Eugene, Oreg.) and/or goat anti-rabbit IgG--Alexa 488 (1:250; Molecular Probes) or donkey anti-goat IgG--FITC (1:150, Santa Cruz Biotech). Cultures were then washed and 10 micromolar DAPI (Molecular Probes) applied for 10 minutes to visualize cell nuclei.

Following immunostaining, fluorescence was visualized using an appropriate fluorescence filter on an Olympus® inverted epi-fluorescent microscope (Olympus, Melville, N.Y.). In all cases, positive staining represented fluorescence signal above control staining where the entire procedure outlined above was followed with the exception of application of a primary antibody solution. Representative images were captured using a digital color video camera and ImagePro® software (Media Cybernetics, Carlsbad, Calif.). For triple-stained samples, each image was taken using only one emission filter at a time. Layered montages were then prepared using Adobe Photoshop® software (Adobe, San Jose, Calif.).

Preparation of Cells for FACS Analysis:

Adherent cells in flasks were washed in phosphate buffered saline (PBS) (Gibco, Carlsbad, Calif.) and detached with Trypsin/EDTA (Gibco, Carlsbad, Calif.). Cells were harvested, centrifuged, and re-suspended 3% (v/v) FBS in PBS at a cell concentration of 1×10 7 per milliliter. One hundred microliter aliquots were delivered to conical tubes. Cells stained for intracellular antigens were permeabilized with Perm/Wash buffer (BD Pharmingen, San Diego, Calif.). Antibody was added to aliquots as per manufactures specifications and the cells were incubated for in the dark for 30 minutes at 4° C. After incubation, cells were washed with PBS and centrifuged to remove excess antibody. Cells requiring a secondary antibody were resuspended in 100 microliters of 3% FBS. Secondary antibody was added as per manufactures specification and the cells were incubated in the dark for 30 minutes at 4° C. After incubation, cells were washed with PBS and centrifuged to remove excess secondary antibody. Washed cells were resuspended in 0.5 milliliters PBS and analyzed by flow cytometry. The following antibodies were used: oxidized LDL receptor 1 (sc-5813; Santa Cruz, Biotech), GROa (555042; BD Pharmingen, Bedford, Mass.), Mouse IgG1 kappa, (P-4685 and M-5284; Sigma), Donkey against Goat IgG (sc-3743; Santa Cruz, Biotech.). Flow cytometry analysis was performed with FACScalibur™ (Becton Dickinson San Jose, Calif.).

### Results

Results of real-time PCR for selected “signature” genes performed on cDNA from cells derived from human placentae, adult and neonatal fibroblasts and Mesenchymal Stem Cells (MSCs) indicate that both oxidized LDL receptor and rennin were expressed at higher level in the placenta-derived cells as compared to other cells. The data obtained from real-time PCR were analyzed by the AACT method and expressed on a logarithmic scale. Levels of reticulon and oxidized LDL receptor expression were higher in umbilicus-derived cells as compared to other cells. No significant difference in the expression levels of CXC ligand 3 and GCP-2 were found between postpartum-derived cells and controls. The results of real-time PCR were confirmed by conventional PCR. Sequencing of PCR products further validated these observations. No significant difference in the expression level of CXC ligand 3 was found between postpartum-derived cells and controls using conventional PCR CXC ligand 3 primers listed above in Table 9-1.

The production of the cytokine, IL-8 in postpartum was elevated in both Growth Medium-cultured and serum-starved postpartum-derived cells. All real-time PCR data was validated with conventional PCR and by sequencing PCR products.

When supernatants of cells grown in serum-free medium were examined for the presence of IL-8, the highest amounts were detected in media derived from umbilical cells and some isolates of placenta cells (Table 9-2). No IL-8 was detected in medium derived from human dermal fibroblasts.

TABLE 9-2
IL-8 protein expression measured by ELISA

| Cell type | IL-8 |
|---|---|
| Human fibroblasts | ND |
| Placenta Isolate 1 | ND |
| UMBC Isolate 1 | 2058.42 ± 144.67 |
| Placenta Isolate 2 | ND |
| UMBC Isolate 2 | 2368.86 ± 22.73 |
| Placenta Isolate 3 (normal O₂) | 17.27 ± 8.63 |
| Placenta Isolate 3 (low O₂, | 264.92 ± 9.88 |
| W/O BME) | |

Results of the ELISA assay for interleukin-8 (IL-8) performed on placenta-and umbilical cord-derived cells as well as human skin fibroblasts. Values are presented here are picogram/million cells, n = 2, sem.
ND: Not Detected

Placenta-derived cells were also examined for the production of oxidized LDL receptor, GCP-2 and GROalpha by FACS analysis. Cells tested positive for GCP-2. Oxidized LDL receptor and GRO were not detected by this method.

Placenta-derived cells were also tested for the production of selected proteins by immunocytochemical analysis Immediately after isolation (passage 0), cells derived from the human placenta were fixed with 4% paraformaldehyde and exposed to antibodies for six proteins: von Willebrand Factor, CD34, cytokeratin 18, desmin, alpha-smooth muscle actin, and vimentin. Cells stained positive for both alpha-smooth muscle actin and vimentin. This pattern was preserved through passage 11. Only a few cells (<5%) at passage 0 stained positive for cytokeratin 18.

Cells derived from the human umbilical cord at passage 0 were probed for the production of selected proteins by immunocytochemical analysis Immediately after isolation (passage 0), cells were fixed with 4% paraformaldehyde and exposed to antibodies for six proteins: von Willebrand Factor, CD34, cytokeratin 18, desmin, alpha-smooth muscle actin, and vimentin. Umbilicus-derived cells were positive for alpha-smooth muscle actin and vimentin, with the staining pattern consistent through passage 11.

Summary:

Concordance between gene expression levels measured by microarray and PCR (both real-time and conventional) has been established for four genes: oxidized LDL receptor 1, rennin, reticulon, and IL-8. The expression of these genes was differentially regulated at the mRNA level in PPDCs, with IL-8 also differentially regulated at the protein level. The presence of oxidized LDL receptor was not detected at the protein level by FACS analysis in cells derived from the placenta. Differential expression of GCP-2 and CXC ligand 3 was not confirmed at the mRNA level, however GCP-2 was detected at the protein level by FACS analysis in the placenta-derived cells. Although this result is not reflected by data originally obtained from the micro array experiment, this may be due to a difference in the sensitivity of the methodologies.

Immediately after isolation (passage 0), cells derived from the human placenta stained positive for both alpha-smooth muscle actin and vimentin. This pattern was also observed in cells at passage 11. Vimentin and alpha-smooth muscle actin expression may be preserved in cells with passaging, in the Growth Medium and under the conditions utilized in these procedures. Cells derived from the human umbilical cord at passage 0 were probed for the expression of alpha-smooth muscle actin and vimentin, and were positive for both. The staining pattern was preserved through passage 11.

### Example 10

### In Vitro Immunological Evaluation of Postpartum-Derived Cells

Postpartum-derived cells (PPDCs) were evaluated in vitro for their immunological characteristics in an effort to predict the immunological response, if any, these cells would elicit upon in vivo transplantation. PPDCs were assayed by flow cytometry for the presence of HLA-DR, HLA-DP, HLA-DQ, CD80, CD86, and B7-H2. These proteins are expressed by antigen-presenting cells (APe) and are required for the direct stimulation of naïve CD4+T cells (Abbas & Lichtman, CELLULAR AND MOLECULAR IMMUNOLOGY, 5th Ed. (2003) Saunders, Philadelphia, p. 171). The cell lines were also analyzed by flow cytometry for the expression of HLA-G (Abbas & Lichtman, 2003, supra), CD 178 (Coumans, et al., (1999) Journal of Immunological Methods 224, 185-196), and PD-L2 (Abbas & Lichtman, 2003, supra; Brown, et. al. (2003) The Journal of Immunology, 170:1257-1266). The expression of these proteins by cells residing in placental tissues is thought to mediate the immuno-privileged status of placental tissues in utero. To predict the extent to which placenta- and umbilicus-derived cell lines elicit an immune response in vivo, the cell lines were tested in a one-way mixed lymphocyte reaction (MLR).

### Methods & Materials

Cell Culture:

Cells were cultured to confluence in Growth Medium containing penicillin/streptomycin in T75 flasks (Corning Inc., Corning, N.Y.) coated with 2% gelatin (Sigma, St. Louis, Mo.).

Antibody Staining:

Cells were washed in phosphate buffered saline (PBS) (Gibco, Carlsbad, Calif.) and detached with Trypsin/EDTA (Gibco, Carlsbad, Mo.). Cells were harvested, centrifuged, and re-suspended in 3% (v/v) FBS in PBS at a cell concentration of 1×10⁷ per milliliter. Antibody (Table 10-1) was added to one hundred microliters of cell suspension as per manufacturer's specifications and incubated in the dark for 30 minutes at 4° C. After incubation, cells were washed with PBS and centrifuged to remove unbound antibody. Cells were re-suspended in five hundred microliters of PBS and analyzed by flow cytometry using a FACSCalibur™ instrument (Becton Dickinson, San Jose, Calif.).

TABLE 10-1
Antibodies

| | | Catalog |
|---|---|---|
| Antibody | Manufacturer | Number |
| HLA-DRDPDQ | BD Pharmingen | 555558 |
| | (San Diego, CA) | |
| CD80 | BD Pharmingen | 557227 |
| | (San Diego, CA) | |
| CD86 | BD Pharmingen | 555665 |
| | (San Diego, CA) | |
| B7-H2 | BD Pharmingen | 552502 |
| | (San Diego, CA) | |
| HLA-G | Abcam | ab 7904-100 |
| | (Cambridgeshire, UK) | |
| CD 178 | Santa Cruz (San Cruz, CA) | sc-19681 |
| PD-L2 | BD Pharmingen | 557846 |
| | (San Diego, CA) | |
| Mouse IgG2a | Sigma (St. Louis, MO) | F-6522 |
| Mouse IgG1kappa | Sigma (St. Louis, MO) | P-4685 |

Mixed Lymphocyte Reaction:

Cryopreserved vials of passage 10 umbilicus-derived cells labeled as cell line A and passage 11 placenta-derived cells labeled as cell line B were sent on dry ice to CTBR (Senneville, Quebec) to conduct a mixed lymphocyte reaction using CTBR SOP No. CAC-031. Peripheral blood mononuclear cells (PBMCs) were collected from multiple male and female volunteer donors. Stimulator (donor) allogeneic PBMC, autologous PBMC, and postpartum cell lines were treated with mitomycin C. Autologous and mitomycin C-treated stimulator cells were added to responder (recipient) PBMCs and cultured for 4 days. After incubation, [³H]-thymidine was added to each sample and cultured for 18 hours. Following harvest of the cells, radiolabeled DNA was extracted, and [³H]-thymidine incorporation was measured using a scintillation counter.

The stimulation index for the allogeneic donor (SIAD) was calculated as the mean proliferation of the receiver plus mitomycin C-treated allogeneic donor divided by the baseline proliferation of the receiver. The stimulation index of the PPDCs was calculated as the mean proliferation of the receiver plus mitomycin C-treated postpartum cell line divided by the baseline proliferation of the receiver.

### Results

Mixed Lymphocyte Reaction—Placenta-Derived Cells:

Seven human volunteer blood donors were screened to identify a single allogeneic donor that would exhibit a robust proliferation response in a mixed lymphocyte reaction with the other six blood donors. This donor was selected as the allogeneic positive control donor. The remaining six blood donors were selected as recipients. The allogeneic positive control donor and placenta-derived cell lines were treated with mitomycin C and cultured in a mixed lymphocyte reaction with the six individual allogeneic receivers. Reactions were performed in triplicate using two cell culture plates with three receivers per plate (Table 10-2). The average stimulation index ranged from 1.3 (plate 2) to 3 (plate 1) and the allogeneic donor positive controls ranged from 46.25 (plate 2) to 279 (plate 1) (Table 10-3).

TABLE 10-2
Mixed Lymphocyte Reaction Data-Cell Line B (Placenta)
DPM for Proliferation Assay
Plate ID: Plate 1

| Analytical | Culture | Replicates | | | | | |
|---|---|---|---|---|---|---|---|
| number | System | 1 | 2 | 3 | Mean | SD | CV |
| IM03-7769 | Proliferation baseline of receiver | 79 | 119 | 138 | 112.0 | 30.12 | 26.9 |
| | Control of autostimulation | 241 | 272 | 175 | 229.3 | 49.54 | 21.6 |
| | (Mitomycin C treated autologous cells) | | | | | | |
| | MLR allogenic donor IM03-7768 | 23971 | 22352 | 20921 | 22414.7 | 1525.97 | 6.8 |
| | (Mitomycin C treated) | | | | | | |
| | MLR with cell line | 664 | 559 | 1090 | 771.0 | 281.21 | 36.5 |
| | (Mitomycin C treated cell type B) | | | | | | |
| SI (donor) | | | | | 200 | | |
| SI (cell line) | | | | | 7 | | |
| IM03-7770 | Proliferation baseline of receiver | 206 | 134 | 262 | 200.7 | 64.17 | 32.0 |
| | Control of autostimulation | 1091 | 602 | 524 | 739.0 | 307.33 | 41.6 |
| | (Mitomycin C treated autologous cells) | | | | | | |
| | MLR allogenic donor IM03-7768 | 45005 | 43729 | 44071 | 44268.3 | 660.49 | 1.5 |
| | (Mitomycin C treated) | | | | | | |
| | MLR with cell line | 533 | 2582 | 2376 | 1830.3 | 1128.24 | 61.6 |
| | (Mitomycin C treated cell type B) | | | | | | |
| SI (donor) | | | | | 221 | | |
| SI (cell line) | | | | | 9 | | |
| IM03-7771 | Proliferation baseline of receiver | 157 | 87 | 128 | 124.0 | 35.17 | 28.4 |
| | Control of autostimulation | 293 | 138 | 508 | 313.0 | 185.81 | 59.4 |
| | (Mitomycin C treated autologous cells) | | | | | | |
| | MLR allogenic donor IM03-7768 | 24497 | 34348 | 31388 | 30077.7 | 5054.53 | 16.8 |
| | (Mitomycin C treated) | | | | | | |
| | MLR with cell line | 601 | 643 | a | 622.0 | 29.70 | 4.8 |
| | (Mitomycin C treated cell type B) | | | | | | |
| SI (donor) | | | | | 243 | | |
| SI (cell line) | | | | | 5 | | |
| IM03-7772 | Proliferation baseline of receiver | 56 | 98 | 51 | 68.3 | 25.81 | 37.8 |
| | Control of autostimulation | 133 | 120 | 213 | 155.3 | 50.36 | 32.4 |
| | (Mitomycin C treated autologous cells) | | | | | | |
| | MLR allogenic donor IM03-7768 | 14222 | 20076 | 22168 | 18822.0 | 4118.75 | 21.9 |
| | (Mitomycin C treated) | | | | | | |
| | MLR with cell line | a | a | a | a | a | a |
| | (Mitomycin C treated cell type B) | | | | | | |
| SI (donor) | | | | | 275 | | |
| SI (cell line) | | | | | a | | |
| IM03-7768 | Proliferation baseline of receiver | 84 | 242 | 208 | 178.0 | 83.16 | 46.7 |
| (allogenic donor) | Control of autostimulation | 361 | 617 | 304 | 427.3 | 166.71 | 39.0 |
| | (Mitomycin treated autologous cells) | | | | | | |
| Cell line type B | Proliferation baseline of receiver | 126 | 124 | 143 | 131.0 | 10.44 | 8.0 |
| | Control of autostimulation | 822 | 1075 | 487 | 794.7 | 294.95 | 37.1 |
| | (Mitomycin treated autologous cells) | | | | | | |

Plate ID: Plate 2

| Analytical | Culture | Replicates | | | | | |
|---|---|---|---|---|---|---|---|
| number | System | 1 | 2 | 3 | Mean | SD | CV |
| IM03-7773 | Proliferation baseline of receiver | 908 | 181 | 330 | 473.0 | 384.02 | 81.2 |
| | Control of autostimulation | 269 | 405 | 572 | 415.3 | 151.76 | 36.5 |
| | (Mitomycin C treated autologous cells) | | | | | | |
| | MLR allogenic donor IM03-7768 | 29151 | 28691 | 28315 | 28719.0 | 418.70 | 1.5 |
| | (Mitomycin C treated) | | | | | | |
| | MLR with cell line | 567 | 732 | 905 | 734.7 | 169.02 | 23.0 |
| | (Mitomycin C treated cell type B) | | | | | | |
| SI (donor) | | | | | 61 | | |
| SI (cell line) | | | | | 2 | | |
| IM03-7774 | Proliferation baseline of receiver | 893 | 1376 | 185 | 818.0 | 599.03 | 73.2 |
| | Control of autostimulation | 261 | 381 | 568 | 403.3 | 154.71 | 38.4 |
| | (Mitomycin C treated autologous cells) | | | | | | |
| | MLR allogenic donor IM03-7768 | 53101 | 42839 | 48283 | 48074.3 | 5134.18 | 10.7 |
| | (Mitomycin C treated) | | | | | | |
| | MLR with cell line | 515 | 789 | 294 | 532.7 | 247.97 | 46.6 |
| | (Mitomycin C treated cell type B) | | | | | | |
| SI (donor) | | | | | 59 | | |
| SI (cell line) | | | | | 1 | | |
| IM03-7775 | Proliferation baseline of receiver | 1272 | 300 | 544 | 705.3 | 505.69 | 71.7 |
| | Control of autostimulation | 232 | 199 | 484 | 305.0 | 155.89 | 51.1 |
| | (Mitomycin C treated autologous cells) | | | | | | |
| | MLR allogenic donor IM03-7768 | 23554 | 10523 | 28965 | 21014.0 | 9479.74 | 45.1 |
| | (Mitomycin C treated) | | | | | | |
| | MLR with cell line | 768 | 924 | 563 | 751.7 | 181.05 | 24.1 |
| | (Mitomycin C treated cell type B) | | | | | | |
| SI (donor) | | | | | 30 | | |
| SI (cell line) | | | | | 1 | | |
| IM03-7776 | Proliferation baseline of receiver | 1530 | 137 | 1046 | 904.3 | 707.22 | 78.2 |
| | Control of autostimulation | 420 | 218 | 394 | 344.0 | 109.89 | 31.9 |
| | (Mitomycin C treated autologous cells) | | | | | | |
| | MLR allogenic donor IM03-7768 | 28893 | 32493 | 34746 | 32044.0 | 2952.22 | 9.2 |
| | (Mitomycin C treated) | | | | | | |
| | MLR with cell line | a | a | a | a | a | a |
| | (Mitomycin C treated cell type B) | | | | | | |
| SI (donor) | | | | | 35 | | |
| SI (cell line) | | | | | a | | |

TABLE 10-3
Average stimulation index of placenta cells and
an allogeneic donor in a mixed lymphocyte
reaction with six individual allogeneic receivers.

| | Recipient | Placenta |
|---|---|---|
| Plate 1 (receivers 1-3) | 279 | 3 |
| Plate 2 (receivers 4-6) | 46.25 | 1.3 |

Mixed Lymphocyte Reaction—Umbilicus-Derived Cells:

Six human volunteer blood donors were screened to identify a single allogeneic donor that will exhibit a robust proliferation response in a mixed lymphocyte reaction with the other five blood donors. This donor was selected as the allogeneic positive control donor. The remaining five blood donors were selected as recipients. The allogeneic positive control donor and placenta cell lines were mitomycin C-treated and cultured in a mixed lymphocyte reaction with the five individual allogeneic receivers. Reactions were performed in triplicate using two cell culture plates with three receivers per plate (Table 10-4). The average stimulation index ranged from 6.5 (plate 1) to 9 (plate 2) and the allogeneic donor positive controls ranged from 42.75 (plate 1) to 70 (plate 2) (Table 10-5).

TABLE 10-4
Mixed Lymphocyte Reaction Data-Cell Line A (Umbilical cord)
DPM for Proliferation Assay
Plate ID: Plate 1

| Analytical | Culture | Replicates | | | | | |
|---|---|---|---|---|---|---|---|
| number | System | 1 | 2 | 3 | Mean | SD | CV |
| IM04-2478 | Proliferation baseline of receiver | 1074 | 406 | 391 | 623.7 | 390.07 | 62.5 |
| | Control of autostimulation | 672 | 510 | 1402 | 861.3 | 475.19 | 55.2 |
| | (Mitomycin C treated autologous cells) | | | | | | |
| | MLR allogenic donor IM04-2477 | 43777 | 48391 | 38231 | 43466.3 | 5087.12 | 11.7 |
| | (Mitomycin C treated) | | | | | | |
| | MLR with cell line | 2914 | 5622 | 6109 | 4881.7 | 1721.36 | 35.3 |
| | (Mitomycin C treated cell type A) | | | | | | |
| SI (donor) | | | | | 70 | | |
| SI (cell line) | | | | | 8 | | |
| IM04-2479 | Proliferation baseline of receiver | 530 | 508 | 527 | 521.7 | 11.93 | 2.3 |
| | Control of autostimulation | 701 | 567 | 1111 | 793.0 | 283.43 | 35.7 |
| | (Mitomycin C treated autologous cells) | | | | | | |
| | MLR allogenic donor IM04-2477 | 25593 | 24732 | 22707 | 24344.0 | 1481.61 | 6.1 |
| | (Mitomycin C treated) | | | | | | |
| | MLR with cell line | 5086 | 3932 | 1497 | 3505.0 | 1832.21 | 52.3 |
| | (Mitomycin C treated cell type A) | | | | | | |
| SI (donor) | | | | | 47 | | |
| SI (cell line) | | | | | 7 | | |
| IM04-2480 | Proliferation baseline of receiver | 1192 | 854 | 1330 | 1125.3 | 244.90 | 21.8 |
| | Control of autostimulation | 2963 | 993 | 2197 | 2051.0 | 993.08 | 48.4 |
| | (Mitomycin C treated autologous cells) | | | | | | |
| | MLR allogenic donor IM04-2477 | 25416 | 29721 | 23757 | 26298.0 | 3078.27 | 11.7 |
| | (Mitomycin C treated) | | | | | | |
| | MLR with cell line | 2596 | 5076 | 3426 | 3699.3 | 1262.39 | 34.1 |
| | (Mitomycin C treated cell type A) | | | | | | |
| SI (donor) | | | | | 23 | | |
| SI (cell line) | | | | | 3 | | |
| IM04-2481 | Proliferation baseline of receiver | 695 | 451 | 555 | 567.0 | 122.44 | 21.6 |
| | Control of autostimulation | 738 | 1252 | 464 | 818.0 | 400.04 | 48.9 |
| | (Mitomycin C treated autologous cells) | | | | | | |
| | MLR allogenic donor IM04-2477 | 13177 | 24885 | 15444 | 17835.3 | 6209.52 | 34.8 |
| | (Mitomycin C treated) | | | | | | |
| | MLR with cell line | 4495 | 3671 | 4674 | 4280.0 | 534.95 | 12.5 |
| | (Mitomycin C treated cell type A) | | | | | | |
| SI (donor) | | | | | 31 | | |
| SI (cell line) | | | | | 8 | | |

Plate ID: Plate 2

| Analytical | Culture | Replicates | | | | | |
|---|---|---|---|---|---|---|---|
| number | System | 1 | 2 | 3 | Mean | SD | CV |
| IM04-2482 | Proliferation baseline of receiver | 432 | 533 | 274 | 413.0 | 130.54 | 31.6 |
| | Control of autostimulation | 1459 | 633 | 598 | 896.7 | 487.31 | 54.3 |
| | (Mitomycin C treated autologous cells) | | | | | | |
| | MLR allogenic donor IM04-2477 | 24286 | 30823 | 31346 | 28818.3 | 3933.82 | 13.7 |
| | (Mitomycin C treated) | | | | | | |
| | MLR with cell line | 2762 | 1502 | 6723 | 3662.3 | 2724.46 | 74.4 |
| | (Mitomycin C treated cell type A) | | | | | | |
| SI (donor) | | | | | 70 | | |
| SI (cell line) | | | | | 9 | | |
| IM04-2477 | Proliferation baseline of receiver | 312 | 419 | 349 | 360.0 | 54.34 | 15.1 |
| (allogenic donor) | Control of autostimulation | 567 | 604 | 374 | 515.0 | 123.50 | 24.0 |
| | (Mitomycin treated autologous cells) | | | | | | |
| Cell line type A | Proliferation baseline of receiver | 5101 | 3735 | 2973 | 3936.3 | 1078.19 | 27.4 |
| | Control of autostimulation | 1924 | 4570 | 2153 | 2882.3 | 1466.04 | 50.9 |
| | (Mitomycin treated autologous cells) | | | | | | |

TABLE 10-5
Average stimulation index of umbilical cord-derived cells
and an allogeneic donor in a mixed lymphocyte reaction
with five individual allogeneic receivers.

| | | Umbilical |
|---|---|---|
| | Recipient | Cord |
| Plate 1 (receivers 1-4) | 42.75 | 6.5 |
| Plate 2 (receiver 5) | 70 | 9 |

Antigen Presenting Cell Markers—Placenta-Derived Cells:

Histograms of placenta-derived cells analyzed by flow cytometry show negative expression of HLA-DR, DP, DQ, CD80, CD86, and B7-H2, as noted by fluorescence value consistent with the IgG control, indicating that placental cell lines lack the cell surface molecules required to directly stimulate CD4+T cells.

Immunomodulating Markers—Placenta-Derived Cells:

Histograms of placenta-derived cells analyzed by flow cytometry show positive expression of PD-L2, as noted by the increased value of fluorescence relative to the IgG control, and negative expression of CD178 and HLA-G, as noted by fluorescence value consistent with the IgG control.

Antigen Presenting Cell Markers—Umbilicus-Derived Cells:

Histograms of umbilicus-derived cells analyzed by flow cytometry show negative expression of HLA-DR, DP, DQ, CD80, CD86, and B7-H2, as noted by fluorescence value consistent with the IgG control, indicating that umbilical cell lines lack the cell surface molecules required to directly stimulate CD4+T cells.

Immunomodulating Cell Markers—Umbilicus-Derived Cells:

Histograms of umbilicus-derived cells analyzed by flow cytometry show positive expression of PD-L2, as noted by the increased value of fluorescence relative to the IgG control, and negative expression of CD178 and HLA-G, as noted by fluorescence value consistent with the IgG control.

Summary:

In the mixed lymphocyte reactions conducted with placenta-derived cell lines, the average stimulation index ranged from 1.3 to 3, and that of the allogeneic positive controls ranged from 46.25 to 279. In the mixed lymphocyte reactions conducted with umbilicus-derived cell lines the average stimulation index ranged from 6.5 to 9, and that of the allogeneic positive controls ranged from 42.75 to 70. Placenta- and umbilicus-derived cell lines were negative for the expression of the stimulating proteins HLA-DR, HLA-DP, HLA-DQ, CD80, CD86, and B7-H2, as measured by flow cytometry. Placenta- and umbilicus-derived cell lines were negative for the expression of immuno-modulating proteins HLA-G and CD178 and positive for the expression of PD-L2, as measured by flow cytometry. Allogeneic donor PBMCs contain antigen-presenting cells expressing HLA-DR, DQ, CD8, CD86, and B 7-H2, thereby allowing for the stimulation of naïve CD4+T cells. The absence of antigen-presenting cell surface molecules on placenta- and umbilicus-derived cells required for the direct stimulation of naïve CD4+T cells and the presence of PD-L2, an immunomodulating protein, may account for the low stimulation index exhibited by these cells in a MLR as compared to allogeneic controls.

### Example 11

### Secretion of Trophic Factors by Postpartum-Derived Cells

The secretion of selected trophic factors from placenta- and umbilicus-derived cells was measured. Factors selected for detection included: (1) those known to have angiogenic activity, such as hepatocyte growth factor (HGF) (Rosen et al. (1997) Ciba Found. Symp. 212:215-26), monocyte chemotactic protein 1 (MCP-1) (Salcedo et al. (2000) Blood 96; 34-40), interleukin-8 (IL-8) (Li et al. (2003) J. Immunol. 170:3369-76), keratinocyte growth factor (KGF), basic fibroblast growth factor (bFGF), vascular endothelial growth factor (VEGF) (Hughes et al. (2004) Ann. Thorac. Surg. 77:812-8), matrix metalloproteinase 1 (TIMP1), angiopoietin 2 (ANG2), platelet derived growth factor (PDGF-bb), thrombopoietin (TPO), heparin-binding epidermal growth factor (HB-EGF), stromal-derived factor lalpha (SDF-lalpha); (2) those known to have neurotrophic/neuroprotective activity, such as brain-derived neurotrophic factor (BDNF) (Cheng et al. (2003) Dev. Biol. 258; 319-33), interleukin-6 (IL-6), granulocyte chemotactic protein-2 (GCP-2), transforming growth factor beta2 (TGFbeta2); and (3) those known to have chemokine activity, such as macrophage inflammatory protein 1 alpha (MIP1a), macrophage inflammatory protein 1 beta (MIP1b), monocyte chemoattractant-1 (MCP-1), Rantes (regulated on activation, normal T cell expressed and secreted), 1309, thymus and activation-regulated chemokine (TARe), Eotaxin, macrophage-derived chemokine (MDC), IL-8).

### Methods & Materials

Cell Culture:

PPDCs from placenta and umbilicus as well as human fibroblasts derived from human neonatal foreskin were cultured in Growth Medium with penicillin/streptomycin on gelatin-coated T75 flasks. Cells were cryopreserved at passage 11 and stored in liquid nitrogen. After thawing of the cells, Growth Medium was added to the cells followed by transfer to a 15 milliliter centrifuge tube and centrifugation of the cells at 150×g for 5 minutes. The supernatant was discarded. The cell pellet was resuspended in 4 milliliters Growth Medium, and cells were counted. Cells were seeded at 375,000 cells/75 cm² flask containing 15 milliliters of Growth Medium and cultured for 24 hours. The medium was changed to a serum-free medium (DMEM-low glucose (Gibco), 0.1% (w/v) bovine serum albumin (Sigma), penicillin/streptomycin (Gibco)) for 8 hours. Conditioned serum-free medium was collected at the end of incubation by centrifugation at 14,000×g for 5 minutes and stored at −20° C.

To estimate the number of cells in each flask, cells were washed with PBS and detached using 2 milliliters trypsin/EDTA. Trypsin activity was inhibited by addition of 8 milliliters Growth Medium. Cells were centrifuged at 150×g for 5 minutes. Supernatant was removed, and cells were resuspended in 1 milliliter Growth Medium. Cell number was estimated using a hemocytometer.

ELISA Assay:

Cells were grown at 37° C. in 5% carbon dioxide and atmospheric oxygen. Placenta-derived cells (batch 101503) also were grown in 5% oxygen or beta-mercaptoethanol (BME). The amount of MCP-1, IL-6, VEGF, SDF-lalpha, GCP-2, IL-8, and TGF-beta 2 produced by each cell sample was measured by an ELISA assay (R&D Systems, Minneapolis, Minn.). All assays were performed according to the manufacturer's instructions.

SearchLight™ Multiplexed ELISA Assay:

Chemokines (MIP1a, MIP1b, MCP-1, Rantes, 1309, TARC, Eotaxin, MDC, IL8), BDNF, and angiogenic factors (HGF, KGF, bFGF, VEGF, TIMP1, ANG2, PDGF-bb, TPO, HB-EGF were measured using SearchLight™ Proteome Arrays (Pierce Biotechnology Inc.). The Proteome Arrays are multiplexed sandwich ELISAs for the quantitative measurement of two to 16 proteins per well. The arrays are produced by spotting a 2×2, 3×3, or 4×4 pattern of four to 16 different capture antibodies into each well of a 96-well plate. Following a sandwich ELISA procedure, the entire plate is imaged to capture chemiluminescent signal generated at each spot within each well of the plate. The amount of signal generated in each spot is proportional to the amount of target protein in the original standard or sample.

### Results

ELISA Assay:

MCP-1 and IL-6 were secreted by placenta- and umbilicus-derived cells and dermal fibroblasts (Table 11-1). SDF-lalpha was secreted by placenta-derived cells cultured in 5% 0 2 and by fibroblasts. GCP-2 and IL-8 were secreted by umbilicus-derived cells and by placenta-derived cells cultured in the presence of BME or 5% 02. GCP-2 also was secreted by human fibroblasts. TGF-beta2 was not detectable by ELISA assay.

TABLE 11-1
ELISA Results: Detection of Trophic Factors

| | | | | | | | TGF- |
|---|---|---|---|---|---|---|---|
| | MCP-1 | IL-6 | VEGF | SDF-1α | GCP-2 | IL-8 | β2 |
| Fibroblast | 17 ± 1 | 61 ± 3 | 29 ± 2 | 19 ± 1 | 21 ± 1 | ND | ND |
| Placenta (042303) | 60 ± 3 | 41 ± 2 | ND | ND | ND | ND | ND |
| Umbilical (022803) | 1150 ± 74 | 4234 ± 289 | ND | ND | 160 ± 11 | 2058 ± 145 | ND |
| Placenta (071003) | 125 ± 16 | 10 ± 1 | ND | ND | ND | ND | ND |
| Umbilical (071003) | 2794 ± 84 | 1356 ± 43 | ND | ND | 2184 ± 98 | 2369 ± 23 | ND |
| Placenta (101503) BME | 21 ± 10 | 67 ± 3 | ND | ND | 44 ± 9 | 17 ± 9 | ND |
| Placenta (101503) | 77 ± 16 | 339 ± 21 | ND | 1149 ± 137 | 54 ± 2 | 265 ± 10 | ND |
| 5% O₂, W/O BME | | | | | | | |

Key: ND: Not Detected., =/− sem

SearchLight™ Multiplexed ELISA Assay:

TIMP1, TPO, KGF, HGF, FGF, HBEGF, BDNF, MIP1b, MCP1, RANTES, 1309, TARC, MDC, and IL-8 were secreted from umbilicus-derived cells (Tables 11-2 and 11-3). TIMP1, TPO, KGF, HGF, HBEGF, BDNF, MIP1a, MCP-1, RANTES, TARC, Eotaxin, and IL-8 were secreted from placenta-derived cells (Tables 11-2 and 11-3). No Ang2, VEGF, or PDGF-bb were detected.

TABLE 11-2
SEARCHLIGHT Multiplexed ELISA assay results

| | TIMP1 | ANG2 | PDGFbb | TPO | KGF | HGF | FGF | VEGF | HBEGF | BDNF |
|---|---|---|---|---|---|---|---|---|---|---|
| hFB | 19306.3 | ND | ND | 230.5 | 5.0 | ND | ND | 27.9 | 1.3 | ND |
| P1 | 24299.5 | ND | ND | 546.6 | 8.8 | 16.4 | ND | ND | 3.81.3 | ND |
| U1 | 57718.4 | ND | ND | 1240.0 | 5.8 | 559.3 | 148.7 | ND | 9.3 | 165.7 |
| P3 | 14176.8 | ND | ND | 568.7 | 5.2 | 10.2 | ND | ND | 1.9 | 33.6 |
| U3 | 21850.0 | ND | ND | 1134.5 | 9.0 | 195.6 | 30.8 | ND | 5.4 | 388.6 |

Key: hFB (human fibroblasts), P1 (placenta-derived cells (042303)), U1 (umbilicus-derived cells (022803)), P3 (placenta-derived cells(071003)), U3 (umbilicus-derived cells (071003)). ND: Not Detected.

TABLE 11-3
SEARCHLIGHT Multiplexed ELISA assay results

| | MIP1a | MIP1b | MCP1 | RANTES | I309 | TARC | Eotaxin | MDC | IL8 |
|---|---|---|---|---|---|---|---|---|---|
| hFB | ND | ND | 39.6 | ND | ND | 0.1 | ND | ND | 204.9 |
| P1 | 79.5 | ND | 228.4 | 4.1 | ND | 3.8 | 12.2 | ND | 413.5 |
| U1 | ND | 8.0 | 1694.2 | ND | 22.4 | 37.6 | ND | 18.9 | 51930.1 |
| P3 | ND | ND | 102.7 | ND | ND | 0.4 | ND | ND | 63.8 |
| U3 | ND | 5.2 | 2018.7 | 41.5 | 11.6 | 21.4 | ND | 4.8 | 10515.9 |

Key: hFB (human fibroblasts), P1 (placenta-derived PPDC (042303)), U1 (umbilicus-derived PPDC (022803)), P3 (placenta-derived PPDC (071003)), U3 (umbilicus-derived PPDC (071003)). ND: Not Detected.

### Example 12

### Short-Term Neural Differentiation of Postpartum-Derived Cells

The ability of placenta- and umbilicus-derived cells (collectively postpartum-derived cells or PPDCs) to differentiate into neural lineage cells was examined.

### Methods & Materials

Isolation and Expansion of Postpartum Cells:

PPDCs from placental and umbilical tissues were isolated and expanded as described in Example 4.

Modified Woodbury-Black Protocol (A):

This assay was adapted from an assay originally performed to test the neural induction potential of bone marrow stromal cells (1). Umbilicus-derived cells (022803) P4 and placenta-derived cells (042203) P3 were thawed and culture expanded in Growth Media at 5,000 cells/cm² until sub-confluence (75%) was reached. Cells were then trypsinized and seeded at 6,000 cells per well of a Titretek II glass slide (VWR International, Bristol, Conn.). As controls, mesenchymal stem cells (P3; 1F2155; Cambrex, Walkersville, Md.), osteoblasts (P5; CC2538; Cambrex), adipose-derived cells (Artecel, U.S. Pat. No. 6,555,374 B1) (P6; Donor 2) and neonatal human dermal fibroblasts (P6; CC2509; Cambrex) were also seeded under the same conditions.

All cells were initially expanded for 4 days in DMEM/F12 medium (Invitrogen, Carlsbad, Calif.) containing 15% (v/v) fetal bovine serum (FBS; Hyclone, Logan, Utah), basic fibroblast growth factor (bFGF; 20 nanograms/milliliter; Peprotech, Rocky Hill, N.J.), epidermal growth factor (EGF; 20 nanograms/milliliter; Peprotech) and penicillin/streptomycin (Invitrogen). After four days, cells were rinsed in phosphate-buffered saline (PBS; Invitrogen) and were subsequently cultured in DMEM/F12 medium+20% (v/v) FBS+penicillin/streptomycin for 24 hours. After 24 hours, cells were rinsed with PBS. Cells were then cultured for 1-6 hours in an induction medium which was comprised of DMEM/F12 (serum-free) containing 200 mM butylated hydroxyanisole, 10 μM potassium chloride, 5 milligram/milliliter insulin, 10 μM forskolin, 4 μM valproic acid, and 2 μM hydrocortisone (all chemicals from Sigma, St. Louis, Mo.). Cells were then fixed in 100% ice-cold methanol and immunocytochemistry was performed (see methods below) to assess human nestin protein expression.

Modified Woodbury-Black Protocol (B):

PPDCs (umbilicus (022803) P11; placenta (042203) P11 and adult human dermal fibroblasts (1F1853, P11) were thawed and culture expanded in Growth Medium at 5,000 cells/cm² until sub-confluence (75%) was reached. Cells were then trypsinized and seeded at similar density as in (A), but onto (1) 24 well tissue culture-treated plates (TCP, Falcon brand, VWR International), (2) TCP wells+2% (w/v) gelatin adsorbed for 1 hour at room temperature, or (3) TCP wells+20 μg/milliliter adsorbed mouse laminin (adsorbed for a minimum of 2 hours at 37° C.; Invitrogen).

Exactly as in (A), cells were initially expanded and media switched at the aforementioned timeframes. One set of cultures was fixed, as before, at 5 days and 6 hours, this time with ice-cold 4% (w/v) paraformaldehyde (Sigma) for 10 minutes at room temperature. In the second set of cultures, medium was removed and switched to Neural Progenitor Expansion medium (NPE) consisting of Neurobasal-A medium (Invitrogen) containing B27 (B27 supplement; Invitrogen), L-glutamine (4 mM), and penicillin/streptomycin (Invitrogen). NPE medium was further supplemented with retinoic acid (RA; 1 μM; Sigma). This medium was removed 4 days later and cultures were fixed with ice-cold 4% (w/v) paraformaldehyde (Sigma) for 10 minutes at room temperature, and stained for nestin, GFAP, and TuJ1 protein expression (see Table 12-1).

TABLE 12-1
Summary of Primary Antibodies Used

| Antibody | Concentration | Vendor |
|---|---|---|
| Rat 401 (nestin) | 1:200 | Chemicon, Temecula, CA |
| Human Nestin | 1:100 | Chemicon |
| TuJ1 (BIII Tubulin) | 1:500 | Sigma, St. Louis, MO |
| GFAP | 1:2000 | DakoCytomation, |
| | | Carpinteria, CA |
| Tyrosine hydroxylase (TH) | 1:1000 | Chemicon |
| GABA | 1:400 | Chemicon |
| Desmin (mouse) | 1:300 | Chemicon |
| alpha-alpha-smooth muscle | 1:400 | Sigma |
| actin | | |
| Human nuclear protein (hNuc) | 1:150 | Chemicon |

Two Stage Differentiation Protocol:

PPDCs (umbilicus (042203) P11, placenta (022803) P11), adult human dermal fibroblasts (P11;1F1853; Cambrex) were thawed and culture expanded in Growth Medium at 5,000 cells/cm² until sub-confluence (75%) was reached. Cells were then trypsinized and seeded at 2,000 cells/cm², but onto 24 well plates coated with laminin (BD Biosciences, Franklin Lakes, N.J.) in the presence of NPE media supplemented with bFGF (20 nanograms/milliliter; Peprotech, Rocky Hill, N.J.) and EGF (20 nanograms/milliliter; Peprotech) [whole media composition further referred to as NPE+F+E]. At the same time, adult rat neural progenitors isolated from hippocampus (P4; (062603) were also plated onto 24 welliaminin-coated plates in NPE+F+E media. All cultures were maintained in such conditions for a period of 6 days (cells were fed once during that time) at which time media was switched to the differentiation conditions listed in Table 12-2 for an additional period of 7 days. Cultures were fixed with ice-cold 4% (w/v) paraformaldehyde (Sigma) for 10 minutes at room temperature, and stained for human or rat nestin, GF AP, and TuJ1protein expression.

TABLE 12-2
Summary of Conditions for Two-Stage Differentiation Protocol

| | A | B |
|---|---|---|
| COND. # | PRE-DIFFERENTIATION | 2^{nd} STAGE DIFF |
| 1 | NPE + F (20 ng/ml) + E (20 ng/ml) | NPE + SHH (200 ng/ml) + F8 (100 ng/ml) |
| 2 | NPE + F (20 ng/ml) + E (20 ng/ml) | NPE + SHH (200 ng/ml) + F8 (100 ng/ml) + |
| | | RA (1 μM) |
| 3 | NPE + F (20 ng/ml) + E (20 ng/ml) | NPE + RA (1 μM) |
| 4 | NPE + F (20 ng/ml) + E (20 ng/ml) | NPE + F (20 ng/ml) + E (20 ng/ml) |
| 5 | NPE + F (20 ng/ml) + E (20 ng/ml) | Growth Medium |
| 6 | NPE + F (20 ng/ml) + E (20 ng/ml) | Condition 1B + MP52 (20 ng/ml) |
| 7 | NPE + F (20 ng/ml) + E (20 ng/ml) | Condition 1B + BMP7 (20 ng/ml) |
| 8 | NPE + F (20 ng/ml) + E (20 ng/ml) | Condition 1B + GDNF (20 ng/ml) |
| 9 | NPE + F (20 ng/ml) + E (20 ng/ml) | Condition 2B + MP52 (20 ng/ml) |
| 10 | NPE + F (20 ng/ml) + E (20 ng/ml) | Condition 2B + BMP7 (20 ng/ml) |
| 11 | NPE + F (20 ng/ml) + E (20 ng/ml) | Condition 2B + GDNF (20 ng/ml) |
| 12 | NPE + F (20 ng/ml) + E (20 ng/ml) | Condition 3B + MP52 (20 ng/ml) |
| 13 | NPE + F (20 ng/ml) + E (20 ng/ml) | Condition 3B + BMP7 (20 ng/ml) |
| 14 | NPE + F (20 ng/ml) + E (20 ng/ml) | Condition 3B + GDNF (20 ng/ml) |
| 15 | NPE + F (20 ng/ml) + E (20 ng/ml) | NPE + MP52 (20 ng/ml) |
| 16 | NPE + F (20 ng/ml) + E (20 ng/ml) | NPE + BMP7 (20 ng/ml) |
| 17 | NPE + F (20 ng/ml) + E (20 ng/ml) | NPE + GDNF (20 ng/ml) |

Multiple Growth Factor Protocol:

Umbilicus-derived cells (P11; (042203)) were thawed and culture expanded in Growth Medium at 5,000 cells/cm² until sub-confluence (75%) was reached. Cells were then trypsinized and seeded at 2,000 cells/cm², onto 24 welliaminin-coated plates (BD Biosciences) in the presence of NPE+F (20 nanograms/milliliter)+E (20 nanograms/milliliter). In addition, some wells contained NPE+F+E+2% FBS or 10% FBS. After four days of “pre-differentiation” conditions, all media were removed and samples were switched to NPE medium supplemented with sonic hedgehog (SHH; 200 nanograms/milliliter; Sigma, St. Louis, Mo.), FGF8 (100 nanograms/milliliter; Peprotech), BDNF (40 nanograms/milliliter; Sigma), GDNF (20 nanograms/milliliter; Sigma), and retinoic acid (1 μM; Sigma). Seven days post medium change, cultures were fixed with ice-cold 4% (w/v) paraformaldehyde (Sigma) for 10 minutes at room temperature, and stained for human nestin, GFAP, TuJ1, desmin, and alpha-smooth muscle actin expression.

Neural Progenitor Co-Culture Protocol:

Adult rat hippocampal progenitors (062603) were plated as neurospheres or single cells (10,000 cells/well) onto laminin-coated 24 well dishes (BD Biosciences) in NPE+F (20 nanograms/milliliter)+E (20 nanograms/milliliter).

Separately, umbilicus-derived cells (042203) P11 and placenta-derived cells (022803) P11 were thawed and culture expanded in NPE+F (20 nanograms/milliliter)+E (20 nanograms/milliliter) at 5,000 cells/cm² for a period of 48 hours. Cells were then trypsinized and seeded at 2,500 cells/well onto existing cultures of neural progenitors. At that time, existing medium was exchanged for fresh medium. Four days later, cultures were fixed with ice-cold 4% (w/v) paraformaldehyde (Sigma) for 10 minutes at room temperature, and stained for human nuclear protein (hNuc; Chemicon) (Table 12-1 above) to identify PPDCs.

Immunocytochemistry:

Immunocytochemistry was performed using the antibodies listed in Table 12-1. Cultures were washed with phosphate-buffered saline (PBS) and exposed to a protein blocking solution containing PBS, 4% (v/v) goat serum (Chemicon, Temecula, Calif.), and 0.3% (v/v) Triton (Triton X-100; Sigma) for 30 minutes to access intracellular antigens. Primary antibodies, diluted in blocking solution, were then applied to the cultures for a period of 1 hour at room temperature. Next, primary antibodies solutions were removed and cultures washed with PBS prior to application of secondary antibody solutions (1 hour at room temperature) containing blocking solution along with goat anti-mouse IgG--Texas Red (1:250; Molecular Probes, Eugene, Oreg.) and goat anti-rabbit IgG--Alexa 488 (1:250; Molecular Probes). Cultures were then washed and 10 micromolar DAPI (Molecular Probes) applied for 10 minutes to visualize cell nuclei.

Following immunostaining, fluorescence was visualized using the appropriate fluorescence filter on an Olympus inverted epi-fluorescent microscope (Olympus, Melville, N.Y.). In all cases, positive staining represented fluorescence signal above control staining where the entire procedure outlined above was followed with the exception of application of a primary antibody solution. Representative images were captured using a digital color video camera and ImagePro software (Media Cybernetics, Carlsbad, Calif.). For triple-stained samples, each image was taken using only one emission filter at a time. Layered montages were then prepared using Adobe Photoshop software (Adobe, San Jose, Calif.).

### Results

Modified Woodbury-Black Protocol (A):

Upon incubation in this neural induction composition, all cell types transformed into cells with bipolar morphologies and extended processes. Other larger non-bipolar morphologies were also observed. Furthermore, the induced cell populations stained positively for nestin, a marker of multipotent neural stem and progenitor cells.

Modified Woodbury-Black Protocol (B):

When repeated on tissue culture plastic (TCP) dishes, nestin expression was not observed unless laminin was pre-adsorbed to the culture surface. To further assess whether nestin-expressing cells could then go on to generate mature neurons, PPDCs and fibroblasts were exposed to NPE+RA (1 μM), a media composition known to induce the differentiation of neural stem and progenitor cells into such cells (2, 3, 4). Cells were stained for TuJ1, a marker for immature and mature neurons, GFAP, a marker of astrocytes, and nestin. Under no conditions was TuJ1 detected, nor were cells with neuronal morphology observed. Furthermore, nestin and GF AP were no longer expressed by PPDCs, as determined by immunocytochemistry.

Two-Stage Differentiation:

Umbilicus and placenta PPDC isolates (as well as human fibroblasts and rodent neural progenitors as negative and positive control cell types, respectively) were plated on laminin (neural promoting)-coated dishes and exposed to 13 different growth conditions (and two control conditions) known to promote differentiation of neural progenitors into neurons and astrocytes. In addition, two conditions were added to examine the influence of GDF5, and BMP7 on PPDC differentiation. Generally, a two-step differentiation approach was taken, where the cells were first placed in neural progenitor expansion conditions for a period of 6 days, followed by full differentiation conditions for 7 days. Morphologically, both umbilicus- and placenta-derived cells exhibited fundamental changes in cell morphology throughout the time-course of this procedure. However, neuronal or astrocytic-shaped cells were not observed except for in control, neural progenitor-plated conditions Immunocytochemistry, negative for human nestin, TuJ1, and GFAP confirmed the morphological observations.

Multiple Growth Factors:

Following one week's exposure to a variety of neural differentiation agents, cells were stained for markers indicative of neural progenitors (human nestin), neurons (TuJ1), and astrocytes (GFAP). Cells grown in the first stage in non-serum containing media had different morphologies than those cells in serum containing (2% or 10%) media, indicating potential neural differentiation. Specifically, following a two step procedure of exposing umbilicus-derived cells to EGF and bFGF, followed by SHH, FGF8, GDNF, BDNF, and retinoic acid, cells showed long extended processes similar to the morphology of cultured astrocytes. When 2% FBS or 10% FBS was included in the first stage of differentiation, cell number was increased and cell morphology was unchanged from control cultures at high density. Potential neural differentiation was not evidenced by immunocytochemical analysis for human nestin, TuJ1, or GFAP.

Neural Progenitor and PPDC Co-Culture:

PPDCs were plated onto cultures of rat neural progenitors seeded two days earlier in neural expansion conditions (NPE+F+E). While visual confirmation of plated PPDCs proved that these cells were plated as single cells, human-specific nuclear staining (hNuc) 4 days post-plating (6 days total) showed that they tended to ball up and avoid contact with the neural progenitors. Furthermore, where PPDCs attached, these cells spread out and appeared to be innervated by differentiated neurons that were of rat origin, suggesting that the PPDCs may have differentiated into muscle cells. This observation was based upon morphology under phase contrast microscopy. Another observation was that typically large cell bodies (larger than neural progenitors) possessed morphologies resembling neural progenitors, with thin processes spanning out in multiple directions. hNuc staining (found in one half of the cell's nucleus) showed that in some cases these human cells may have fused with rat progenitors and assumed their phenotype. Control wells containing only neural progenitors had fewer total progenitors and apparent differentiated cells than did co-culture wells containing umbilicus or placenta PPDCs, further indicating that both umbilicus- and placenta-derived cells influenced the differentiation and behavior of neural progenitors, either by release of chemokines and cytokines, or by contact-mediated effects.

Summary:

Multiple protocols were conducted to determine the short term potential of PPDCs to differentiate into neural lineage cells. These included phase contrast imaging of morphology in combination with immunocytochemistry for nestin, Tull, and GFAP, proteins associated with multipotent neural stem and progenitor cells, immature and mature neurons, and astrocytes, respectively.

### Example 13

### Long-Term Neural Differentiation of Postpartum-Derived Cells

The ability of umbilicus and placenta-derived cells (collectively postpartum-derived cells or PPDCs) to undergo long-term differentiation into neural lineage cells was evaluated.

### Methods & Materials

Isolation and Expansion of PPDCs:

PPDCs were isolated and expanded as described in previous Examples.

PPDC Cell Thaw and Plating:

Frozen aliquots of PPDCs (umbilicus (022803) P11; (042203) P11; (071003) P12; placenta (101503) P7) previously grown in Growth Medium were thawed and plated at 5,000 cells/cm 2 in T-75 flasks coated with laminin (BD, Franklin Lakes, N.J.) in Neurobasal-A medium (Invitrogen, Carlsbad, Calif.) containing B27 (B27 supplement, Invitrogen), L-glutamine (4 mM), and Penicillin/Streptomycin (10 milliliters), the combination of which is herein referred to as Neural Progenitor Expansion (NPE) media. NPE media was further supplemented with bFGF (20 nanograms/milliliter, Peprotech, Rocky Hill, N. J.) and EGF (20 nanograms/milliliter, Peprotech, Rocky Hill, N.J.), herein referred to as NPE+bFGF+EGF.

Control Cell Plating:

In addition, adult human dermal fibroblasts (P11, Cambrex, Walkersville, Md.) and mesenchymal stem cells (P5, Cambrex) were thawed and plated at the same cell seeding density on laminin-coated T-75 flasks in NPE+bFGF+EGF. As a further control, fibroblasts, umbilicus, and placenta PPDCs were grown in Growth Medium for the period specified for all cultures.

Cell Expansion:

Media from all cultures were replaced with fresh media once a week and cells observed for expansion. In general, each culture was passaged one time over a period of one month because of limited growth in NPE+bFGF+EGF.

Immunocytochemistry:

After a period of one month, all flasks were fixed with cold 4% (w/v) paraformaldehyde (Sigma) for 10 minutes at room temperature. Immunocytochemistry was performed using antibodies directed against TuJ1 (BIII Tubulin; 1:500; Sigma, St. Louis, Mo.) and GFAP (glial fibrillary acidic protein; 1:2000; DakoCytomation, Carpinteria, Calif.). Briefly, cultures were washed with phosphate-buffered saline (PBS) and exposed to a protein blocking solution containing PBS, 4% (v/v) goat serum (Chemic on, Temecula, Calif.), and 0.3% (v/v) Triton (Triton X-100; Sigma) for 30 minutes to access intracellular antigens. Primary antibodies, diluted in blocking solution, were then applied to the cultures for a period of 1 hour at room temperature. Next, primary antibodies solutions were removed and cultures washed with PBS prior to application of secondary antibody solutions (1 hour at room temperature) containing block along with goat anti-mouse IgG--Texas Red (1:250; Molecular Probes, Eugene, Oreg.) and goat anti-rabbit IgG--Alexa 488 (1:250; Molecular Probes). Cultures were then washed and 10 micromolar DAPI (Molecular Probes) applied for 10 minutes to visualize cell nuclei.

Following immunostaining, fluorescence was visualized using the appropriate fluorescence filter on an Olympus inverted epi-fluorescent microscope (Olympus, Melville, N.Y.). In all cases, positive staining represented fluorescence signal above control staining where the entire procedure outlined above was followed with the exception of application of a primary antibody solution. Representative images were captured using a digital color video camera and ImagePro software (Media Cybernetics, Carlsbad, Calif.). For triple-stained samples, each image was taken using only one emission filter at a time. Layered montages were then prepared using Adobe Photoshop software (Adobe, San Jose, Calif.).

TABLE 13-1
Summary of Primary Antibodies Used

| Antibody | Concentration | Vendor |
|---|---|---|
| TuJ1 (BIII Tubulin) | 1:500 | Sigma, St. Louis, MO |
| GFAP | 1:2000 | DakoCytomation, Carpinteria, CA |

### Results

NPE+bFGF+EGF media slows proliferation of PPDCs and alters their morphology Immediately following plating, a subset of PPDCs attached to the culture flasks coated with laminin. This may have been due to cell death as a function of the freeze/thaw process or because of the new growth conditions. Cells that did attach adopted morphologies different from those observed in Growth Media.

Clones of Umbilicus-Derived Cells Express Neuronal Proteins:

Cultures were fixed at one month post-thawing/plating and stained for the neuronal protein TuJ1 and GFAP, an intermediate filament found in astrocytes. While all control cultures grown in Growth Medium and human fibroblasts and MSCs grown in NPE+bFGF+EGF medium were found to be TuJ1-/GFAP-, TuJ1 was detected in the umbilicus and placenta PPDCs. Expression was observed in cells with and without neuronal-like morphologies. No expression of GFAP was observed in either culture. The percentage of cells expressing TuJ1 with neuronal-like morphologies was less than or equal to 1% of the total population (n=3 umbilicus-derived cell isolates tested). While not quantified, the percentage of TuJ1+cells without neuronal morphologies was higher in umbilicus-derived cell cultures than placenta-derived cell cultures. These results appeared specific as age-matched controls in Growth Medium did not express TuJ1.

Summary:

Methods for generating differentiated neurons (based on TuJ1 expression and neuronal morphology) from umbilicus-derived cells were developed. While expression for TuJ1 was not examined earlier than one month in vitro, it is clear that at least a small population of umbilicus-derived cells can give rise to neurons either through default differentiation or through long-term induction following one month of exposure to a minimal media supplemented with L-glutamine, basic FGF, and EGF.

### Example 14

### PPDC Trophic Factors for Neural Progenitor Support

The influence of umbilicus- and placenta-derived cells (collectively postpartum-derived cells or PPDCs) on adult neural stem and progenitor cell survival and differentiation through non-contact dependent (trophic) mechanisms was examined.

### Methods & Materials

Adult Neural Stem and Progenitor Cell Isolation:

Fisher 344 adult rats were sacrificed by CO₂ asphyxiation followed by cervical dislocation. Whole brains were removed intact using bone rongeurs and hippocampus tissue dissected based on coronal incisions posterior to the motor and somatosensory regions of the brain (Paxinos, G. & Watson, C. 1997. The Rat Brain in Stereotaxic Coordinates). Tissue was washed in Neurobasal-A medium (Invitrogen, Carlsbad, Calif.) containing B27 (B27 supplement; Invitrogen), L-glutamine (4 mM; Invitrogen), and penicillin/streptomycin (Invitrogen), the combination of which is herein referred to as Neural Progenitor Expansion (NPE) medium. NPE medium was further supplemented with bFGF (20 nanograms/milliliter, Peprotech, Rocky Hill, N.J.) and EGF (20 nanograms/milliliter, Peprotech, Rocky Hill, N.J.), herein referred to as NPE+bFGF+EGF.

Following wash, the overlying meninges were removed, and the tissue minced with a scalpel. Minced tissue was collected and trypsin/EDTA (Invitrogen) added as 75% of the total volume. DNase (100 microliters per 8 milliliters total volume, Sigma, St. Louis, Mo.) was also added. Next, the tissue/media was sequentially passed through an 18 gauge needle, 20 gauge needle, and finally a 25 gauge needle one time each (all needles from Becton Dickinson, Franklin Lakes, N.J.). The mixture was centrifuged for 3 minutes at 250 g. Supernatant was removed, fresh NPE+bFGF+EGF was added and the pellet resuspended. The resultant cell suspension was passed through a 40 micrometer cell strainer (Becton Dickinson), plated on laminin-coated T-75 flasks (Becton Dickinson) or low cluster 24-well plates (Becton Dickinson), and grown in NPE+bFGF+EGF media until sufficient cell numbers were obtained for the studies outlined.

PPDC Plating:

Postpartum-derived cells (umbilicus (022803) P12, (042103) P12, (071003) P12; placenta (042203) P12) previously grown in Growth Medium were plated at 5,000 cells/transwell insert (sized for 24 well plate) and grown for a period of one week in Growth Medium in inserts to achieve confluence.

Adult Neural Progenitor Plating:

Neural progenitors, grown as neurospheres or as single cells, were seeded onto laminin-coated 24 well plates at an approximate density of 2,000 cells/well in NPE+bFGF+EGF for a period of one day to promote cellular attachment. One day later, transwell inserts containing postpartum cells were added according to the following scheme:
a. Transwell (umbilicus-derived cells in Growth Media, 200 microliters)+neural progenitors (NPE+bFGF+EGF, 1 milliliter)
b. Transwell (placenta-derived cells in Growth Media, 200 microliters)+neural progenitors (NPE+bFGF+EGF, 1 milliliter)
c. Transwell (adult human dermal fibroblasts [1 F 1853; Cambrex, Walkersville, Md.] P12 in Growth Media, 200 microliters)+neural progenitors (NPE+bFGF+EGF, 1 milliliter)
d. Control: neural progenitors alone (NPE+bFGF+EGF, 1 milliliter)
e. Control: neural progenitors alone (NPE only, 1 milliliter)

Immunocytochemistry:

After 7 days in co-culture, all conditions were fixed with cold 4% (w/v) paraformaldehyde (Sigma) for a period of 10 minutes at room temperature. Immunocytochemistry was performed using antibodies directed against the epitopes listed in Table 14-1. Briefly, cultures were washed with phosphate-buffered saline (PBS) and exposed to a protein blocking solution containing PBS, 4% (v/v) goat serum (Chemic on, Temecula, Calif.), and 0.3% (v/v) Triton (Triton X-100; Sigma) for 30 minutes to access intracellular antigens. Primary antibodies, diluted in blocking solution, were then applied to the cultures for a period of 1 hour at room temperature. Next, primary antibodies solutions were removed and cultures washed with PBS prior to application of secondary antibody solutions (1 hour at room temperature) containing blocking solution along with goat anti-mouse IgG--Texas Red (1:250; Molecular Probes, Eugene, Oreg.) and goat anti-rabbit IgG--Alexa 488 (1:250; Molecular Probes). Cultures were then washed and 10 micromolar DAPI (Molecular Probes) applied for 10 minutes to visualize cell nuclei.

Following immunostaining, fluorescence was visualized using the appropriate fluorescence filter on an Olympus inverted epi-fluorescent microscope (Olympus, Melville, N.Y.). In all cases, positive staining represented fluorescence signal above control staining where the entire procedure outlined above was followed with the exception of application of a primary antibody solution. Representative images were captured using a digital color video camera and ImagePro software (Media Cybernetics, Carlsbad, Calif.). For triple-stained samples, each image was taken using only one emission filter at a time. Layered montages were then prepared using Adobe Photoshop software (Adobe, San Jose, Calif.).

TABLE 14-1
Summary of Primary Antibodies Used

| Antibody | Concentration | Vendor |
|---|---|---|
| Rat 401 (nestin) | 1:200 | Chemicon, Temecula, CA |
| TuJ1 (BIII Tubulin) | 1:500 | Sigma, St. Louis, MO |
| Tyrosine hydroxylase (TH) | 1:1000 | Chemicon |
| GABA | 1:400 | Chemicon |
| GFAP | 1:2000 | DakoCytomation, |
| | | Carpinteria, CA |
| Myelin Basic Protein (MBP) | 1:400 | Chemicon |

Quantitative Analysis of Neural Progenitor Differentiation:

Quantification of hippocampal neural progenitor differentiation was examined. A minimum of 1000 cells were counted per condition or if less, the total number of cells observed in that condition. The percentage of cells positive for a given stain was assessed by dividing the number of positive cells by the total number of cells as determined by DAPI (nuclear) staining.

Mass Spectrometry Analysis & 2D Gel Electrophoresis:

In order to identify unique, secreted factors as a result of co-culture, conditioned media samples taken prior to culture fixation were frozen down at −80° C. overnight. Samples were then applied to ultrafiltration spin devices (MW cutoff 30 kD). Retentate was applied to immunoaffinity chromatography (anti-Hu-albumin; IgY) (immunoaffinity did not remove albumin from the samples). Filtrate was analyzed by MALDI. The pass through was applied to Cibachron Blue affinity chromatography. Samples were analyzed by SDS-PAGE and 2D gel electrophoresis.

### Results

PPDC Co-Culture Stimulates Adult Neural Progenitor Differentiation:

Following culture with umbilicus- or placenta-derived cells, co-cultured neural progenitor cells derived from adult rat hippocampus exhibited significant differentiation along all three major lineages in the central nervous system. This effect was clearly observed after five days in co-culture, with numerous cells elaborating complex processes and losing their phase bright features characteristic of dividing progenitor cells. Conversely, neural progenitors grown alone in the absence of bFGF and EGF appeared unhealthy and survival was limited.

After completion of the procedure, cultures were stained for markers indicative of undifferentiated stem and progenitor cells (nestin), immature and mature neurons (TuJ1), astrocytes (GFAP), and mature oligodendrocytes (MBP). Differentiation along all three lineages was confirmed while control conditions did not exhibit significant differentiation as evidenced by retention of nestin-positive staining amongst the majority of cells. While both umbilicus- and placenta-derived cells induced cell differentiation, the degree of differentiation for all three lineages was less in co-cultures with placenta-derived cells than in co-cultures with umbilicus-derived cells.

The percentage of differentiated neural progenitors following co-culture with umbilicus-derived cells was quantified (Table 14-2). Umbilicus-derived cells significantly enhanced the number of mature oligodendrocytes (MBP) (24.0% vs. 0% in both control conditions). Furthermore, co-culture enhanced the number of GFAP+astrocytes and TuJ1+neurons in culture (47.2% and 8.7% respectively). These results were confirmed by nestin staining indicating that progenitor status was lost following co-culture (13.4% vs. 71.4% in control condition 4).

Though differentiation also appeared to be influenced by adult human fibroblasts, such cells were not able to promote the differentiation of mature oligodendrocytes nor were they able to generate an appreciable quantity of neurons. Though not quantified, fibroblasts did however, appear to enhance the survival of neural progenitors.

TABLE 14-2
Quantification of progenitor differentiation in
control vs transwell co-culture with
umbilical-derived cells (E = EGF, F = bFGF)

| | F + E/Umb | F + E/F + E | F + E/removed |
|---|---|---|---|
| Antibody | [Cond. 1] | [Cond. 4] | [Cond. 5] |
| TuJ1 | 8.7% | 2.3% | 3.6% |
| GFAP | 47.2% | 30.2% | 10.9% |
| MBP | 23.0% | 0% | 0% |
| Nestin | 13.4% | 71.4% | 39.4% |

Identification of Unique Compounds:

Conditioned media from umbilicus- and placenta-derived co-cultures, along with the appropriate controls (NPE media+I.7% serum, media from co-culture with fibroblasts), were examined for differences. Potentially unique compounds were identified and excised from their respective 2D gels.

Summary:

Co-culture of adult neural progenitor cells with umbilicus or placenta PPDCs results in differentiation of those cells. Results presented in this example indicate that the differentiation of adult neural progenitor cells following co-culture with umbilicus-derived cells is particularly profound. Specifically, a significant percentage of mature oligodendrocytes was generated in co-cultures of umbilicus-derived cells.

### Example 15

### Transplantation of Postpartum-Derived Cells

Cells derived from the postpartum umbilicus and placenta are useful for regenerative therapies. The tissue produced by postpartum-derived cells (PPDCs) transplanted into SCID mice with a biodegradable material was evaluated. The materials evaluated were Vicryl non-woven, 35/65 PCL/PGA foam, and RAD 16 self-assembling peptide hydrogel.

### Methods & Material

Cell Culture:

Placenta- and umbilicus-derived cells were grown in Growth Medium (DMEM-Iow glucose (Gibco, Carlsbad Calif.), 15% (v/v) fetal bovine serum (Cat. #SH30070.03; Hyclone, Logan, Utah), 0.001% (v/v) betamercaptoethanol (Sigma, St. Louis, Mo.), penicillin/streptomycin (Gibco)) in a gelatin-coated flasks.

Sample Preparation:

One million viable cells were seeded in 15 microliters Growth Medium onto 5 mm diameter, 2.25 mm thick Vicryl non-woven scaffolds (64.33 milligrams/cc; Lot#3547-47-1) or 5 mm diameter 35/65 PCL/PGA foam (Lot#3415-53). Cells were allowed to attach for two hours before adding more Growth Medium to cover the scaffolds. Cells were grown on scaffolds overnight. Scaffolds without cells were also incubated in medium.

RAD16 self-assembling peptides (3D Matrix, Cambridge, Mass.) was obtained as a sterile 1% (w/v) solution in water, which was mixed 1:1 with 1×10⁶ cells in 10% (w/v) sucrose (Sigma, St Louis, Mo.), 10 mM HEPES in Dulbecco's modified medium (DMEM; Gibco) immediately before use. The final concentration of cells in RAD 16 hydrogel was 1×10⁶ cells/100 microliters.

Test Material (N=4/Rx)
a. Vicryl non-woven+1×10⁶ umbilicus-derived cells
b. 35/65 PCL/PGA foam+1×10⁶ umbilicus-derived cells
c. RAD 16 self-assembling peptide+1×10⁶ umbilicus-derived cells
d. Vicryl non-woven+1×10⁶ placenta-derived cells
e. 35/65 PCL/PGA foam+1×10⁶ placenta-derived cells
f. RAD 16 self-assembling peptide+1×10⁶ placenta-derived cells
g. 35/65 PCL/PGA foam
h. Vicryl non-woven

Animal Preparation:

The animals were handled and maintained in accordance with the current requirements of the Animal Welfare Act. Compliance with the above Public Laws were accomplished by adhering to the Animal Welfare regulations (9 CFR) and conforming to the current standards promulgated in the Guide for the Care and Use of Laboratory Animals, 7th edition.

Mice (Mus Musculus)/Fox Chase SCID/Male (Harlan Sprague Dawley, Inc., Indianapolis, Ind.), 5 Weeks of Age:

All handling of the SCID mice took place under a hood. The mice were individually weighed and anesthetized with an intraperitoneal injection of a mixture of 60 milligrams/kg KETASET (ketamine hydrochloride, Aveco Co., Inc., Fort Dodge, Iowa) and 10 milligrams/kg ROMPUN (xylazine, Mobay Corp., Shawnee, Kans.) and saline. After induction of anesthesia, the entire back of the animal from the dorsal cervical area to the dorsal lumbosacral area was clipped free of hair using electric animal clippers. The area was then scrubbed with chlorhexidine diacetate, rinsed with alcohol, dried, and painted with an aqueous iodophor solution of 1% available iodine. Ophthalmic ointment was applied to the eyes to prevent drying of the tissue during the anesthetic period.

Subcutaneous Implantation Technique:

Four skin incisions, each approximately 1.0 cm in length, were made on the dorsum of the mice. Two cranial sites were located transversely over the dorsal lateral thoracic region, about 5-mm caudal to the palpated inferior edge of the scapula, with one to the left and one to the right of the vertebral column. Another two were placed transversely over the gluteal muscle area at the caudal sacro-Iumbar level, about 5-mm caudal to the palpated iliac crest, with one on either side of the midline. Implants were randomly placed in these sites in accordance with the experimental design. The skin was separated from the underlying connective tissue to make a small pocket and the implant placed (or injected for RAD16) about 1-cm caudal to the incision. The appropriate test material was implanted into the subcutaneous space. The skin incision was closed with metal clips.

Animal Housing:

Mice were individually housed in micro isolator cages throughout the course of the study within a temperature range of 64° F.-79° F. and relative humidity of 30% to 70%, and maintained on an approximate 12 hour light/12 hour dark cycle. The temperature and relative humidity were maintained within the stated ranges to the greatest extent possible. Diet consisted of Irradiated Pico Mouse Chow 5058 (Purina Co.) and water fed ad libitum.

Mice were euthanized at their designated intervals by carbon dioxide inhalation. The subcutaneous implantation sites with their overlying skin were excised and frozen for histology.

Histology:

Excised skin with implant was fixed with 10% neutral buffered formalin (Richard-Allan Kalamazoo, Mich.). Samples with overlying and adjacent tissue were centrally bisected, paraffin-processed, and embedded on cut surface using routine methods. Five-micron tissue sections were obtained by microtome and stained with hematoxylin and eosin (Poly Scientific Bay Shore, N.Y.) using routine methods.

### Results

There was minimal ingrowth of tissue into foams (without cells) implanted subcutaneously in SCID mice after 30 days. In contrast there was extensive tissue fill in foams implanted with umbilical-derived cells or placenta-derived cells. Some tissue ingrowth was observed in Vicryl non-woven scaffolds. Non-woven scaffolds seeded with umbilicus- or placenta-derived cells showed increased matrix deposition and mature blood vessels.

Summary:

Synthetic absorbable non-woven/foam discs (5.0 mm diameter×1.0 mm thick) or self-assembling peptide hydrogel were seeded with either cells derived from human umbilicus or placenta and implanted subcutaneously bilaterally in the dorsal spine region of SCID mice. The results demonstrated that postpartum-derived cells could dramatically increase good quality tissue formation in biodegradable scaffolds.

### Example 16

### Telomerase Expression in Umbilical Tissue-Derived Cells

Telomerase functions to synthesize telomere repeats that serve to protect the integrity of chromosomes and to prolong the replicative life span of cells (Liu, K, et al., PNAS, 1999; 96:5147-5152). Telomerase consists of two components, telomerase RNA template (hTER) and telomerase reverse transcriptase (hTERT). Regulation of telomerase is determined by transcription of hTERT but not hTER. Real-time polymerase chain reaction (PCR) for hTERT mRNA thus is an accepted method for determining telomerase activity of cells.

Cell Isolation.

Real-time PCR experiments were performed to determine telomerase production of human umbilical cord tissue-derived cells. Human umbilical cord tissue-derived cells were prepared in accordance the examples set forth above. Generally, umbilical cords obtained from National Disease Research Interchange (Philadelphia, Pa.) following a normal delivery were washed to remove blood and debris and mechanically dissociated. The tissue was then incubated with digestion enzymes including collagenase, dispase and hyaluronidase in culture medium at 37° C. Human umbilical cord tissue-derived cells were cultured according to the methods set forth in the examples above. Mesenchymal stem cells and normal dermal skin fibroblasts (cc-2509 lot #9F0844) were obtained from Cambrex, Walkersville, Md. A pluripotent human testicular embryonal carcinoma (teratoma) cell line nTera-2 cells (NTERA-2 c1.D1), (see, Plaia et al., Stem Cells, 2006; 24(3):531-546) was purchased from ATCC (Manassas, Va.) and was cultured according to the methods set forth above.

Total RNA Isolation.

RNA was extracted from the cells using RNeasy® kit (Qiagen, Valencia, Ca.). RNA was eluted with 50 microliters DEPC-treated water and stored at −80° C. RNA was reverse transcribed using random hexamers with the TaqMan0 reverse transcription reagents (Applied Biosystems, Foster City, Ca.) at 25° C. for 10 minutes, 37° C. for 60 minutes and 95° C. for 10 minutes. Samples were stored at −20° C.

Real-Time PCR.

PCR was performed on cDNA samples using the Applied Biosystems Assays-On-Demand™ (also known as TaqMan0 Gene Expression Assays) according to the manufacturer's specifications (Applied Biosystems). This commercial kit is widely used to assay for telomerase in human cells. Briefly, hTert (human telomerase gene) (Hs00162669) and human GAPDH (an internal control) were mixed with cDNA and TaqMan® Universal PCR master mix using a 7000 sequence detection system with ABI prism 7000 SDS software (Applied Biosystems). Thermal cycle conditions were initially 50° C. for 2 minutes and 95° C. for 10 minutes followed by 40 cycles of 95° C. for 15 seconds and 60° C. for 1 minute. PCR data was analyzed according to the manufacturer's specifications.

Human umbilical cord tissue-derived cells (ATCC Accession No. PTA-6067), fibroblasts, and mesenchymal stem cells were assayed for hTert and 18S RNA. As shown in Table 16-1, hTert, and hence telomerase, was not detected in human umbilical cord tissue-derived cells.

TABLE 16-1

| | hTert | 18S RNA |
|---|---|---|
| Umbilical cells (022803) | ND | + |
| Fibroblasts | ND | + |

ND—not detected; + signal detected

Human umbilical cord tissue-derived cells (isolate 022803, ATCC Accession No. PTA-6067) and nTera-2 cells were assayed and the results showed no expression of the telomerase in two lots of human umbilical cord tissue-derived cells while the teratoma cell line revealed high level of expression (Table 16-2).

TABLE 16-2

| | hTert | | GAPDH | | |
|---|---|---|---|---|---|
| Cell type | Exp. 1 | Exp. 2 | Exp. 1 | Exp. 2 | hTert norm |
| nTera2 | 25.85 | 27.31 | 16.41 | 16.31 | 0.61 |
| 022803 | — | — | 22.97 | 22.79 | — |

Therefore, it can be concluded that the human umbilical tissue-derived cells of the present invention do not express telomerase.

Various patents and other publications are referred to throughout the specification. Each of these publications is incorporated by reference herein, in its entirety.

Although the various aspects of the invention have been illustrated above by reference to examples and preferred embodiments, it will be appreciated that the scope of the invention is defined not by the foregoing description but by the following claims properly construed under principles of patent law.

We claim:

1. A method of administering a population of postpartum-derived cells to the eye of a subject with retinal degeneration, wherein the cell population is a homogenous population of human umbilical cord tissue-derived cells, wherein the human umbilical cord tissue-derived cells are isolated from human umbilical cord tissue substantially free of blood, wherein the population of cells secretes at least one synaptogenic factor, and wherein the synaptogenic factor is selected from TSP-1, TSP-2, and TSP-4.

2. A method of inducing synaptogenesis or neurite outgrowth in retinal neurons comprising administering a homogenouse population of human umbilical cord tissue-derived cells to the eye of a subject, wherein the cell population is isolated from human umbilical cord tissue substantially free of blood, wherein the population of human umbilical cord tissue-derived cells secretes at least one synaptogenic factor, and wherein the synaptogenic factor is selected from TSP-1, TSP-2, and TSP-4.

3. A method of developing functional synapses in retinal neurons in a subject with retinal degeneration comprising administering a composition to the eye of the subject comprising a homogeneous population of human umbilical cord tissue-derived cells, wherein the cell population is isolated from human umbilical cord tissue substantially free of blood, wherein the population of human umbilical cord tissue-derived cells secretes at least one synaptogenic factor, and wherein the synaptogenic factor is selected from TSP-1, TSP-2, and TSP-4.

4. A method of administering a population of human umbilical cord tissue-derived cells to the eye of a subject with retinal degeneration, wherein the cell population is isolated from human umbilical cord tissue substantially free of blood, wherein the population of human umbilical cord tissue-derived cells secretes at least one synaptogenic factor, and wherein the synaptogenic factor is selected from TSP-1, TSP-2, and TSP-4.

5. The method of claim 1, wherein the cell population isolated from human umbilical cord tissue substantially free of blood is capable of expansion in culture, has the potential to differentiate into cells of at least a neural phenotype, maintains a normal karyotype upon passaging, and has the following characteristics:
a) potential for 40 population doublings in culture;
b) production of CD10, CD13, CD44, CD73, and CD90;
c) lack of production of CD31, CD34, CD45, CD117, and CD141; and
d) increased expression of genes encoding interleukin 8 and reticulon 1 relative to a human cell that is a fibroblast, a mesenchymal stem cell, or an iliac crest bone marrow cell.

6. The method of claim 5, wherein the cell population is positive for HLA-A,B,C, and negative for HLA-DR,DP,DQ.

7. The method of claim 2, wherein the retinal neurons are selected from the group consisting of retinal ganglion cells, photoreceptors (rods and cones), retina amicrine cells, horizontal cells or bipolar cells.

8. The method of claim 1, wherein administration to the eye is selected from administration to the interior of an eye or administration behind the eye.

9. The method of claim 3, wherein the composition is a pharmaceutical composition.

10. The method of claim 9, wherein the pharmaceutical composition comprises a pharmaceutically acceptable carrier.

11. The method of claim 2, wherein the cell population isolated from human umbilical cord tissue substantially free of blood is capable of expansion in culture, has the potential to differentiate into cells of at least a neural phenotype, maintains a normal karyotype upon passaging, and has the following characteristics:
a) potential for 40 population doublings in culture;
b) production of CD10, CD13, CD44, CD73, and CD90;
c) lack of production of CD31, CD34, CD45, CD117, and CD141; and
d) increased expression of genes encoding interleukin 8 and reticulon 1 relative to a human cell that is a fibroblast, a mesenchymal stem cell, or an iliac crest bone marrow cell.

12. The method of claim 11, wherein the cell population is positive for HLA-A,B,C, and negative for HLA-DR,DP,DQ.

13. The method of claim 2, wherein administration to the eye is selected from administration to the interior of an eye or administration behind the eye.

14. The method of claim 3, wherein the cell population isolated from human umbilical cord tissue substantially free of blood is capable of expansion in culture, has the potential to differentiate into cells of at least a neural phenotype, maintains a normal karyotype upon passaging, and has the following characteristics:
a) potential for 40 population doublings in culture;
b) production of CD10, CD13, CD44, CD73, and CD90;
c) lack of production of CD31, CD34, CD45, CD117, and CD141; and
d) increased expression of genes encoding interleukin 8 and reticulon 1 relative to a human cell that is a fibroblast, a mesenchymal stem cell, or an iliac crest bone marrow cell.

15. The method of claim 14, wherein the cell population is positive for HLA-A,B,C, and negative for HLA-DR,DP,DQ.

16. The method of claim 3, wherein the retinal neurons are selected from the group consisting of retinal ganglion cells, photoreceptors (rods and cones), retina amicrine cells, horizontal cells or bipolar cells.

17. The method of claim 3, wherein administration to the eye is selected from administration to the interior of an eye or administration behind the eye.

18. The method of claim 4, wherein the cell population isolated from human umbilical cord tissue substantially free of blood is capable of expansion in culture, has the potential to differentiate into cells of at least a neural phenotype, maintains a normal karyotype upon passaging, and has the following characteristics:
a) potential for 40 population doublings in culture;
b) production of CD10, CD13, CD44, CD73, and CD90;
c) lack of production of CD31, CD34, CD45, CD117, and CD141; and
d) increased expression of genes encoding interleukin 8 and reticulon 1 relative to a human cell that is a fibroblast, a mesenchymal stem cell, or an iliac crest bone marrow cell.

19. The method of claim 18, wherein the cell population is positive for HLA-A,B,C, and negative for HLA-DR,DP,DQ.

20. The method of any of claim 4, wherein administration to the eye is selected from administration to the interior of an eye or administration behind the eye.
